SEQUENCE LISTING (162 sequences)

SEQ ID NO: 1 - protein, 250 aa, Artificial Sequence
  synthetic
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKPTLYNVSLVMSDTAGTCY

SEQ ID NO: 2 - protein, 269 aa, Artificial Sequence
  synthetic
MGWSCIILFLVATATGVHSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKPTLYNVSLVMSDTAGTCY

SEQ ID NO: 3 - protein, 250 aa, Artificial Sequence
  synthetic
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVCHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKPTLYNVSLVMSDTAGTCY

SEQ ID NO: 4 - protein, 269 aa, Artificial Sequence
  synthetic
MGWSCIILFLVATATGVHSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVCHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKPTLYNVSLVMSDTAGTCY

SEQ ID NO: 5 - protein, 264 aa, Artificial Sequence
  synthetic
METDTLLLWVLLLWVPGSTGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKERKCCVECPPCP

SEQ ID NO: 6 - protein, 796 aa, Artificial Sequence
  synthetic
ETVTCEDAQKTCPAVIACSSPGINGFPGKDGRDGTKGEKGEPGQGLRGLQGPPGKLGPPGNPGPSGSPGPKGQKGDPGKGGGGSGGGGSGGGGSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 7 - protein, 768 aa, Artificial Sequence
  synthetic
VEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGPPGISGPPGDPGLPGKDGDHGKPGIQGQPGPPGICDPSLCFSVIARRDPFRKGPNY

SEQ ID NO: 8 - protein, 4 aa, Artificial Sequence
  synthetic
LVLG

SEQ ID NO: 9 - protein, 18 aa, Homo sapiens
PTLYNVSLVMSDTAGTCY

SEQ ID NO: 10 - protein, 20 aa, Mus musculus
GKPTLYNVSLIMSDTGGTCY

SEQ ID NO: 11 - protein, 18 aa, Homo sapiens
PTHVNVSVVMAEVDGTCY

SEQ ID NO: 12 - protein, 269 aa, Artificial Sequence
  Synthetic
MYRMQLLSCIALSLALVTNSDKTHTCPPCPAPELLGGPSVFLFPPKPKDTLMISRTPEVTCVVVDVSHEDPEVKFNWYVDGVEVHNAKTKPREEQYNSTYRVVSVLTVCLQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLVLGPPLYNVSLVMSDTAGTCY

SEQ ID NO: 13 - protein, 4 aa, Artificial Sequence
  synthetic
CPPC

SEQ ID NO: 14 - protein, 19 aa, Homo sapiens
VPSTPPTPSPSTPPTPSPS

SEQ ID NO: 15 - protein, 6 aa, Homo sapiens
VPPPPP

SEQ ID NO: 16 - protein, 58 aa, Homo sapiens
ESPKAQASSVPTAQPQAEGSLAKATTAPATTRNTGRGGEEKKKEKEKEEQEERETKTP

SEQ ID NO: 17 - protein, 15 aa, Homo sapiens
EPKSCDKTHTCPPCP

SEQ ID NO: 18 - protein, 12 aa, Homo sapiens
ERKCCVECPPCP

SEQ ID NO: 19 - protein, 62 aa, Homo sapiens
ELKTPLGDTTHTCPRCPEPKSCDTPPPCPRCPEPKSCDTPPPCPRCPEPKSCDTPPPCPRCP

SEQ ID NO: 20 - protein, 12 aa, Homo sapiens
ESKYGPPCPSCP

SEQ ID NO: 21 - protein, 12 aa, Homo sapiens
ESKYGPPCPPCP

SEQ ID NO: 22 - protein, 4 aa, Artificial Sequence
  synthetic
CPSC

SEQ ID NO: 23 - protein, 4 aa, Artificial Sequence
  Synthetic
CPRC

SEQ ID NO: 24 - protein, 4 aa, Artificial Sequence
  Synthetic
SPPC

SEQ ID NO: 25 - protein, 4 aa, Artificial Sequence
  Synthetic
CPPS

SEQ ID NO: 26 - protein, 4 aa, Artificial Sequence
  Synthetic
SPPS

SEQ ID NO: 27 - protein, 8 aa, Artificial Sequence
  synthetic
DKTHTCAA

SEQ ID NO: 28 - protein, 11 aa, Artificial Sequence
  synthetic
DKTHTCPPCPA

SEQ ID NO: 29 - protein, 18 aa, Artificial Sequence
  synthetic
DKTHTCPPCPATCPPCPA

SEQ ID NO: 30 - protein, 25 aa, Artificial Sequence
  synthetic
DKTHTCPPCPATCPPCPATCPPCPA

SEQ ID NO: 31 - protein, 30 aa, Artificial Sequence
  synthetic
DKTHTCPPCPAGKPTLYNSLVMSDTAGTCY

SEQ ID NO: 32 - protein, 31 aa, Artificial Sequence
  synthetic
DKTHTCPPCPAGKPTHVNVSVVMAEVDGTCY

SEQ ID NO: 33 - protein, 15 aa, Artificial Sequence
  synthetic
DKTHTCCVECPPCPA

SEQ ID NO: 34 - protein, 26 aa, Artificial Sequence
  Synthetic
DKTHTCPRCPEPKSCDTPPPCPRCPA

SEQ ID NO: 35 - protein, 11 aa, Artificial Sequence
  Synthetic
DKTHTCPSCPA

SEQ ID NO: 36 - protein, 240 aa, Artificial Sequence
  Synthetic
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKPTLYNVSLVMSDTAGTCY

SEQ ID NO: 37 - protein, 240 aa, Artificial Sequence
  Synthetic
CPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKPTLYNVSLVMSDTAGTCY

SEQ ID NO: 38 - protein, 240 aa, Artificial Sequence
  Synthetic
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKPTHVNVSVVMAEVDGTCY

SEQ ID NO: 39 - protein, 240 aa, Artificial Sequence
  Synthetic
CPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKPTHVNVSVVMAEVDGTCY

SEQ ID NO: 40 - protein, 351 aa, Artificial Sequence
  Synthetic
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVALHRPDVYLLPPAREQLNLRESATITCLVTGFSPADVFVQWMQRGQPLSPEKYVTSAPMPEPQAPGRYFAHSILTVSEEEWNTGETYTCVAHEALPNRVTERTVDKSTGKPTLYNVSLVMSDTAGTCY

SEQ ID NO: 41 - protein, 351 aa, Artificial Sequence
  Synthetic
CPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKVALHRPDVYLLPPAREQLNLRESATITCLVTGFSPADVFVQWMQRGQPLSPEKYVTSAPMPEPQAPGRYFAHSILTVSEEEWNTGETYTCVAHEALPNRVTERTVDKSTGKPTLYNVSLVMSDTAGTCY

SEQ ID NO: 42 - protein, 240 aa, Artificial Sequence
  Synthetic
CPPCPAPELLGGPSVFLFPPKPKDTLMISRTPEVTCVVVDVAHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKPTLYNVSLVMSDTAGTCY

SEQ ID NO: 43 - protein, 240 aa, Artificial Sequence; identical to SEQ ID NO: 36
  Synthetic

SEQ ID NO: 44 - protein, 240 aa, Artificial Sequence; identical to SEQ ID NO: 37
  Synthetic

SEQ ID NO: 45 - protein, 240 aa, Artificial Sequence
  Synthetic
CPPCPAPEFLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKPTLYNVSLVMSDTAGTCY

SEQ ID NO: 46 - protein, 240 aa, Artificial Sequence
  Synthetic
CPPCPAPEFLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKPTLYNVSLVMSDTAGTCY

SEQ ID NO: 47 - protein, 240 aa, Artificial Sequence
  Synthetic
CPPCPAPELLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKPTLYNVSLVMSDTAGTCY

SEQ ID NO: 48 - protein, 240 aa, Artificial Sequence
  Synthetic
CPPCPAPELLGGPSVFLFPPKPKDTLMISRTPEVTCVVVDVSQEDPEVQFNWYVDGVEVHNAKTKPREEQYNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKPTLYNVSLVMSDTAGTCY

SEQ ID NO: 49 - protein, 240 aa, Artificial Sequence
  Synthetic
CPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKALPASIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKPTLYNVSLVMSDTAGTCY

SEQ ID NO: 50 - protein, 240 aa, Artificial Sequence
  Synthetic
CPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKALPSPIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKPTLYNVSLVMSDTAGTCY

SEQ ID NO: 51 - protein, 240 aa, Artificial Sequence
  Synthetic
CPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPAPIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKPTLYNVSLVMSDTAGTCY

SEQ ID NO: 52 - protein, 240 aa, Artificial Sequence
  Synthetic
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKPTLYNVSLVMSDTAGTCY

SEQ ID NO: 53 - protein, 240 aa, Artificial Sequence
  Synthetic
CPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKPTLYNVSLVMSDTAGTCY

SEQ ID NO: 54 - protein, 240 aa, Artificial Sequence
  Synthetic
CPPCPAPELLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKPTLYNVSLVMSDTAGTCY

SEQ ID NO: 55 - protein, 240 aa, Artificial Sequence
  Synthetic
CPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKALPSPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKPTLYNVSLVMSDTAGTCY

SEQ ID NO: 56 - protein, 240 aa, Artificial Sequence
  Synthetic
CPPCPAPELLGGPSVFLFPPKPKDTLMISRTPEVTCVVVDVSQEDPEVQFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKPTLYNVSLVMSDTAGTCY

SEQ ID NO: 57 - protein, 240 aa, Artificial Sequence
  Synthetic
CPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKPTLYNVSLVMSDTAGTCY

SEQ ID NO: 58 - protein, 240 aa, Artificial Sequence
  Synthetic
CPPCPAPELLGGPSVFLFPPKPKDTLMISRTPEVTCVVVDVSHEDPEVKFNWYVDGVEVHNAKTKPREEQYNSTYRVVSVLTVCHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKPTLYNVSLVMSDTAGTCY

SEQ ID NO: 59 - protein, 240 aa, Artificial Sequence
  Synthetic
CPPCPAPEFLGGPSVFLFPPKPKDTLMISRTPEVTCVVVDVSQEDPEVQFNWYVDGVEVHNAKTKPREEQFNSTYRVVSVLTVCHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKPTLYNVSLVMSDTAGTCY

SEQ ID NO: 60 - protein, 240 aa, Artificial Sequence
  Synthetic
CPPCPAPELLGGPSVFLFPPKPKDTLMISRTPEVTCVVVDVSHEDPEVKFNWYVDGVEVHNAKTKPREEQYNSTYRVVSVLTVCHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKPTHVNVSVVMAEVDGTCY

SEQ ID NO: 61 - protein, 240 aa, Artificial Sequence
  Synthetic
CPPCPAPEFLGGPSVFLFPPKPKDTLMISRTPEVTCVVVDVSQEDPEVQFNWYVDGVEVHNAKTKPREEQFNSTYRVVSVLTVCHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKPTHVNVSVVMAEVDGTCY

SEQ ID NO: 62 - protein, 351 aa, Artificial Sequence
  Synthetic
CPPCPAPELLGGPSVFLFPPKPKDTLMISRTPEVTCVVVDVSHEDPEVKFNWYVDGVEVHNAKTKPREEQYNSTYRVVSVLTVCHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVALHRPDVYLLPPAREQLNLRESATITCLVTGFSPADVFVQWMQRGQPLSPEKYVTSAPMPEPQAPGRYFAHSILTVSEEEWNTGETYTCVAHEALPNRVTERTVDKSTGKPTLYNVSLVMSDTAGTCY

SEQ ID NO: 63 - protein, 351 aa, Artificial Sequence
  Synthetic
CPPCPAPEFLGGPSVFLFPPKPKDTLMISRTPEVTCVVVDVSQEDPEVQFNWYVDGVEVHNAKTKPREEQFNSTYRVVSVLTVCHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKVALHRPDVYLLPPAREQLNLRESATITCLVTGFSPADVFVQWMQRGQPLSPEKYVTSAPMPEPQAPGRYFAHSILTVSEEEWNTGETYTCVAHEALPNRVTERTVDKSTGKPTLYNVSLVMSDTAGTCY

SEQ ID NO: 64 - protein, 240 aa, Artificial Sequence
  Synthetic
CPPCPAPELLGGPSVFLFPPKPKDTLMISRTPEVTCVVVDVAHEDPEVKFNWYVDGVEVHNAKTKPREEQYNSTYRVVSVLTVCHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKPTLYNVSLVMSDTAGTCY

SEQ ID NO: 65 - protein, 240 aa, Artificial Sequence; identical to SEQ ID NO: 36
  Synthetic

SEQ ID NO: 66 - protein, 240 aa, Artificial Sequence; identical to SEQ ID NO: 37
  Synthetic

SEQ ID NO: 67 - protein, 240 aa, Artificial Sequence; identical to SEQ ID NO: 45
  Synthetic

SEQ ID NO: 68 - protein, 240 aa, Artificial Sequence; identical to SEQ ID NO: 46
  Synthetic

SEQ ID NO: 69 - protein, 240 aa, Artificial Sequence; identical to SEQ ID NO: 47
  Synthetic

SEQ ID NO: 70 - protein, 240 aa, Artificial Sequence; identical to SEQ ID NO: 48
  Synthetic

SEQ ID NO: 71 - protein, 240 aa, Artificial Sequence; identical to SEQ ID NO: 49
  Synthetic

SEQ ID NO: 72 - protein, 240 aa, Artificial Sequence; identical to SEQ ID NO: 50
  Synthetic

SEQ ID NO: 73 - protein, 240 aa, Artificial Sequence; identical to SEQ ID NO: 51
  Synthetic

SEQ ID NO: 74 - protein, 240 aa, Artificial Sequence; identical to SEQ ID NO: 52
  Synthetic

SEQ ID NO: 75 - protein, 240 aa, Artificial Sequence; identical to SEQ ID NO: 53
  Synthetic

SEQ ID NO: 76 - protein, 240 aa, Artificial Sequence; identical to SEQ ID NO: 54
  Synthetic

SEQ ID NO: 77 - protein, 240 aa, Artificial Sequence; identical to SEQ ID NO: 55
  Synthetic

SEQ ID NO: 78 - protein, 240 aa, Artificial Sequence; identical to SEQ ID NO: 56
  Synthetic

SEQ ID NO: 79 - protein, 240 aa, Artificial Sequence; identical to SEQ ID NO: 57
  Synthetic

SEQ ID NO: 80 - protein, 240 aa, Artificial Sequence; identical to SEQ ID NO: 58
  Synthetic

SEQ ID NO: 81 - protein, 240 aa, Artificial Sequence; identical to SEQ ID NO: 59
  Synthetic

SEQ ID NO: 82 - protein, 240 aa, Artificial Sequence
  Synthetic
CPPCPAPEFLGGPSVFLFPPKPKDTLMISRTPEVTCVVVDVSHEDPEVKFNWYVDGVEVHNAKTKPREEQYNSTYRVVSVLTVCHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKPTLYNVSLVMSDTAGTCY

SEQ ID NO: 83 - protein, 240 aa, Artificial Sequence
  Synthetic
CPPCPAPEFLGGPSVFLFPPKPKDTLMISRTPEVTCVVVDVSHEDPEVKFNWYVDGVEVHNAKTKPREEQYNSTYRVVSVLTVCHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKPTLYNVSLVMSDTAGTCY

SEQ ID NO: 84 - protein, 240 aa, Artificial Sequence
  Synthetic
CPPCPAPELLGGPSVFLFPPKPKDTLMISRTPEVTCVVVDVSQEDPEVQFNWYVDGVEVHNAKTKPREEQFNSTYRVVSVLTVCHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKPTLYNVSLVMSDTAGTCY

SEQ ID NO: 85 - protein, 240 aa, Artificial Sequence
  Synthetic
CPPCPAPELLGGPSVFLFPPKPKDTLMISRTPEVTCVVVDVSQEDPEVQFNWYVDGVEVHNAKTKPREEQYNSTYRVVSVLTVCHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKPTLYNVSLVMSDTAGTCY

SEQ ID NO: 86 - protein, 240 aa, Artificial Sequence
  Synthetic
CPPCPAPEFLGGPSVFLFPPKPKDTLMISRTPEVTCVVVDVSQEDPEVQFNWYVDGVEVHNAKTKPREEQFNSTYRVVSVLTVCHQDWLNGKEYKCKVSNKALPASIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKPTLYNVSLVMSDTAGTCY

SEQ ID NO: 87 - protein, 240 aa, Artificial Sequence
  Synthetic
CPPCPAPEFLGGPSVFLFPPKPKDTLMISRTPEVTCVVVDVSQEDPEVQFNWYVDGVEVHNAKTKPREEQFNSTYRVVSVLTVCHQDWLNGKEYKCKVSNKALPSPIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKPTLYNVSLVMSDTAGTCY

SEQ ID NO: 88 - protein, 240 aa, Artificial Sequence
  Synthetic
CPPCPAPEFLGGPSVFLFPPKPKDTLMISRTPEVTCVVVDVSQEDPEVQFNWYVDGVEVHNAKTKPREEQFNSTYRVVSVLTVCHQDWLNGKEYKCKVSNKGLPAPIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKPTLYNVSLVMSDTAGTCY

SEQ ID NO: 89 - protein, 240 aa, Artificial Sequence
  Synthetic
CPPCPAPELLGGPSVFLFPPKPKDTLMISRTPEVTCVVVDVSHEDPEVKFNWYVDGVEVHNAKTKPREEQYNSTYRVVSVLTVCHQDWLNGKEYKCKVSNKALPAPIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKPTLYNVSLVMSDTAGTCY

SEQ ID NO: 90 - protein, 240 aa, Artificial Sequence
  Synthetic
CPPCPAPEFLGGPSVFLFPPKPKDTLMISRTPEVTCVVVDVSQEDPEVQFNWYVDGVEVHNAKTKPREEQFNSTYRVVSVLTVCHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKPTLYNVSLVMSDTAGTCY

SEQ ID NO: 91 - protein, 240 aa, Artificial Sequence
  Synthetic
CPPCPAPELLGGPSVFLFPPKPKDTLMISRTPEVTCVVVDVSQEDPEVQFNWYVDGVEVHNAKTKPREEQFNSTYRVVSVLTVCHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKPTLYNVSLVMSDTAGTCY

SEQ ID NO: 92 - protein, 240 aa, Artificial Sequence
  Synthetic
CPPCPAPEFLGGPSVFLFPPKPKDTLMISRTPEVTCVVVDVSQEDPEVQFNWYVDGVEVHNAKTKPREEQFNSTYRVVSVLTVCHQDWLNGKEYKCKVSNKALPSPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKPTLYNVSLVMSDTAGTCY

SEQ ID NO: 93 - protein, 240 aa, Artificial Sequence
  Synthetic
CPPCPAPELLGGPSVFLFPPKPKDTLMISRTPEVTCVVVDVSQEDPEVQFNWYVDGVEVHNAKTKPREEQYNSTYRVVSVLTVCHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKPTLYNVSLVMSDTAGTCY

SEQ ID NO: 94 - protein, 240 aa, Artificial Sequence
  Synthetic
CPPCPAPEFLGGPSVFLFPPKPKDTLMISRTPEVTCVVVDVSQEDPEVQFNWYVDGVEVHNAKTKPREEQFNSTYRVVSVLTVCHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKPTLYNVSLVMSDTAGTCY

SEQ ID NO: 95 - DNA, 750 bp, Artificial Sequence
  Synthetic
gagcccaagagctgcgacaagacccacacctgtcccccttgtcctgcccctgaactgctgggcggacctagcgtgttcctgttccccccaaagcccaaggacaccctgatgatctcccggacccccgaagtgacctgcgtggtggtggatgtgtcccacgaggaccctgaagtgaagtttaattggtacgtggacggcgtggaagtgcataacgccaagaccaagcccagagaggaacagtacaacagcacctaccgggtggtgtccgtgctgaccgtgctgcaccaggactggctgaacggcaaagagtacaagtgcaaggtgtccaacaaggccctgcctgcccccatcgagaaaaccatcagcaaggccaagggccagccccgcgaaccccaggtgtacacactgccccctagcagggacgagctgaccaagaaccaggtgtccctgacctgtctcgtgaagggcttctaccccagcgacattgccgtggaatgggagagcaacggccagcccgagaacaactacaagaccaccccccctgtgctggacagcgacggctcattcttcctgtacagcaagctgacagtggacaagagccggtggcagcagggcaacgtgttcagctgcagcgtgatgcacgaggccctgcacaaccactacacccagaagtcactgagcctgagccccggcaagcccaccctgtacaatgtgtccctcgtgatgagcgacaccgccggcacctgttac

SEQ ID NO: 96 - DNA, 750 bp, Artificial Sequence
  Synthetic
gagcccaagagctgcgacaagacccacacctgtcccccttgtcctgcccctgaactgctgggcggacctagcgtgttcctgttccccccaaagcccaaggacaccctgatgatctcccggacccccgaagtgacctgcgtggtggtggatgtgtcccacgaggaccctgaagtgaagtttaattggtacgtggacggcgtggaagtgcataacgccaagaccaagcccagagaggaacagtacaacagcacctaccgggtggtgtccgtgctgaccgtgtgccaccaggactggctgaacggcaaagagtacaagtgcaaggtgtccaacaaggccctgcctgcccccatcgagaaaaccatcagcaaggccaagggccagccccgcgaaccccaggtgtacacactgccccctagcagggacgagctgaccaagaaccaggtgtccctgacctgtctcgtgaagggcttctaccccagcgacattgccgtggaatgggagagcaacggccagcccgagaacaactacaagaccaccccccctgtgctggacagcgacggctcattcttcctgtacagcaagctgacagtggacaagagccggtggcagcagggcaacgtgttcagctgcagcgtgatgcacgaggccctgcacaaccactacacccagaagtcactgagcctgagccccggcaagcccaccctgtacaatgtgtccctcgtgatgagcgacaccgccggcacctgttac

SEQ ID NO: 97 - protein, 473 aa, Artificial Sequence
  Fc variant
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGGGGGGGGGGGGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPG

SEQ ID NO: 98 - protein, 474 aa, Artificial Sequence
  Fc variant
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGGGGGGGGGGGGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPGK

SEQ ID NO: 99 - protein, 226 aa, Artificial Sequence
  Fc variant
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVDGFYPSDIAVEWESNGQPENNYKTTPPVLDSDGSFFLVSKLTVDKSRWQQGNVFSCSVMHEALHNHYTQKSLSLSPG

SEQ ID NO: 100 - protein, 227 aa, Artificial Sequence
  Fc variant
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVDGFYPSDIAVEWESNGQPENNYKTTPPVLDSDGSFFLVSKLTVDKSRWQQGNVFSCSVMHEALHNHYTQKSLSLSPGK

SEQ ID NO: 101 - protein, 227 aa, Artificial Sequence
  Fc variant
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 102 - protein, 226 aa, Artificial Sequence
  Fc variant
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 103 - protein, 466 aa, Artificial Sequence
  Fc variant
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGSGGGSGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 104 - protein, 465 aa, Artificial Sequence
  Fc variant
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGSGGGSGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 105 - protein, 227 aa, Artificial Sequence
  Fc variant
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 106 - protein, 226 aa, Artificial Sequence
  Fc variant
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 107 - protein, 227 aa, Artificial Sequence
  Fc variant
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 108 - protein, 226 aa, Artificial Sequence
  Fc variant
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 109 - protein, 227 aa, Artificial Sequence
  Fc variant
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVEGFYPSDIAVEWESNGQPENNYKTTPPVLDSDGSFFLVSKLTVDKSRWQQGNVFSCSVMHEALHNHYTQKSLSLSPGK

SEQ ID NO: 110 - protein, 226 aa, Artificial Sequence
  Fc variant
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVEGFYPSDIAVEWESNGQPENNYKTTPPVLDSDGSFFLVSKLTVDKSRWQQGNVFSCSVMHEALHNHYTQKSLSLSPG

SEQ ID NO: 111 - protein, 227 aa, Artificial Sequence
  Fc variant
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPGK

SEQ ID NO: 112 - protein, 226 aa, Artificial Sequence
  Fc variant
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPG

SEQ ID NO: 113 - protein, 474 aa, Artificial Sequence
  Fc variant
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKSGGGSGGGSGGGSGGGSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPGK

SEQ ID NO: 114 - protein, 473 aa, Artificial Sequence
  Fc variant
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKSGGGSGGGSGGGSGGGSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPG

SEQ ID NO: 115 - protein, 474 aa, Artificial Sequence
  Fc variant
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPGKSGGGSGGGSGGGSGGGSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPGK

SEQ ID NO: 116 - protein, 473 aa, Artificial Sequence
  Fc variant
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPGKSGGGSGGGSGGGSGGGSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPG

SEQ ID NO: 117 - protein, 474 aa, Artificial Sequence
  Fc variant
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKSGGGSGGGSGGGSGGGSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 118 - protein, 473 aa, Artificial Sequence
  Fc variant
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKSGGGSGGGSGGGSGGGSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 119 - protein, 226 aa, Artificial Sequence; identical to SEQ ID NO: 99
  Fc variant

SEQ ID NO: 120 - protein, 473 aa, Artificial Sequence; identical to SEQ ID NO: 97
  Fc variant

SEQ ID NO: 121 - protein, 473 aa, Artificial Sequence
  Fc variant
DKTHTCPPCPAPELLGGPSVFLFPPKPKDTLMASRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGGGGGGGGGGGGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPG

SEQ ID NO: 122 - protein, 226 aa, Artificial Sequence
  Fc variant
DKTHTCPPCPAPELLGGPSVFLFPPKPKDTLMASRTPEVTCVVVDVSHEDPEVKFNWYVDGVEVHNAKTKPPEEQYNSTYRVVSVLTVLHQDWLNGKEYKCKVSNKALPAPIEKTISKAKGQPREPQVCTLPPSRDELTKNQVSLSCAVDGFYPSDIAVEWESNGQPENNYKTTPPVLDSDGSFFLVSKLTVDKSRWQQGNVFSCSVMHEALHNHYTQKSLSLSPG

SEQ ID NO: 123 - protein, 226 aa, Artificial Sequence
  Fc variant
DKTHTCPPCPAPELLGGPSVFLFPPKPKDTLMASRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVDGFYPSDIAVEWESNGQPENNYKTTPPVLDSDGSFFLVSKLTVDKSRWQQGNVFSCSVMHEALHNHYTQKSLSLSPG

SEQ ID NO: 124 - protein, 226 aa, Artificial Sequence
  Fc variant
DKTHTCPPCPAPELLGGPSVFLFPPKPKDTLMISRTPEVTCVVVDVSHEDPEVKFNWYVDGVEVHNAKTKPPEEQYNSTYRVVSVLTVLHQDWLNGKEYKCKVSNKALPAPIEKTISKAKGQPREPQVCTLPPSRDELTKNQVSLSCAVDGFYPSDIAVEWESNGQPENNYKTTPPVLDSDGSFFLVSKLTVDKSRWQQGNVFSCSVMHEALHNHYTQKSLSLSPG

SEQ ID NO: 125 - protein, 473 aa, Artificial Sequence
  Fc variant
DKTHTCPPCPAPELLGGPSVFLFPPKPKDTLMISRTPEVTCVVVDVSHEDPEVKFNWYVDGVEVHNAKTKPPEEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGGGGGGGGGGGGGGGDKTHTCPPCPAPELLGGPSVFLFPPKPKDTLMISRTPEVTCVVVDVSHEDPEVKFNWYVDGVEVHNAKTKPPEEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPG

SEQ ID NO: 126 - protein, 473 aa, Artificial Sequence
  Fc variant
DKTHTCPPCPAPELLGGPSVFLFPPKPKDTLMASRTPEVTCVVVDVSHEDPEVKFNWYVDGVEVHNAKTKPPEEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGGGGGGGGGGGGGGGDKTHTCPPCPAPELLGGPSVFLFPPKPKDTLMISRTPEVTCVVVDVSHEDPEVKFNWYVDGVEVHNAKTKPPEEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPG

SEQ ID NO: 127 - protein, 473 aa, Artificial Sequence
  Fc variant
DKTHTCPPCPAPELLGGPSVFLFPPKPKDTLMASRTPEVTCVVVDVSHEDPEVKFNWYVDGVEVHNAKTKPPEEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGGGGGGGGGGGGGGGDKTHTCPPCPAPELLGGPSVFLFPPKPKDTLMASRTPEVTCVVVDVSHEDPEVKFNWYVDGVEVHNAKTKPPEEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPG

SEQ ID NO: 128 - protein, 4 aa, Artificial Sequence
  Linker
GGSG

SEQ ID NO: 129 - protein, 4 aa, Artificial Sequence
  Linker
SGGG

SEQ ID NO: 130 - protein, 4 aa, Artificial Sequence
  Linker
GSGS

SEQ ID NO: 131 - protein, 4 aa, Artificial Sequence
  Linker
GGGG

SEQ ID NO: 132 - protein, 5 aa, Artificial Sequence
  Linker
GGGGS

SEQ ID NO: 133 - protein, 5 aa, Artificial Sequence
  Linker
GGGGG

SEQ ID NO: 134 - protein, 6 aa, Artificial Sequence
  Linker
GSGSGS

SEQ ID NO: 135 - protein, 6 aa, Artificial Sequence
  Linker
GGSGGS

SEQ ID NO: 136 - protein, 8 aa, Artificial Sequence
  Linker
GSGSGSGS

SEQ ID NO: 137 - protein, 8 aa, Artificial Sequence
  Linker
GGSGGGSG

SEQ ID NO: 138 - protein, 8 aa, Artificial Sequence
  Linker
GGGGGGGG

SEQ ID NO: 139 - protein, 9 aa, Artificial Sequence
  Linker
GGSGGSGGS

SEQ ID NO: 140 - protein, 10 aa, Artificial Sequence
  Linker
GSGSGSGSGS

SEQ ID NO: 141 - protein, 10 aa, Artificial Sequence
  Linker
GGGGSGGGGS

SEQ ID NO: 142 - protein, 10 aa, Artificial Sequence
  Linker
GGGGGGGGGG

SEQ ID NO: 143 - protein, 12 aa, Artificial Sequence
  Linker
GSGSGSGSGSGS

SEQ ID NO: 144 - protein, 12 aa, Artificial Sequence
  Linker
GGSGGSGGSGGS

SEQ ID NO: 145 - protein, 12 aa, Artificial Sequence
  Linker
GGSGGGSGGGSG

SEQ ID NO: 146 - protein, 12 aa, Artificial Sequence
  Linker
GGGSGGGSGGGS

SEQ ID NO: 147 - protein, 12 aa, Artificial Sequence
  Linker
GGGGGGGGGGGG

SEQ ID NO: 148 - protein, 15 aa, Artificial Sequence
  Linker
GGGGSGGGGSGGGGS

SEQ ID NO: 149 - protein, 15 aa, Artificial Sequence
  Linker
GGGGGGGGGGGGGGG

SEQ ID NO: 150 - protein, 16 aa, Artificial Sequence
  Linker
GGSGGGSGGGSGGGSG

SEQ ID NO: 151 - protein, 16 aa, Artificial Sequence
  Linker
GGGGGGGGGGGGGGGG

SEQ ID NO: 152 - protein, 18 aa, Artificial Sequence
  Linker
GGSGGGSGGGSGGGSGGS

SEQ ID NO: 153 - protein, 20 aa, Artificial Sequence
  Linker
GGSGGGSGGGSGGGSGGGSG

SEQ ID NO: 154 - protein, 20 aa, Artificial Sequence
  Linker
SGGGSGGGSGGGSGGGSGGG

SEQ ID NO: 155 - protein, 20 aa, Artificial Sequence
  Linker
GGGGGGGGGGGGGGGGGGGG

SEQ ID NO: 156 - protein, 5 aa, Artificial Sequence
  Linker
RSIAT

SEQ ID NO: 157 - protein, 8 aa, Artificial Sequence
  Linker
SACYCELS

SEQ ID NO: 158 - protein, 10 aa, Artificial Sequence
  Linker
GENLYFQSGG

SEQ ID NO: 159 - protein, 17 aa, Artificial Sequence
  Linker
AAANSSIDLISVPVDSR

SEQ ID NO: 160 - protein, 17 aa, Artificial Sequence
  Linker
RPACKIPNDLKQKVMNH

SEQ ID NO: 161 - protein, 36 aa, Artificial Sequence
  Linker
GGSAGGSGSGSSGGSSGASGTGTAGGTGSGSGTGSG

SEQ ID NO: 162 - protein, 36 aa, Artificial Sequence
  Linker
GGSGGGSEGGGSEGGGSEGGGSEGGGSEGGGSGGGS

(19) United States
(12) Patent Application Publication
(10) Pub. No.: US 2022/0332847 A1
(43) Pub. Date: Oct. 20, 2022

(54) RECOMBINANT IgG Fc MULTIMERS FOR THE TREATMENT OF IMMUNE COMPLEX-MEDIATED KIDNEY DISORDERS

(71) Applicant: CSL BEHRING LENGNAU AG, Lengnau BE (CH)

(72) Inventors: Helen Zhihui CAO, Doncaster East, Victoria (AU); Rolf SPIRIG, Bern (CH); Adrian ZUERCHER, Bern (CH); Adriana BAZ MORELLI, Carlton (AU)

(21) Appl. No.: 17/641,520

(22) PCT Filed: Sep. 11, 2020
(86) PCT No.: PCT/EP2020/075432

§ 371 Date: Mar. 9, 2022

(30) Foreign Application Priority Data
Sep. 13, 2019 (EP) 19197287.6

(51) Int. Cl. C07K 16/46; A61P 13/12

(52) U.S. Cl. CPC C07K 16/46; A61P 13/12; C07K 2319/02; C07K 2317/52; C07K 2317/60; C07K 2317/53; A61K 2039/505

(57) ABSTRACT

The invention relates to the use of recombinant IgG Fc multimers for the treatment of immune complex-mediated kidney disorders, and methods of treating immune complex-mediated kidney disorders by administering such multimers.

### FIELD OF THE INVENTION

This disclosure provides the use of recombinant IgG Fc multimers for the treatment of immune complex-mediated kidney disorders, and methods of treating immune complex-mediated kidney disorders by administering such multimers.

### BACKGROUND

Plasma-derived immunoglobulin G (IgG) is used in the clinics to treat primary and secondary immunodeficiency. In this case, IgG is administered either intravenously (IVIG) or subcutaneously (SCIG). Both are prepared from large plasma pools of more than 10,000 donors, ensuring a diverse antibody repertoire.

The administration of high doses of IVIG or SCIG (1-2 g/kg/dose) has been increasingly used for the treatment of patients with chronic or acute autoimmune and inflammatory diseases such as immune thrombocytopenia (ITP), Guillain-Barré syndrome, Kawasaki disease, chronic inflammatory demyelinating polyneuropathy (CIDP), myasthenia gravis (MG), and several other rare diseases. Additionally, off-label uses of IVIG or SCIG for several other indications are currently under exploration such as, for example, for the treatment of rheumatoid arthritis (RA).

Numerous mechanisms of action have been proposed for the anti-inflammatory effect of high-dose IVIG/SCIG. These include blockage of Fcγ receptors (FcγRs), saturation of neonatal FcR (FcRn) to enhance autoantibody clearance, up-regulation of inhibitory FcγRIIB (CD32B), scavenging of complement protein fragments and inhibition of complement fragment deposition, anti-idiotypic antibodies (Abs) in IVIG/SCIG, binding or neutralization of immune mediators (e.g. cytokines), or modulation of immune cells (e.g. induction of regulatory T cells, B cells or tolerogenic dendritic cells).

There is a need for effective and safe therapy for immune complex-mediated kidney disorders. These disorders are mediated by the inflammation of the kidney or kidney structures. They include nephritis, glomerulonephritis, interstitial nephritis, anti-glomerular basement membrane (anti-GBM) glomerulonephritis, Goodpasture's syndrome, autoimmune kidney disease, lupus nephritis, membranous nephropathy, membranoproliferative glomerulonephritis (MPGN), and Bright's disease, among others.

Anti-GBM antibodies play an essential role in the development of Goodpasture's syndrome, a life-threatening renal disease, characterized by the deposition of these antibodies along the glomerular basement membrane (Otten et al., J Immunol 2009; 183:3980-3988). These depositions lead to crescent formation, scarring and loss of real function.

Various recombinant Fc-based therapeutics are under development, including Fc fusion and multimeric proteins, which have shown efficacy in experimental animal models of arthritis, ITP and inflammatory neuropathy (Anthony et al., 2008, Science 320, 373-376; Czajkowsky et al., 2015, Sci. Rep. 5, 9526; Jain et al., 2012, Arthritis Res. 14, R192; Lin et al., 2007, J. Neuroimmunol. 186, 133-140; Niknami et al., 2013, J. Peripher. Nerv. Syst. 18, 141-152; Thiruppathi et al., 2014, J. Autoimmun. 52, 64-73).

Prospective IVIG replacement proteins comprising multiple Fc domains are described in WO2008/151088, WO2012/016073, WO2013/112986, WO2017/214321 or WO2017/019565. While envisaging a variety of different configurations of constructs with multiple Fc fragments, the main class of such constructs disclosed are so-called stradomers, which comprise Fc fragments with multimerization domains such as an IgG2 hinge region. However, no working examples are provided regarding the efficacy of the envisaged multimeric proteins in WO2008/151088.

Sun et al. describe the use of certain stradomers in the treatment of complement dependent diseases (Sun et al., J Autoimmun 2017; 84:97-108). Particularly, Sun et al. describe the testing of two recombinant Fc multimers (developed from stradomer GL-2045), which have been modified to have limited ability to interact with low/moderate affinity FcγRs, but high affinity for C1q. These compounds were found to be composed of sequential, highly ordered, Fc multimers. The authors concluded that similarly to GL-2045 these drugs functioned by serving as a C1q sink. Zuercher et al. discuss GL-2045 as being designed by fusing the human IgG2 hinge region to a human IgG1 Fc, which allows IgG1 Fc fragments to sequentially polymerize producing a heterogeneous pool of Fc multimers of various lengths or multimerization degree, essentially forming a ladder (Zuercher et al., Autoimmun Rev. 2016; 15(8):781-5).

Other Fc multimeric constructs with multimerization domains that may be useful in the invention include hexameric constructs where the IgM tailpiece is used to multimerize IgG Fc fragments. For example, WO2014/060712 discloses an Fc multimeric construct comprising an IgG1 Fc region with a truncated hinge region, a four amino acid linker at the C-terminal end of the Fc, and an IgM tailpiece, which multimerizes to predominantly hexameric structure. Mutations at Fc residues 309 and 310 (L309C and H310L) were introduced to mimic the sequence of IgM.

WO2015/132364 and WO2015/132365 disclose several Fc multimeric constructs comprising a five amino acid hinge region, an Fc region derived from IgG1, IgG4, or a hybrid of IgG1 and IgG4 CH2 and CH3 domains, and an IgM or IgA tailpiece. The disclosures are directed to improving safety and efficacy of IgG Fc multimers through the introduction of amino acid changes in the Fc regions of the fusion peptides.

Optimized hexameric Fc-μTP constructs were disclosed in WO2017/129737, which were shown to have several benefits in vivo, ex vivo, and in vitro over those described previously. Fc-μTP- and Fc-μTP-L309C-bound C1q did not induce cleavage of the complement protein C2, and therefore no C3 convertase was formed (C4b2a). Fc-μTP and Fc-μTP-L309C selectively inhibited activation of the complete classical complement pathway; no interference with the alternative pathway was observed.

Other Fc multimers were disclosed in WO2015/168643, WO2017/205436 and WO2017/205434. These Fc multimers comprise multiple Fc monomers assembled without the use of multimerization domains. Instead, two or more Fc polypeptides may be fused in a linear arrangement e.g. through a flexible peptide linker, and co-expressed with further Fc polypeptides; assembly of the Fc monomers is directed by use of selectivity modules (such as knob-in-hole or electrostatic mutations), which provide that only certain Fc polypeptides can combine.

The inventors have now surprisingly found that Fc multimers used in the present invention, including Fc-μTP-L309C, CC, SIF1, and Q1, are effective in the treatment of immune complex-mediated kidney disorders, such as for example anti-GBM glomerulonephritis.

The Fc multimers used in the present invention lack any mutation for an enhanced binding to a complement system protein, such as for example C1q, as the stradomers of Sun et al., yet they are capable of strongly inhibiting the pathogenesis of immune complex-mediated kidney disorders. For example, in a mouse model of anti-GBM glomerulonephritis, the Fc multimers of the present invention, which do not possess any mutations to induce enhanced binding to C1q, demonstrated superior efficacy in preventing albuminuria by inhibiting complement dependent cytotoxicity and antibody dependent cellular cytotoxicity.

### SUMMARY

The present disclosure provides a method of treating immune complex-mediated kidney disorders, comprising administration of Fc multimers that are assembled through the presence of multimerization domains or linkers and do not include any mutation to increase the binding affinity to a complement system protein.

In some embodiments of the present invention, the Fc multimer used in the invention comprises two to six IgG Fc fusion monomers. Each of the IgG Fc fusion monomers comprises two Fc fusion polypeptide chains and each Fc fusion polypeptide chain comprises an IgG Fc polypeptide and a multimerization domain, wherein the Fc multimer lacks any mutation to increase its binding affinity to a complement system protein. In some preferred embodiments the complement system protein is C1q. In some embodiments, the mutation that is not present in the Fc multimer used in the invention comprises at least one point mutation in an IgG1 Fc domain of the Fc multimer at any one of positions 267, 268, or 324. In some embodiments, the excluded mutation is at least one of S267E, H268F or S324T. In some embodiments, the excluded mutation comprises at least one mutation at any one of positions 267, 268, and 324, and further comprising at least one point mutation at any one of positions 233, 234, 235, 236, 238, 265, 297, 299, or 328. In some embodiments, the excluded mutation comprises at least one of N297A, T299A, P238D, E233P, G236R, L234V, E233P, L234A, L235A, P238D, D265A, D265W, N297A, N297Q, T299A, or L328F. In some embodiments, the amino acid at position 299 is not mutated from threonine to any other amino acid other than serine or cysteine. In some embodiments, the amino acid at position 298 is not mutated to any amino acid other than proline. In some embodiments, the amino acid at position 236 is not deleted. In a preferred embodiment the excluded mutations are S267E, H268F and S324T. In another preferred embodiments the excluded mutations are S267E, H268F, S324T, and N297A. In another preferred embodiment the excluded mutations are S267E, H268F, S324T, N297A, L234A, and L235A. In another preferred embodiment the excluded mutations are S267E, H268F, S324T, N297A, E233P, L234V, L235A and a deletion of G236. In another preferred embodiment the excluded mutations are S267E, H268F, S324T, L234A, L235A. In another preferred embodiment the excluded mutations are S267E, H268F, S324T, E233P, L234V, L235A, and a deletion of G236. In another preferred embodiment the excluded mutations are S267E, H268F, S324T, and D265A. In another preferred embodiment the excluded mutations are E233P, G236R, S267E, H268F, and S324T. In another preferred embodiment the excluded mutations are G236R, S267E, H268F, S324T. In another preferred embodiment the excluded mutations are E233P, G236R, S267E, H268F, S324T, and L328F. In another preferred embodiment the excluded mutations are P238D, D265G, S267E, H268F, and S324T. In another preferred embodiment the excluded mutations are P238D, D265W, S267E, H268F, and S324T. In another preferred embodiment the excluded mutations are E233P, L234V, L235A, S267E, H268F, N297A, S324T, S328F, and a deletion of G236. In another preferred embodiment the excluded mutations are S267E, H268F, S324T, and L328F. In another preferred embodiment the excluded mutations are P238D, S267E, H268F, and S324T. In another preferred embodiment the excluded mutations are P238D, S267E, H268F, S324T and N297A.

In some embodiments, the Fc multimer is not a stradomer. In some embodiments the Fc multimer does not comprise an IgG2 hinge domain as a multimerization domain.

In some embodiments, the Fc multimer comprises two to six IgG Fc fusion monomers, wherein each Fc fusion monomer comprises two Fc fusion polypeptide chains, wherein each Fc fusion polypeptide chain comprises an IgG Fc polypeptide and a multimerization domain, and wherein the multimerization domain does not include an IgG2 hinge. In some aspects of these embodiments the Fc multimer is not a stradomer.

In some embodiments of the present invention, the Fc multimer comprises two to six IgG Fc fusion monomers, wherein each Fc fusion monomer comprises two Fc fusion polypeptide chains, wherein each Fc fusion polypeptide chain comprises an IgG Fc polypeptide and a multimerization domain; and wherein the Fc multimer is not a stradomer.

In some preferred embodiments, the Fc multimer is an Fc hexamer, comprising six IgG Fc fusion monomers. In some preferred embodiments, the Fc multimer comprises an IgM tailpiece as a multimerization domain.

In some preferred embodiments, the Fc fusion polypeptide chain further comprises an IgG hinge region and the Fc fusion polypeptide chain does not comprise a Fab polypeptide.

For example, in some preferred embodiments, the Fc fusion polypeptide chain used in the invention comprises an IgG1 hinge region, an IgG1 Fc domain, and an IgM tailpiece, and does not comprise a Fab polypeptide. In a preferred embodiment, the IgM tailpiece in each Fc fusion polypeptide chain comprises 18 amino acids fused with 232 amino acids at a C-terminus of a constant region of the IgG1 Fc polypeptide. In a preferred embodiment, the Fc fusion polypeptide chain is SEQ ID NO: 1. In a further preferred embodiment, the Fc fusion polypeptide chain is SEQ ID NO:1 and has up to 5 conservative amino acid changes. In a separate preferred embodiment, the Fc fusion polypeptide chain is expressed as SEQ ID NO: 2 (corresponding to SEQ ID NO:7 of WO 2017/129737), from which the signal peptide is cleaved off during secretion and formation of the mature Fc hexamer. In some embodiments the Fc hexamer is not a stradomer.

In some preferred embodiments the mature Fc hexamer is a recombinant human Fc hexamer. In some embodiments the Fc hexamer lacks any mutation to increase its binding affinity to a complement system protein. In some preferred embodiments the complement system protein is C1q. In some embodiments, the mutation that is not present in the Fc multimer used in the present invention comprises at least one point mutation in an IgG1 Fc domain of the Fc hexamer at any one of positions 267, 268, or 324. In some embodiments, the excluded mutation is at least one of S267E, H268F or S324T. In some embodiments, the excluded mutation comprises at least one mutation at any one of positions 267, 268, and 324, and further comprising at least one point mutation at any one of positions 233, 234, 235, 236, 238, 265, 297, 299, or 328. In some embodiments, the excluded mutation comprises at least one of N297A, T299A, P238D, E233P, G236R, L234V, E233P, L234A, L235A, P238D, D265A, D265W, N297A, N297Q, T299A, or L328F. In some embodiments, the amino acid at position 299 is not mutated from threonine to any other amino acid other than serine or cysteine. In some embodiments, the amino acid at position 298 is not mutated to any amino acid other than proline. In some embodiments, the amino acid at position 236 is not deleted. In a preferred embodiment the excluded mutations are S267E, H268F and S324T. In another preferred embodiment the excluded mutations are S267E, H268F, S324T, and N297A. In another preferred embodiment the excluded mutations are S267E, H268F, S324T, N297A, L234A, and L235A. In another preferred embodiment the excluded mutations are S267E, H268F, S324T, N297A, E233P, L234V, L235A and a deletion of G236. In another preferred embodiment the excluded mutations are S267E, H268F, S324T, L234A, L235A. In another preferred embodiment the excluded mutations are S267E, H268F, S324T, E233P, L234V, L235A, and a deletion of G236. In another preferred embodiment the excluded mutations are S267E, H268F, S324T, and D265A. In another preferred embodiment the excluded mutations are E233P, G236R, S267E, H268F, and S324T. In another preferred embodiment the excluded mutations are G236R, S267E, H268F, S324T. In another preferred embodiment the excluded mutations are E233P, G236R, S267E, H268F, S324T, and L328F. In another preferred embodiment the excluded mutations are P238D, D265G, S267E, H268F, and S324T. In another preferred embodiment the excluded mutations are P238D, D265W, S267E, H268F, and S324T. In another preferred embodiment the excluded mutations are E233P, L234V, L235A, S267E, H268F, N297A, S324T, S328F, and a deletion of G236. In another preferred embodiment the excluded mutations are S267E, H268F, S324T, and L328F. In another preferred embodiment the excluded mutations are P238D, S267E, H268F, and S324T. In another preferred embodiment the excluded mutations are P238D, S267E, H268F, S324T and N297A.

In a preferred embodiment the Fc fusion polypeptide chain comprises an IgG1 hinge region, an IgG1 Fc domain, and an IgM tailpiece, wherein the IgG1 Fc domain has a cysteine instead of a leucine at position 309 (according to the EU numbering), and wherein the Fc fusion polypeptide does not comprise a Fab polypeptide and the Fc fusion polypeptide chain is SEQ ID NO:3 (corresponding to SEQ ID NO:2 of WO2017/129737). In further preferred embodiment, the Fc fusion polypeptide chain is SEQ ID NO:3 with up to 5 conservative amino acid changes. In a separate preferred embodiment, the Fc fusion polypeptide chain is expressed as SEQ ID NO:4 (corresponding to SEQ ID NO:8 of WO2017/129737), from which the signal peptide is cleaved off during secretion and formation of the mature Fc hexamer.

A further embodiment used in the present invention, is a polynucleotide encoding the Fc fusion polypeptide chain, preferably the polynucleotide also encodes a signal peptide linked to the Fc fusion polypeptide chain.

In some embodiments the Fc multimer does not contain multimerization domains.

In one embodiment the Fc multimer is comprised of four polypeptides that form three Fc monomers. The first polypeptide includes a first Fc polypeptide, a first linker, and a second Fc polypeptide. The second polypeptide includes a third Fc polypeptide, a second linker, and a fourth Fc polypeptide. The third polypeptide includes a fifth Fc polypeptide, and the fourth polypeptide includes a sixth Fc polypeptide. In this aspect, the first Fc polypeptide and the third Fc polypeptide combine to form together the first Fc monomer; the fifth Fc polypeptide and the second Fc polypeptide combine to form together the second Fc monomer; and the sixth Fc polypeptide and the fourth Fc polypeptide combine to form together the third Fc monomer.

In some embodiments, the Fc multimer does not contain an antigen-recognition region, e.g., a variable domain (e.g., V_{H}, V_{L}, a hypervariable region (HVR)) or a complementarity determining region (CDR).

In some embodiments of this aspect, each of the first and third Fc polypeptides include complementary dimerization selectivity modules that promote dimerization between the first Fc polypeptide and the third Fc polypeptide; and/or each of the second and fifth Fc polypeptides include complementary dimerization selectivity modules that promote dimerization between the second Fc polypeptide and the fifth Fc polypeptide; and/or each of the fourth and sixth Fc polypeptides include complementary dimerization selectivity modules that promote dimerization between the fourth Fc polypeptide and the sixth Fc polypeptide.

In some embodiments, the complementary dimerization selectivity modules promote selective dimerization of Fc polypeptides. In any of the Fc constructs described herein using complementary dimerization selectivity modules to promote selective dimerization of Fc polypeptides, the Fc polypeptides can have different sequences, e.g., sequences that differ by no more than 20 amino acids (e.g., no more than 15, 10 amino acids), e.g., no more than 20, 15, 10, 8, 7, 6, 5, 4, 3 or 2 amino acids, between two Fc polypeptides (i.e., between the Fc polypeptide and another Fc polypeptide of the Fc construct). For example, Fc polypeptide sequences of a construct described herein may be different because complementary dimerization selectivity modules of any of the Fc constructs can include an engineered cavity in the C_{H}3 antibody constant domain of one of the Fc polypeptides and an engineered protuberance in the C_{H}3 antibody constant domain of the other of the Fc polypeptides, wherein the engineered cavity and the engineered protuberance are positioned to form a protuberance-into-cavity pair of Fc polypeptides. In some embodiments, the Fc constructs include amino acid modifications in the C_{H}3 domain. In some embodiments, the Fc constructs include amino acid modifications in the C_{H}3 domain of the Fc polypeptides (one or more of the Fc polypeptides) for selective dimerization. Exemplary engineered cavities and protuberances are known in the art. In other embodiments, the complementary dimerization selectivity modules include an engineered (substituted) negatively-charged amino acid in the CH3 antibody constant domain of one of the Fc polypeptides and an engineered (substituted) positively-charged amino acid in the C_{H}3 antibody constant domain of the other of the Fc polypeptides, wherein the negatively-charged amino acid and the positively-charged amino acid are positioned to promote formation of an Fc domain between complementary Fc polypeptides. Exemplary complementary amino acid changes are known in the art. In some embodiments, one or more of the Fc polypeptides are the same sequence. In some embodiments, one or more of the Fc polypeptides have the same modifications. In some embodiments, only one, two, three, or four of the Fc polypeptides have the same modifications.

In some embodiments, the Fc multimer comprises four polypeptides that form three Fc monomers, wherein the first polypeptide comprises a first Fc polypeptide, a first linker, and a second Fc polypeptide, wherein the second polypeptide comprises a third Fc polypeptide, a second linker, and a fourth Fc polypeptide, wherein the third polypeptide comprises a fifth Fc polypeptide, wherein the fourth polypeptide comprises a sixth Fc polypeptide, wherein the first Fc polypeptide and the third Fc polypeptide form the first Fc monomer, wherein the fifth Fc polypeptide and the second Fc polypeptide form the second Fc monomer, and wherein the sixth Fc polypeptide and fourth Fc polypeptide form the third Fc monomer.

In some embodiments, the Fc multimer includes at least two Fc monomers joined through a linker. In some embodiments, the Fc multimer includes at least one linker. A linker can be an amino acid spacer including 3-200 amino acids (e.g., 3-150, 3-100, 3-60, 3-50, 3-40, 3-30, 3-20, 3-10, 3-8, 3-5, 4-30, 5-30, 6-30, 8-30, 10-20, 10-30, 12-30, 14-30, 20-30, 15-25, 15-30, 18-22 and 20-30 amino acids). Suitable peptide linkers are known in the art, and include, for example, peptide linkers containing flexible amino acid residues such as glycine and serine. In certain embodiments, a linker can contain motifs, e.g., single, multiple or repeating motifs, of GS, GGS, GGSG, GGGGS, GGG, GGGG. In certain embodiments, a linker can include GS, GGS, GGSG, GGGGS, GGG, GGGG or any of SEQ ID NOs: 128-155. In some embodiments, a linker is used to connect two Fc polypeptides in a tandem series. In other embodiments, a linker is used to connect CL and C_{H}, antibody constant domains. In other embodiments, a linker can also contain amino acids other than glycine and serine, e.g., SEQ ID NOs: 156-162.

In some embodiments described herein, the Fc multimer can comprise a polypeptide comprising SEQ ID NO: 97-127 or SEQ ID Nos: 97-127 with up to 10 (e.g., up to 9, 8, 7, 6, 5, 4, 3, 2, or 1) single amino acid modifications (e.g., substitutions, e.g., conservative substitutions). In some embodiments, the Fc polypeptides of an Fc domain of a construct can have different sequences, e.g., sequences that differ by no more than 20 amino acids (e.g., no more than 15, 10 amino acids), e.g., no more than 20, 15, 10, 8, 7, 6, 5, 4, 3 or 2 amino acids, between two Fc polypeptides (i.e., between the Fc polypeptide and another Fc polypeptide of the Fc construct).

In some embodiments, one or more polypeptides in an Fc construct contain a terminal lysine residue. In some embodiments, one or more Fc polypeptides in an Fc construct do not contain a terminal lysine residue. In some embodiments, all of the Fc polypeptides in an Fc construct contain a terminal lysine residue. In some embodiments, all of the Fc polypeptides in an Fc construct do not contain a terminal lysine residue. In some embodiments, the terminal lysine residue in an Fc polypeptide which comprises, consists of, or consists essentially of the sequence of any one of SEQ ID NOs: 98, 100, 101, 103, 105, 107, 109, 111, 113, 115, and 117 may be removed to generate a corresponding Fc polypeptide that does not contain a terminal lysine residue. In some embodiments, a terminal lysine residue may be added to an Fc polypeptide comprising, consisting of, or consisting essentially of the sequence of SEQ ID NO: 97, 99, 102, 104, 106, 108, 110, 112, 114, 116, and 118-127 to generate a corresponding Fc polypeptide that contains a terminal lysine residue.

In some embodiments, the Fc multimer lacks any mutation to increase binding affinity of the Fc multimer to a complement system protein. In some preferred embodiments, the complement system protein is C1q. In some embodiments the mutation that is not present in the Fc multimer used in the present invention comprises at least one point mutation in an IgG1 Fc domain of the Fc multimer at any one of positions 267, 268, or 324. In some embodiments, the excluded mutation is at least one of S267E, H268F or S324T. In some embodiments, the excluded mutation comprises at least one mutation at any one of positions 267, 268, and 324, and further comprising at least one point mutation at any one of positions 233, 234, 235, 236, 238, 265, 297, 299, or 328. In some embodiments, the excluded mutation comprises at least one of N297A, T299A, P238D, E233P, G236R, L234V, E233P, L234A, L235A, P238D, D265A, D265W, N297A, N297Q, T299A, or L328F. In some embodiments, the amino acid at position 299 is not mutated from threonine to any other amino acid other than serine or cysteine. In some embodiments, the amino acid at position 298 is not mutated to any amino acid other than proline. In some embodiments, the amino acid at position 236 is not deleted. In a preferred embodiment the excluded mutations are S267E, H268F and S324T. In another preferred embodiments the excluded mutations are S267E, H268F, S324T, and N297A. In another preferred embodiment the excluded mutations are S267E, H268F, S324T, N297A, L234A, and L235A. In another preferred embodiment the excluded mutations are S267E, H268F, S324T, N297A, E233P, L234V, L235A and a deletion of G236. In another preferred embodiment the excluded mutations are S267E, H268F, S324T, L234A, L235A. In another preferred embodiment the excluded mutations are S267E, H268F, S324T, E233P, L234V, L235A, and a deletion of G236. In another preferred embodiment the excluded mutations are S267E, H268F, S324T, and D265A. In another preferred embodiment the excluded mutations are E233P, G236R, S267E, H268F, and S324T. In another preferred embodiment the excluded mutations are G236R, S267E, H268F, S324T. In another preferred embodiment the excluded mutations are E233P, G236R, S267E, H268F, S324T, and L328F. In another preferred embodiment the excluded mutations are P238D, D265G, S267E, H268F, and S324T. In another preferred embodiment the excluded mutations are P238D, D265W, S267E, H268F, and S324T. In another preferred embodiment the excluded mutations are E233P, L234V, L235A, S267E, H268F, N297A, S324T, S328F, and a deletion of G236. In another preferred embodiment the excluded mutations are S267E, H268F, S324T, and L328F. In another preferred embodiment the excluded mutations are P238D, S267E, H268F, and S324T. In another preferred embodiment the excluded mutations are P238D, S267E, H268F, S324T and N297A.

In a preferred embodiment, the Fc multimer is administered intravenously or non-intravenously. In one embodiment, the Fc multimer is administered subcutaneously. In one embodiment, the Fc multimer is applied orally, or intrathecally, or intrapulmonarily by nebulization.

In a preferred embodiment, the Fc multimer is administered in an amount ranging from about 3 mg/kg to about 200 mg/kg. In one embodiment, the Fc multimer is administered in an amount ranging from about 1 mg/kg to about 500 mg/kg. All doses are per kg of bodyweight of the subject to which the Fc multimer is administered.

In an alternative embodiment, the Fc multimer used in the invention is a stradomer where IgG Fc fragments are provided with a multimerization domain, preferably an IgG2 hinge region, as disclosed in WO2008/151088, WO2012/016073, WO2017/214321 or WO2017/019565, but wherein the Fc multimer lacks any mutation to increase its binding affinity to a complement system protein. In some preferred embodiments, the complement system protein is C1q. In a preferred embodiment, the Fc multimer is produced by expressing polypeptide chains comprising SEQ ID NO:5, whereby the mature Fc multimer comprises residues 21 to 264 of SEQ ID NO:5.

It is to be understood that the foregoing general description and the following detailed description are exemplary and explanatory only, and are intended to provide further, non-limiting explanation of the disclosure.

### BRIEF DESCRIPTION OF DRAWING(S)

FIG. 1 shows the effect of the Fc-μTP-L309C hexamer (CSL777), generated in CHO cells, in an in vivo model of anti-GBM glomerulonephritis, as represented by the levels of albumin detected in mice urine by an ELISA kit.

FIG. 2 shows the effect of the Fc-μTP-L309C hexamer (CSL777), generated in CHO cells (Fc-μTP-L309C (CHO)) and HEK293 cells (Fc-μTP-L309C (HEK)), and a mutant hexamer with reduced C1q binding capacity (K322A) in an in vivo model of anti-GBM glomerulonephritis, as represented by the levels of albumin detected in mice urine by an ELISA kit.

FIG. 3 shows the effect of the Fc-μTP-L309C hexamer (CSL777) and other Fc multimers (CC, SIF1, and Q1) in an in vivo model of anti-GBM glomerulonephritis, as represented by the levels of albumin detected in mice urine by an ELISA kit.

FIG. 4 depicts a dose-response effect of the Fc-μTP-L309C hexamer (CSL777) in an in vivo model of anti-GBM glomerulonephritis, as represented by the levels of albumin detected in mice urine by an ELISA kit.

FIG. 5: Sequences from WO2017/129737

FIG. 6: Other hexamer sequences used in embodiments of the invention

FIG. 7: Stradomer sequences used in embodiments of the invention

FIG. 8: Recombinant Fc compounds as disclosed in WO2017/172853, used in embodiments of the present invention

FIG. 9: Examples of suitable hinge regions used in Fc multimers used in embodiments of the invention

FIG. 10: Sequences for trivalent Fc multimers used in embodiments of the invention

### DETAILED DESCRIPTION

The following detailed description and examples illustrate certain embodiments of the present disclosure. Those of skill in the art will recognize that there are numerous variations and modifications of this disclosure that are encompassed by its scope. Accordingly, the description of certain embodiments should not be deemed as limiting.

In some embodiments, an Fc multimer comprises two to six IgG Fc fusion monomers, wherein each Fc fusion monomer comprises two Fc fusion polypeptide chains, and each Fc fusion polypeptide chain comprises an IgG Fc polypeptide and a multimerization domain; and wherein the Fc multimer lacks any mutation to increase binding affinity of the Fc multimer to a complement system protein.

In some embodiments, a mutation which increases binding affinity of the Fc multimer to a complement system protein and is therefore excluded from the Fc multimers used in the present invention may be at least one point mutation in one or more IgG1 Fc domains of the Fc multimer. In some embodiments the excluded mutation corresponds to a point mutation at positions 267, 268, and/or 324 of the IgG1 Fc domain. In some embodiments, the excluded mutation is at least one of S267E, H268F and S324T. In some embodiments, the excluded mutation may be one or more point mutations corresponding to at least one of positions 267, 268, and/or 324 and further comprising at least one point mutation at position 233, and/or position 234, and/or position 235, and/or position 236, and/or position 238, and/or position 265, and/or position 297, and/or position 299, and/or position 328. In some embodiments, the excluded mutation may be at least one point mutation at position 233, and/or position 234, and/or position 235, and/or position 236, and/or position 238, and/or position 265, and/or position 297, and/or position 299, and/or position 328. In some embodiments, the excluded mutation is at least one of N297A, T299A, P238D, E233P, G236R, L234V, E233P, L234A, L235A, P238D, D265A, D265W, N297A, N297Q, T299A, and L328F. In some embodiments, the amino acid at position 299 is not mutated from threonine to any other amino acid other than serine or cysteine. In some embodiments, the amino acid at position 298 is not mutated to any amino acid other than proline. In some embodiments, the amino acid at position 236 is not deleted. In some embodiments the complement system protein may be C1q.

In some embodiments, the Fc multimer is not a stradomer. In some embodiments the Fc multimer does not include an IgG2 hinge as a multimerization domain.

In some embodiments the Fc multimer comprises two to six IgG Fc fusion monomers, wherein each Fc fusion monomer comprises two Fc fusion polypeptide chains, and each Fc fusion polypeptide chain comprises an IgG Fc polypeptide and a multimerization domain, and wherein the multimerization domain does not include an IgG2 hinge. In a further embodiment the Fc multimer is not a stradomer.

In some embodiments the Fc multimer comprises two to six IgG Fc fusion monomers, wherein each Fc fusion monomer comprises two Fc fusion polypeptide chains, and each Fc fusion polypeptide chain comprises an IgG Fc polypeptide and a multimerization domain; and wherein the Fc multimer is not a stradomer.

In some embodiments, the Fc multimer comprises six IgG fusion monomers. In some embodiments the Fc multimer comprises an IgGM tailpiece as a multimerization domain.

In some embodiments the Fc multimer is a recombinant human Fc hexamer. In some embodiments the recombinant human Fc hexamer comprises six human IgG1 Fc fusion monomers, wherein each Fc fusion monomer comprises two human Fc fusion polypeptide chains and each Fc fusion polypeptide chain comprises a human IgG1 Fc polypeptide and a human IgM tailpiece, and further wherein the IgM tailpiece in each Fc fusion polypeptide chain comprises 18 amino acids fused with 232 amino acids at a C-terminus of a constant region of the IgG1 Fc polypeptide.

In some embodiments the recombinant human Fc hexamer lacks any mutation to increase its binding affinity to a complement system protein. In some preferred embodiments the complement system protein is C1q. In some embodiments, the mutation that is not present in the Fc multimers used in the present invention comprises at least one point mutation in an IgG1 Fc domain of the Fc hexamer at any one of positions 267, 268, or 324. In some embodiments, the excluded mutation is at least one of S267E, H268F or S324T. In some embodiments, the excluded mutation comprises at least one mutation at any one of positions 267, 268, and 324, and further comprising at least one point mutation at any one of positions 233, 234, 235, 236, 238, 265, 297, 299, or 328. In some embodiments, the excluded mutation comprises at least one of N297A, T299A, P238D, E233P, G236R, L234V, E233P, L234A, L235A, P238D, D265A, D265W, N297A, N297Q, T299A, or L328F. In some embodiments, the amino acid at position 299 is not mutated from threonine to any other amino acid other than serine or cysteine. In some embodiments, the amino acid at position 298 is not mutated to any amino acid other than proline. In some embodiments, the amino acid at position 236 is not deleted. In a preferred embodiment the excluded mutations are S267E, H268F and S324T. In another preferred embodiment the excluded mutations are S267E, H268F, S324T, and N297A. In another preferred embodiment the excluded mutations are S267E, H268F, S324T, N297A, L234A, and L235A. In another preferred embodiment the excluded mutations are S267E, H268F, S324T, N297A, E233P, L234V, L235A and a deletion of G236. In another preferred embodiment the excluded mutations are S267E, H268F, S324T, L234A, L235A. In another preferred embodiment the excluded mutations are S267E, H268F, S324T, E233P, L234V, L235A, and a deletion of G236. In another preferred embodiment the excluded mutations are S267E, H268F, S324T, and D265A. In another preferred embodiment the excluded mutations are E233P, G236R, S267E, H268F, and S324T. In another preferred embodiment the excluded mutations are G236R, S267E, H268F, S324T. In another preferred embodiment the excluded mutations are E233P, G236R, S267E, H268F, S324T, and L328F. In another preferred embodiment the excluded mutations are P238D, D265G, S267E, H268F, and S324T. In another preferred embodiment the excluded mutations are P238D, D265W, S267E, H268F, and S324T. In another preferred embodiment the excluded mutations are E233P, L234V, L235A, S267E, H268F, N297A, S324T, S328F, and a deletion of G236. In another preferred embodiment the excluded mutations are S267E, H268F, S324T, and L328F. In another preferred embodiment the excluded mutations are P238D, S267E, H268F, and S324T. In another preferred embodiment the excluded mutations are P238D, S267E, H268F, S324T and N297A. In some embodiments the recombinant human Fc hexamer is not a stradomer.

In some embodiments the Fc multimer comprises four polypeptides that form three Fc monomers, wherein the first polypeptide comprises a first Fc polypeptide, a first linker, and a second Fc polypeptide, wherein the second polypeptide comprises a third Fc polypeptide, a second linker, and a fourth Fc polypeptide, wherein the third polypeptide comprises a fifth Fc polypeptide, wherein the fourth polypeptide comprises a sixth Fc polypeptide, wherein the first Fc polypeptide and the third Fc polypeptide form the first Fc monomer, wherein the fifth Fc polypeptide and the second Fc polypeptide form the second Fc monomer, and wherein the sixth Fc polypeptide and fourth Fc polypeptide form the third Fc monomer.

In a further embodiment the Fc multimer lacks any mutation to increase its binding to a complement system protein. In some preferred embodiments the complement system protein is C1q. In some embodiments, the mutation not present in the Fc multimers used in the present invention comprises at least one point mutation in an IgG1 Fc domain of the Fc multimer at any one of positions 267, 268, or 324. In some embodiments, the excluded mutation is at least one of S267E, H268F or S324T. In some embodiments, the excluded mutation comprises at least one mutation at any one of positions 267, 268, and 324, and further comprising at least one point mutation at any one of positions 233, 234, 235, 236, 238, 265, 297, 299, or 328. In some embodiments, the excluded mutation comprises at least one of N297A, T299A, P238D, E233P, G236R, L234V, E233P, L234A, L235A, P238D, D265A, D265W, N297A, N297Q, T299A, or L328F. In some embodiments, the amino acid at position 299 is not mutated from threonine to any other amino acid other than serine or cysteine. In some embodiments, the amino acid at position 298 is not mutated to any amino acid other than proline. In some embodiments, the amino acid at position 236 is not deleted. In a preferred embodiment the excluded mutations are S267E, H268F and S324T. In another preferred embodiments the excluded mutations are S267E, H268F, S324T, and N297A. In another preferred embodiment the excluded mutations are S267E, H268F, S324T, N297A, L234A, and L235A. In another preferred embodiment the excluded mutations are S267E, H268F, S324T, N297A, E233P, L234V, L235A and a deletion of G236. In another preferred embodiment the excluded mutations are S267E, H268F, S324T, L234A, L235A. In another preferred embodiment the excluded mutations are S267E, H268F, S324T, E233P, L234V, L235A, and a deletion of G236. In another preferred embodiment the excluded mutations are S267E, H268F, S324T, and D265A. In another preferred embodiment the excluded mutations are E233P, G236R, S267E, H268F, and S324T. In another preferred embodiment the excluded mutations are G236R, S267E, H268F, S324T. In another preferred embodiment the excluded mutations are E233P, G236R, S267E, H268F, S324T, and L328F. In another preferred embodiment the excluded mutations are P238D, D265G, S267E, H268F, and S324T. In another preferred embodiment the excluded mutations are P238D, D265W, S267E, H268F, and S324T. In another preferred embodiment the excluded mutations are E233P, L234V, L235A, S267E, H268F, N297A, S324T, S328F, and a deletion of G236. In another preferred embodiment the excluded mutations are S267E, H268F, S324T, and L328F. In another preferred embodiment the excluded mutations are P238D, S267E, H268F, and S324T. In another preferred embodiment the excluded mutations are P238D, S267E, H268F, S324T and N297A.

The term “Fc monomer,” as used herein, is defined as a portion of an immunoglobulin G (IgG) heavy chain constant region containing the heavy chain CH2 and CH3 domains of IgG, or a variant or fragment thereof. The IgG CH2 and CH3 domains are also referred to as Cγ2 and Cγ3 domains respectively.

The Fc monomer may be comprised of two identical Fc polypeptides linked by disulfide bonds between cysteine residues in the N-terminal parts of the polypeptides. The arrangement of the disulfide linkages described for IgG pertain to natural human antibodies. There may be some variation among antibodies from other vertebrate species, although such antibodies may be suitable in the context of the present invention. The Fc polypeptides may be produced by recombinant expression techniques and associate by disulfide bonds as occurs in native antibodies. Alternatively, one or more new cysteine residues may be introduced in an appropriate position in the Fc polypeptide to enable disulfide bonds to form.

In one embodiment, the Fc monomer used in the present invention comprises two identical polypeptide chains comprising the human IgG1 CH2 and CH3 domains as described in WO2017/129737.

In another embodiment, the Fc monomer used in the present invention includes the entire CH2 and CH3 domains and is truncated at the N-terminus end of CH2 or the C-terminus end of CH3, respectively as disclosed in WO2017/129737. Typically, the Fc monomer lacks the Fab polypeptide of the immunoglobulin. The Fab polypeptide is comprised of the CH1 domain and the heavy chain variable region domain.

The Fc monomer used in the present invention may comprise more than the CH2 and CH3 portion of an immunoglobulin. For example, in one embodiment, the monomer includes the hinge region of the immunoglobulin, a fragment or variant thereof, or a modified hinge region. A native hinge region is the region of the immunoglobulin which occurs between CH1 and CH2 domains in a native immunoglobulin. A variant or modified hinge region is any hinge that differs in length and/or composition from the native hinge region. Such hinges can include hinge regions from other species. Other modified hinge regions comprise a complete hinge region derived from an antibody of a different class or subclass from that of the Fc portion. Alternatively, the modified hinge region comprises part of a natural hinge or a repeating unit in which each unit in the repeat is derived from a natural hinge region. In another alternative, the natural hinge region is altered by increasing or decreasing the number of cysteine residues. Other modified hinge regions are entirely non-natural and are designed to possess desired properties such as length, cysteine composition, and flexibility.

A number of modified hinge regions have been described for use in the present invention, for example in U.S. Pat. No. 5,677,425, WO1998/25971, WO1999/15549, WO2005/003169, WO2005/003170 and WO2005/003171.

The Fc polypeptide in the Fc multimer used in one embodiment of the present invention possesses a human IgG1 hinge region at its N-terminus. In one embodiment, the hinge region has the sequence of residues 1 to 15 of SEQ ID NO:1.

The Fc polypeptide chain used in some embodiments of the present invention is expressed comprising a signal peptide as disclosed in WO 2017/129737. The signal peptide directs the secretion of the Fc polypeptide chain and thereafter is cleaved from the remainder of the Fc polypeptide chain.

The Fc polypeptide used in an embodiment of the present invention includes a signal peptide fused to the N-terminus of the hinge region. The signal peptide may have the sequence of residues 1 to 19 of SEQ ID NO:2; however, the skilled person will be aware that other signal sequences that direct secretion of proteins from mammalian cells may also be used.

In order to improve formation of multimeric structures of two or more Fc monomers, the Fc polypeptide is fused to a tailpiece, which causes the monomer units to assemble into a multimer. The product of the fusion of the Fc polypeptide to the tailpiece is the “Fc fusion polypeptide,” as used herein. As Fc polypeptides dimerize to form Fc monomers, Fc fusion polypeptides likewise dimerize to form Fc fusion monomers.

A “Fc fusion monomer” as used herein therefore comprises two Fc fusion polypeptide chains and each Fc fusion polypeptide chain comprises an IgG Fc polypeptide and an IgM tailpiece or an IgA tailpiece, preferably an IgM tailpiece.

Suitable tailpieces are derived from IgM or IgA. IgM and IgA occur naturally in humans as covalent multimers of the common H₂L₂ antibody unit. IgM occurs as a pentamer when it has incorporated a J-chain, or as a hexamer when it lacks a J-chain. IgA occurs as monomers and forms dimers. The heavy chains of IgM and IgA each possess a respective 18 amino acid extension to the C-terminal constant domain, known as a tailpiece. This tailpiece includes a cysteine residue that forms a disulfide bond between heavy chains in the polymer, and is believed to have an important role in polymerization. The tailpiece also contains a glycosylation site.

The tailpiece of the present disclosure comprises any suitable amino acid sequence. The tailpiece is a tailpiece found in a naturally occurring antibody, or alternatively, it is a modified tailpiece which differs in length and/or composition from a natural tailpiece. Other modified tailpieces are entirely non-natural and are designed to possess desired properties for multimerization, such as length, flexibility, and cysteine composition.

The tailpiece in the Fc multimer used in an embodiment of the present invention comprises all or part of the 18 amino acid sequence from human IgM as shown in residues 233 to 250 of SEQ ID NO:1 and in SEQ ID NO:9. Alternatively, the tailpiece may be a fragment or variant of the human IgM tailpiece.

The tailpiece in the Fc multimer used in one embodiment of the present invention is fused directly to the C-terminus of a constant region of the Fc polypeptide to form the Fc fusion polypeptide. Alternatively, the tailpiece is fused to a 232 amino acid segment at the C-terminus of the constant region of the Fc polypeptide, preferably the human IgG1 Fc polypeptide. Alternatively, the tailpiece is fused indirectly by means of an intervening amino acid sequence. For example, a short linker sequence may be provided between the tailpiece and the Fc polypeptide. A linker sequence may be between 1 and 20 amino acids in length.

Formation of multimeric structures may be further improved by mutating leucine 309 of the Fc portion of the Fc fusion polypeptide to cysteine. The L309C mutation allows for additional disulfide bond formation between the Fc fusion monomers, which further promotes multimerization of the Fc fusion monomers. The residues of the IgG Fc portion are numbered according to the EU numbering system for IgG, described in Edelman G M et al (1969), Proc Natl Acad Sci 63, 78-85; see also Kabat et al., 1983, Sequences of proteins of immunological interest, US Department of Health and Human Services, National Institutes of Health, Washington, D.C. Leu 309 of IgG corresponds by sequence homology to Cys 414 in Cμ3 domain of IgM and Cys 309 in the Cα2 domain of IgA.

Other mutations additionally, or alternatively, are introduced in the Fc fusion polypeptide to achieve desirable effects. The term “mutation,” as used herein, includes a substitution, addition, or deletion of one or more amino acids. In some embodiments, as described in WO2017/129737, the Fc fusion polypeptide comprises up to 20, up to 10, up to 5, or up to 2 amino acid mutations.

The mutations in the Fc multimer used in one embodiment of the present invention are conservative amino acid changes as described in WO2017/129737. The term “conservative amino acid changes,” as used herein, refers to the change of an amino acid to a different amino acid with similar biochemical properties, such as charge, hydrophobicity, structure, and/or size. The Fc fusion polypeptide used in an embodiment of the present invention comprises up to 20, up to 10, up to 5, or up to 2 conservative amino acid changes. For example, the Fc fusion polypeptide comprises up to 5 conservative amino acid changes.

A conservative amino acid change includes a change amongst the following groups of residues: Val, Ile, Leu, Ala, Met; Asp, Glu; Asn, Gln; Ser, Thr, Gly, Ala; Lys, Arg, His; and Phe, Tyr, Trp.

A “variant,” when used herein to describe a peptide, protein, or fragment thereof, may have modified amino acids. Suitable modifications include acetylation, glycosylation, hydroxylation, methylation, nucleotidylation, phosphorylation, ADP-ribosylation, and other modifications known in the art. Such modifications may occur post-translationally where the peptide is made by recombinant techniques. Otherwise, modifications may be made to synthetic peptides using techniques known in the art. Modifications may be included prior to incorporation of an amino acid into a peptide. Carboxylic acid groups may be esterified or may be converted to an amide, an amino group may be alkylated, for example methylated. A variant may also be modified post-translationally, for example to remove or add carbohydrate side-chains or individual sugar moieties.

The term “Fc multimer,” as used herein, describes two or more polymerized Fc monomers. The Fc monomers may be Fc fusion monomers. An Fc multimer comprises two to six Fc monomers, producing Fc dimers, Fc trimers, Fc tetramers, Fc pentamers, and Fc hexamers. Fc monomers associate into polymers having different numbers of monomer units.

As used herein, the term “joined” is used to describe the combination or attachment of two or more elements, components, or protein domains, e.g., polypeptides, by means including chemical conjugation, recombinant means, and chemical bonds, e.g., disulfide bonds and amide bonds. For example, two single polypeptides can be joined to form one contiguous protein structure through chemical conjugation, a chemical bond, a peptide linker, or any other means of covalent linkage. In some embodiments, a first Fc polypeptide is joined to a second Fc polypeptide by way of a linker, e.g., a peptide linker, wherein the N-terminus of the peptide linker is joined to the C-terminus of the first Fc polypeptide through a chemical bond, e.g., a peptide bond, and the C-terminus of the peptide linker is joined to the N-terminus of the second Fc polypeptide through a chemical bond, e.g., a peptide bond.

As used herein, the term “linker” refers to a “spacer” between two elements, e.g., Fc polypeptides. The term “spacer” refers to a moiety (e.g., a polyethylene glycol (PEG) polymer) or an amino acid sequence (e.g., a 3-200 amino acid, 3-150 amino acid, or 3-100 amino acid sequence) occurring between two polypeptides or polypeptide domains to provide space and/or flexibility between the two polypeptides or polypeptide domains. An amino acid spacer is part of the primary sequence of a polypeptide (e.g., joined to the spaced polypeptides or polypeptide domains via the polypeptide backbone). A “linker” can occur between two Fc polypeptides to provide flexibility and/or space.

In WO2017/129737, the majority of Fc multimer is an Fc hexamer. As used herein, the term “majority” refers to greater than 50%, greater than 60%, greater than 70%, greater than 80%, or greater than 90%. In one embodiment, greater than 80% of the Fc multimer is an Fc hexamer.

If Fc multimers containing a specific number of monomers are required, Fc multimers can be separated according to molecular size, for example by gel filtration (size exclusion chromatography).

In one embodiment, the Fc multimers used in the present invention are the prospective IVIG replacement proteins comprising multiple Fc domains, as described, for example, in WO2008/151088, or WO2012/016073.

In another embodiment, as described in WO2008/151088, the multimeric Fc is a stradomer with a multimerization domain, such as an IgG2 hinge region, wherein the stradomer lacks any mutation to increase its binding affinity to a complement system protein. In a preferred embodiment the complement system protein is C1q.

In one embodiment, the Fc multimer used in the invention is a compound comprising two or more multimerized units, wherein each of said units comprises a multimerizing region and a region comprising at least one Fc domain that is capable of binding to a Fcγ receptor, wherein each of said units comprises a multimerizing region monomer and a region comprising at least one Fc polypeptide, wherein the dimerization of the two monomers forms a multimerizing region and a region comprising at least one Fc domain that is capable of binding to a Fcγ receptor, wherein the multimerizing regions of the two or more units multimerize to form the compound, and wherein the compound is capable of binding to a first Fcγ receptor through a first Fc domain and to a second Fcγ receptor through a second Fc domain, wherein the multimerizing region is selected from the group consisting of an IgG2 hinge, an IgE CH2 domain, a leucine zipper, an isoleucine zipper and a zinc finger, and wherein each of the regions comprising at least one Fc domain that is capable of binding to a Fcγ receptor comprises an IgG1 hinge, an IgG1 CH2 domain and an IgG1 CH3 domain, as disclosed, for example, in WO2008/151088, WO2012/016073 and WO2017/214321, hereby incorporated in their entirety by reference. However, the embodiments of the present invention lack any mutation to increase their binding affinity to a complement system protein such as for example C1q. In some embodiments the multimerizing region is an IgG2 hinge region, for example the IgG2 12 amino acid hinge region ERKCCVECPPCP (residues 253 to 264 in SEQ ID NO: 5). More preferably, the Fc multimer is obtained by expression of a polypeptide of SEQ ID NO: 5 (SEQ ID NO: 4 in WO 2012/016073), which multimerizes spontaneously through the IgG2 hinge multimerization domain.

In another alternative embodiment, the recombinant Fc compound used in the present invention is as disclosed in WO2017/172853, hereby incorporated in its entirety by reference. Preferably, the recombinant Fc compound comprises a single chain Fc peptide comprising two CH2-CH3 Fc domains, and an oligomerization peptide domain. Preferably, the recombinant Fc compound comprises a protein of SEQ ID NO:6 (SEQ ID NO:6 in WO2017172853) or SEQ ID NO:7 (SEQ ID NO:4 in WO2017172853).

In some embodiments the Fc multimers lack any mutation to increase binding affinity of the Fc multimer to a complement system protein. An example of a complement system protein is C1q. In some embodiments, the excluded mutation comprises at least one point mutation in an IgG1 Fc domain of the Fc multimer at any one of positions 267, 268, or 324. In some embodiments, the excluded mutation is at least one of S267E, H268F or S324T. In some embodiments, the excluded mutation comprises at least one mutation at any one of positions 267, 268, and 324, and further comprising at least one point mutation at any one of positions 233, 234, 235, 236, 238, 265, 297, 299, or 328. In some embodiments, the excluded mutation comprises at least one of N297A, T299A, P238D, E233P, G236R, L234V, E233P, L234A, L235A, P238D, D265A, D265W, N297A, N297Q, T299A, or L328F. In some embodiments, the amino acid at position 299 is not mutated from threonine to any other amino acid other than serine or cysteine. In some embodiments, the amino acid at position 298 is not mutated to any amino acid other than proline. In some embodiments, the amino acid at position 236 is not deleted. In a preferred embodiment the excluded mutations are S267E, H268F and S324T. In another preferred embodiment the excluded mutations are S267E, H268F, S324T, and N297A. In another preferred embodiment the excluded mutations are S267E, H268F, S324T, N297A, L234A, and L235A. In another preferred embodiment the excluded mutations are S267E, H268F, S324T, N297A, E233P, L234V, L235A and a deletion of G236. In another preferred embodiment the excluded mutations are S267E, H268F, S324T, L234A, L235A. In another preferred embodiment the excluded mutations are S267E, H268F, S324T, E233P, L234V, L235A, and a deletion of G236. In another preferred embodiment the excluded mutations are S267E, H268F, S324T, and D265A. In another preferred embodiment the excluded mutations are E233P, G236R, S267E, H268F, and S324T. In another preferred embodiment the excluded mutations are G236R, S267E, H268F, S324T. In another preferred embodiment the excluded mutations are E233P, G236R, S267E, H268F, S324T, and L328F. In another preferred embodiment the excluded mutations are P238D, D265G, S267E, H268F, and S324T. In another preferred embodiment the excluded mutations are P238D, D265W, S267E, H268F, and S324T. In another preferred embodiment the excluded mutations are E233P, L234V, L235A, S267E, H268F, N297A, S324T, S328F, and a deletion of G236. In another preferred embodiment the excluded mutations are S267E, H268F, S324T, and L328F. In another preferred embodiment the excluded mutations are P238D, S267E, H268F, and S324T. In another preferred embodiment the excluded mutations are P238D, S267E, H268F, S324T and N297A.

In another alternative embodiment, the recombinant Fc compound used in the present invention is as disclosed in WO2015/168643, WO2017/205436, WO2017/205434 and WO2018/129255, hereby incorporated in their entirety by reference. Preferably, the recombinant Fc compound comprises 2-10 Fc domains, e.g., Fc constructs having 2, 3, 4, 5, 6, 7, 8, 9, or 10 Fc domains. In some embodiments, the recombinant Fc compound comprises 3 Fc domains.

In one aspect, the disclosure features an Fc construct that includes four polypeptides that form three Fc domains. The first polypeptide has the formula A-L-B, wherein A includes a first Fc polypeptide; L is a linker; and B includes a second Fc polypeptide. The second polypeptide has the formula wherein A′ includes a third Fc polypeptide; L′ is a linker; and B′ includes a fourth Fc polypeptide. The third polypeptide includes a fifth Fc polypeptide, and the fourth polypeptide includes a sixth Fc polypeptide. In this aspect, A and A′ combine to form a first Fc domain, B and fifth Fc polypeptide combine to form a second Fc domain, and B′ and sixth Fc polypeptide combine to form a third Fc domain.

In some embodiments of this aspect, A and A′ each include a dimerization selectivity module that promotes dimerization between these Fc polypeptides. In other embodiments, B and the fifth Fc polypeptide each include a dimerization selectivity module that promotes dimerization between these Fc polypeptides. In yet other embodiments, B′ and the sixth Fc polypeptide each include a dimerization selectivity module that promotes dimerization between these Fc polypeptides.

In some embodiments of this aspect, one or more of A, B, A′, B′, the third polypeptide, and the fourth polypeptide consists of an Fc polypeptide. In some embodiments, each of A, B, A′, B′, the third polypeptide, and the fourth polypeptide consists of an Fc polypeptide.

In some embodiments of this aspect, each of B and B′ includes the mutations D399K and K409D, each of A and A′ includes the mutations S354C, T366W, and E357K, and each of the fifth and sixth Fc polypeptides includes the mutations Y349C, T366S, L368A, Y407V, and K370D.

In some embodiments of this aspect, each of A and A′ includes the mutations D399K and K409D, each of B and B′ includes the mutations S354C, T366W, and E357K, and each of the fifth and sixth Fc polypeptides includes the mutations Y349C, T366S, L368A, Y407V, and K370D.

In some embodiments of this aspect, each of L and L′ includes at least 4, 8, 12, 14, 16, 18, or 20 glycines. In some embodiments, each of L and L′ includes 4-30, 8-30, or 12-30 glycines. In some embodiments of this aspect, each of L and L′ comprises, consists of, or consists essentially of of GGGGGGGGGGGGGGGGGGGG (SEQ ID NO:155).

In some embodiments, the Fc construct can further include an IgG CL antibody constant domain and an IgG C_{H}1 antibody constant domain, wherein the IgG CL antibody constant domain is attached to the N-terminus of the IgG CH1 antibody constant domain by way of a linker and the IgG C_{H}1 antibody constant domain is attached to the N-terminus of A, e.g., by way of a linker. In one embodiment, the Fc construct further includes a second IgG CL antibody constant domain and a second IgG C_{H}1 antibody constant domain, wherein the second IgG CL antibody constant domain is attached to the N-terminus of the second IgG C_{H}1 antibody constant domain, e.g., by way of a linker and the second IgG C_{H}1 antibody constant domain is attached to the N-terminus of A′, e.g., by way of a linker.

In some embodiments, the Fc construct further includes a heterologous moiety, e.g., a peptide, e.g., an albumin-binding peptide joined to the N-terminus or C-terminus of B or B′, e.g., by way of a linker.

In other embodiments, the first and second polypeptides of the Fc construct have the same amino acid sequence and the third and fourth polypeptides of the Fc construct have the same amino acid sequence.

In some embodiments, the first and second polypeptide comprise, consist of, or consist essentially of the sequence of SEQ ID NO:120, and the third and fourth polypeptide comprise, consist of, or consist essentially of the sequence of SEQ ID NO:119. In some embodiments of the disclosure, each of the first and second polypeptides comprises, consists of, or consists essentially of the sequence of SEQ ID NO:120 with up to 10 (e.g., up to 9, 8, 7, 6, 5, 4, 3, 2, or 1) single amino acid modifications (e.g., substitutions, e.g., conservative substitutions), and the third and fourth polypeptide comprise, consist of, or consist essentially of the sequence of SEQ ID NO:119 with up to 10 (e.g., up to 9, 8, 7, 6, 5, 4, 3, 2, or 1) single amino acid modifications (e.g., substitutions, e.g., conservative substitutions). In some cases, the first and second polypeptide comprise, consist of, or consist essentially of the sequence of SEQ ID NO:125, and the third and fourth polypeptide comprise, consist of, or consist essentially of the sequence of SEQ ID NO:124. In some embodiments of the disclosure, each of the first and second polypeptides comprises, consists of, or consists essentially of the sequence of SEQ ID NO:125 with up to 10 (e.g., up to 9, 8, 7, 6, 5, 4, 3, 2, or 1) single amino acid modifications (e.g., substitutions, e.g., conservative substitutions), and the third and fourth polypeptide comprise, consist of, or consist essentially of the sequence of SEQ ID NO:124 with up to 10 (e.g., up to 9, 8, 7, 6, 5, 4, 3, 2, or 1) single amino acid modifications (e.g., substitutions, e.g., conservative substitutions). In some embodiments, the first and second polypeptide comprise, consist of, or consist essentially of the sequence of SEQ ID NO:113 or 114, and the third and fourth polypeptide comprise, consist of, or consist essentially of the sequence of SEQ ID NO:107 or 108. In some embodiments of the disclosure, each of the first and second polypeptides comprises, consists of, or consists essentially of the sequence of SEQ ID NO:113 or 114 with up to 10 (e.g., up to 9, 8, 7, 6, 5, 4, 3, 2, or 1) single amino acid modifications (e.g., substitutions, e.g., conservative substitutions), and the third and fourth polypeptide comprise, consist of, or consist essentially of the sequence of SEQ ID NO:107 or 108 with up to 10 (e.g., up to 9, 8, 7, 6, 5, 4, 3, 2, or 1) single amino acid modifications (e.g., substitutions, e.g., conservative substitutions). In some embodiments, the first and second polypeptide comprise, consist of, or consist essentially of the sequence of SEQ ID NO:126, and the third and fourth polypeptide comprise, consist of, or consist essentially of the sequence of SEQ ID NO: 122. In some embodiments of the disclosure, each of the first and second polypeptides comprises, consists of, or consists essentially of the sequence of SEQ ID NO:126 with up to 10 (e.g., up to 9, 8, 7, 6, 5, 4, 3, 2, or 1) single amino acid modifications (e.g., substitutions, e.g., conservative substitutions), and the third and fourth polypeptide comprise, consist of, or consist essentially of the sequence of SEQ ID NO:122 with up to 10 (e.g., up to 9, 8, 7, 6, 5, 4, 3, 2, or 1) single amino acid modifications (e.g., substitutions, e.g., conservative substitutions).

In some embodiments of this aspect of the disclosure, each of the first and third Fc polypeptides includes a complementary dimerization selectivity module that promote dimerization between the first Fc polypeptide and the third Fc polypeptide, and each of the second and fourth Fc polypeptides includes a complementary dimerization selectivity module that promote dimerization between the second Fc polypeptide and the fourth Fc polypeptide. In some embodiments, the complementary dimerization selectivity module of each of the first and second Fc polypeptides includes an engineered protuberance, and the complementary dimerization selectivity module of each of the third and fourth Fc polypeptides includes an engineered cavity.

In some embodiments, one or more of the Fc polypeptides includes an IgG hinge domain or portion thereof, an IgG C_{H}2 antibody constant domain, and an IgG C_{H}3 antibody constant domain. In some embodiments, each of the Fc polypeptides includes an IgG hinge domain or portion thereof, an IgG C_{H}2 antibody constant domain, and an IgG C_{H}3 antibody constant domain. In some embodiments, each of the Fc polypeptides is an IgG1 Fc polypeptide.

In some embodiments of the previous two aspects of the disclosure, the N-terminal Asp in one or more of the first, second, third, and fourth polypeptides is mutated to Gln. In some embodiments, the N-terminal Asp in each of the first, second, third and fourth polypeptides is mutated to Gln.

In some embodiments, one or more of the first, second, third, and fourth polypeptides lack a C-terminal lysine. In some embodiments, each of the first, second, third, and fourth polypeptides lacks a C-terminal lysine.

In some embodiments, the first polypeptide and the second polypeptide have the same amino acid sequence and the third polypeptide and the fourth polypeptide have the same amino acid sequence. In some embodiments, the first polypeptide and the second polypeptide do not have the same amino acid sequence. In some embodiments, the third polypeptide and the fourth polypeptide do not have the same amino acid sequence.

In some embodiments, at least one of the Fc domains includes an amino acid modification that alters one or more of (i) binding affinity to one or more Fc receptors, (ii) effector functions, (iii) the level of Fc domain sulfation, (iv) half-life, (v) protease resistance, (vi) Fc domain stability, and/or (vii) susceptibility to degradation. In some embodiments, the Fc domain includes an amino acid modification that alters binding affinity to one or more Fc receptors, e.g., S267E/L328F. In some embodiments, the Fc receptor is FcγRIIb. In some cases, the modification described herein increases affinity to the FcγRIIb receptor. In some cases, the S267E/L328F modification increases binding affinity to FcγRIIb. In some embodiments, the Fc domain includes an amino acid modification that alters the level of Fc domain sulfation, e.g., 241F, 243F, 246K, 260T, or 301R. In some embodiments, the Fc domain includes an amino acid modification that alters protease resistance, e.g., selected from the following sets: 233P, 234V, 235A, and 236del; 237A, 239D, and 332E; 237D, 239D, and 332E; 237P, 239D, and 332E; 237Q, 239D, and 332E; 237S, 239D, and 332E; 239D, 268F, 324T, and 332E; 239D, 326A, and 333A; 239D and 332E; 243L, 292P, and 300L; 267E, 268F, 324T, and 332E; 267E and 332E; 268F, 324T, and 332E; 326A, 332E, and 333A; or 326A and 333A. In some embodiments, the Fc domain includes an amino acid modification that alters Fc domain susceptibility to degradation, e.g., C233X, D234X, K235X, S236X, T236X, H237X, C239X, S241X, and G249X, wherein X is any amino acid.

In some embodiments, the disclosure features an Fc construct including a) a first polypeptide including i) a first Fc polypeptide; ii) a second Fc polypeptide; and iii) a linker joining the first Fc polypeptide to the second Fc polypeptide; b) a second polypeptide including i) a third Fc polypeptide; ii) a fourth Fc polypeptide; and iii) a linker joining the third Fc polypeptide to the fourth Fc polypeptide; c) a third polypeptide includes a fifth Fc polypeptide; and d) a fourth polypeptide includes a sixth Fc polypeptide; wherein the first Fc polypeptide and fifth Fc polypeptide combine to form a first Fc domain, the second Fc polypeptide and fourth Fc polypeptide combine to form a second Fc domain, and the third Fc polypeptide and sixth Fc polypeptide combine to form a third Fc domain, and wherein at least one Fc domain includes an amino acid modification at position I253 (e.g., a single amino acid modification at position I253).

In some embodiments, the first and second polypeptides are identical to each other and the third and fourth polypeptides are identical to each other. In some embodiments, the first Fc domain includes an amino acid modification at position I253. In some cases, one or both of the first and fifth Fc polypeptides comprises an amino acid substitution at position I253. In some embodiments, the second Fc domain includes an amino acid modification at position I253. In some embodiments, one or both of the second and fourth Fc domain monomers comprises an amino acid substitution at position I253. In some embodiments, the third Fc domain includes an amino acid modification at position I253. In some embodiments, one or both of the third and sixth Fc polypeptides comprises an amino acid substitution at position I253. In some embodiments, each amino acid modification (e.g., substitution) at position I253 is independently selected from the group consisting of I253A, I253C, I253D, I253E, I253F, I253G, I253H, I253I, I253K, I253L, I253M, I253N, I253P, I253Q, I253R, I253S, I253T, I253V, I253W, and I253Y. In some embodiments, each amino acid modification (e.g., substitution) at position I253 is I253A.

In another aspect, the disclosure features an Fc construct including: a) a first polypeptide including: i). a first Fc polypeptide; ii). a second Fc polypeptide; and iii). a linker joining the first Fc polypeptide to the second Fc polypeptide; b). a second polypeptide including i). a third Fc polypeptide; ii). a fourth Fc polypeptide; and iii). a linker joining the third Fc polypeptide to the fourth Fc polypeptide; c). a third polypeptide includes a fifth Fc polypeptide; and d). a fourth polypeptide includes a sixth Fc polypeptide; wherein the first Fc polypeptide and fifth Fc polypeptide combine to form a first Fc domain, the second Fc polypeptide and fourth Fc polypeptide combine to form a second Fc domain, and the third Fc polypeptide and sixth Fc polypeptide combine to form a third Fc domain, and wherein at least one Fc domain comprises an amino acid modification at position R292 (e.g., a single amino acid modification).

In some embodiments, the first Fc domain includes an amino acid modification at position R292. In some embodiments, one or both of the first and the fifth Fc polypeptides comprises an amino acid substitution at position R292. In some embodiments, the second Fc domain includes an amino acid modification at position R292. In some embodiments, one or both of the second and the fourth Fc polypeptides comprises an amino acid substitution at position R292. In some embodiments, the third Fc domain includes an amino acid modification at position R292. In some embodiments, one or both of the third and the sixth Fc polypeptides comprises an amino acid substitution at position R292. In some embodiments, each of the first, second, and third Fc domain includes an amino acid modification (e.g., substitution) at position R292. In some embodiments, each of the first, second, and third Fc domain includes the amino acid modification (e.g., substitution) R292P (i.e., each Fc monomer has R292P modification). In some embodiments, one or both of the first and fifth Fc polypeptides includes the amino acid substitution R292P, one or both of the second and fourth Fc polypeptides includes amino acid substitution R292P, and one or both of the third and sixth Fc polypeptides includes the amino acid substitution R292P.

In some embodiments, each amino acid modification (e.g., substitution) at position R292 is independently selected from R292D, R292E, R292L, R292P, R292Q, R292R, R292T, or R292Y. In some embodiments, each amino acid modification (e.g., substitution) at position R292 is R292P. In some embodiments, each of the first and third Fc domain includes the amino acid modification (e.g., substitution) I253A, and each of the first, second, and third Fc domain includes the amino acid modification (e.g., substitution) R292P. In some embodiments, one or both of the first and fifth Fc polypeptides includes the amino acid substitution I253A, one or both of the third and sixth Fc polypeptides includes the amino acid substitution I253A, one or both of the first and fifth Fc polypeptides includes the amino acid substitution R292P, one or both of the second and fourth Fc polypeptides includes amino acid substitution R292P, and one or both of the third and sixth Fc polypeptides includes the amino acid substitution R292P. In some embodiments, each of the first, second, and third Fc domain includes the amino acid modification (e.g., substitution) I253A and R292P. In some embodiments, one or both of the first and fifth Fc polypeptides includes the amino acid substitution I253A, one or both of the second and fourth Fc polypeptides includes amino acid substitution I253A, and one or both of the third and sixth Fc polypeptides includes the amino acid substitution I253A, one or both of the first and fifth Fc polypeptides includes the amino acid substitution R292P, one or both of the second and fourth Fc polypeptides includes amino acid substitution R292P, and one or both of the third and sixth Fc polypeptides includes the amino acid substitution R292P.

In some embodiments, each of the first Fc domain and third Fc domain include the amino acid substitutions I253A and R292P, and the second Fc domain includes the amino acid substitution R292P. In some cases, one or both of the first and fifth Fc polypeptides comprises the amino acid substitution I253A; one or both of the first and fifth Fc polypeptides comprises the amino acid substitution R292P; one or both of the third and sixth Fc polypeptides comprises the amino acid substitution I253A; one or both of the third and sixth Fc polypeptides comprises the amino acid substitution R292P; and one or both of the second and fourth Fc polypeptides comprises the amino acid substitution R292P.

In some embodiments, the second Fc domain includes the amino acid substitution I253A. In some embodiments, one or both of the second and fourth Fc polypeptides comprises the amino acid substitution I253A. In some embodiments, each of the first Fc domain and third Fc domain include the amino acid substitution I253A. In some embodiments, one or both of the first and fifth Fc polypeptides comprises the amino acid substitution I253A and one or both of the third and sixth Fc polypeptides comprises the amino acid substitution I253A. In some embodiments, each of the first Fc domain, second Fc domain, and third Fc domain include the amino acid substitution I253A. In some embodiments, one or both of the first and fifth Fc polypeptides comprises the amino acid substitution I253A, one or both of the second and fourth Fc polypeptides comprises the amino acid substitution I253A, and one or both of the third and sixth Fc polypeptides comprises the amino acid substitution I253A.

In some embodiments, the second Fc domain includes the amino acid substitution R292P. In some embodiments, one or both of the second and fourth Fc polypeptides comprises the amino acid substitution R292P. In some embodiments, the second Fc domain includes the amino acid substitutions I253A and R292P. In some embodiments, one or both of the second and fourth Fc polypeptides comprises the amino acid substitution I253A, and one or both of the second and fourth Fc polypeptides comprises the amino acid substitution R292P. In some embodiments, each of the first Fc domain and third Fc domain include the amino acid substitution I253A, and the second Fc domain includes the amino acid substitution R292P. In some embodiments, one or both of the first and fifth Fc polypeptides comprises the amino acid substitution I253A; one or both of the third and sixth Fc polypeptides comprises the amino acid substitution I253A; and one or both of the second and fourth Fc polypeptides comprises the amino acid substitution R292P.

In some embodiments, each of the first Fc domain and third Fc domain include the amino acid substitution I253A, and the second Fc domain includes the amino acid substitution I253A and R292P. In some embodiments, one or both of the first and fifth Fc polypeptides comprises the amino acid substitution I253A; one or both of the third and sixth Fc polypeptides comprises the amino acid substitution I253A; one or both of the second and fourth Fc polypeptides comprises the amino acid substitution I253A; and one or both of the second and fourth Fc polypeptides comprises the amino acid substitution R292P. In some embodiments, each of the first Fc domain and third Fc domain include the amino acid substitution R292P. In some embodiments, one or both of the first and fifth Fc polypeptides comprises the amino acid substitution R292P and one or both of the third and sixth Fc polypeptides comprises the amino acid substitution R292P.

In some embodiments, the first Fc domain and third Fc domain include the amino acid substitution R292P, and the second Fc domain includes the amino acid substitution I253A. In some embodiments, one or both of the first and fifth Fc polypeptides comprises the amino acid substitution R292P; one or both of the third and sixth Fc polypeptides comprises the amino acid substitution R292P; and one or both of the second and fourth Fc polypeptides comprises the amino acid substitution I253A. In some embodiments, each of the first Fc domain and third Fc domain include I253A and R292P (e.g., include the amino acid substitutions I253A and R292P). In some embodiments, one or both of the first and fifth Fc polypeptides comprises the amino acid substitution I253A; one or both of the first and fifth Fc polypeptides comprises the amino acid substitution R292P; one or both of the third and sixth Fc polypeptides comprises the amino acid substitution I253A; and one or both of the third and sixth Fc polypeptides comprises the amino acid substitution R292P.

In some embodiments, each of the first Fc domain and third Fc domain include the amino acid substitutions I253A and R292P, and the second Fc domain includes the amino acid substitution I253A. In some embodiments, one or both of the first and fifth Fc polypeptides comprises the amino acid substitution I253A; one or both of the first and fifth Fc polypeptides comprises the amino acid substitution R292P; one or both of the third and sixth Fc polypeptides comprises the amino acid substitution I253A; one or both of the third and sixth Fc polypeptides comprises the amino acid substitution R292P; and one or both of the second and fourth Fc polypeptides comprises the amino acid substitution I253A. In some embodiments, each of the first Fc domain, second Fc domain, and third Fc domain include the amino acid substitution R292P. In some embodiments, one or both of the first and fifth Fc polypeptides comprises the amino acid substitution R292P; one or both of the second and fourth Fc polypeptides comprises the amino acid substitution R292P; and one or both of the third and sixth Fc polypeptides comprises the amino acid substitution R292P.

In some embodiments, each of the first Fc domain and third Fc domain include the amino acid substitution R292P, and the second Fc domain includes the amino acid substitutions I253A and R292P. In some embodiments, one or both of the first and fifth Fc polypeptides comprises the amino acid substitution R292P; one or both of the third and sixth Fc polypeptides comprises the amino acid substitution R292P; one or both of the second and fourth Fc polypeptides comprises the amino acid substitution I253A; and one or both of the second and fourth Fc polypeptides comprises the amino acid substitution R292P. In some embodiments, each of the first Fc domain, second Fc domain, and third Fc domain include the amino acid substitutions I253A and R292P. In some embodiments, one or both of the first and fifth Fc polypeptides comprises the amino acid substitution I253A; one or both of the first and fifth Fc polypeptides comprises the amino acid substitution R292P; one or both of the second and fourth Fc polypeptides comprises the amino acid substitution I253A; one or both of the second and fourth Fc polypeptides comprises the amino acid substitution R292P; one or both of the third and sixth Fc polypeptides comprises the amino acid substitution I253A; and one or both of the third and sixth Fc polypeptides comprises the amino acid substitution R292P.

In some embodiments, each of the first, second, and third Fc domains include the amino acid substitution R292P. In some embodiments, one or both of the first and fifth Fc polypeptides comprises the amino acid substitution R292P. In some embodiments, one or both of the third and sixth Fc polypeptides comprises the amino acid substitution R292P. In some embodiments, one or both of the second and fourth Fc polypeptides comprises the amino acid substitution R292P.

In some embodiments, the Fc constructs described herein do not include an antigen-recognition region, e.g., a variable domain or a complementarity determining region (CDR). In some embodiments, the Fc construct (or an Fc domain within an Fc construct) is formed entirely or in part by association of Fc polypeptides that are present in different polypeptides. In certain embodiments, the Fc construct does not include an additional domain (e.g., an IgM tailpiece or an IgA tailpiece) that promotes association of two polypeptides. In other embodiments, covalent linkages (e.g., disulfide bridges) are present only between two Fc polypeptides that join to form an Fc domain. In other embodiments, the Fc construct does not include covalent linkages (e.g., disulfide bridges) between Fc domains. In still other embodiments, the Fc construct provides for sufficient structural flexibility such that all or substantially all of the Fc domains in the Fc construct are capable of simultaneously interacting with an Fc receptor on a cell surface. In one embodiment, the Fc polypeptides are different in primary sequence from wild-type or from each other in that they have dimerization selectivity modules.

In another aspect, the disclosure features compositions and methods for promoting selective dimerization of Fc polypeptides. The disclosure includes an Fc domain wherein the two Fc polypeptides of the Fc domain include identical mutations in at least two positions within the ring of charged residues at the interface between C_{H}3 antibody constant domains. The disclosure also includes a method of making such an Fc domain, including introducing complementary dimerization selectivity modules having identical mutations in two Fc polypeptide sequences in at least two positions within the ring of charged residues at the interface between C_{H}3 antibody constant domains. The interface between C_{H}3 antibody constant domains consists of a hydrophobic patch surrounded by a ring of charged residues. When one C_{H}3 antibody constant domain comes together with another, these charged residues pair with residues of the opposite charge. By reversing the charge of both members of two or more complementary pairs of residues, mutated Fc polypeptides remain complementary to Fc polypeptides of the same mutated sequence, but have a lower complementarity to Fc polypeptides without those mutations. In this embodiment, the identical dimerization selectivity modules promotes homodimerization. Such Fc domains include Fc polypeptides containing the double mutants K409D/D399K, K392D/D399K, E357K/K370E, D356K/K439D, K409E/D399K, K392E/D399K, E357K/K370D, or D356K/K439E. In another embodiment, an Fc domain includes Fc polypeptides including quadruple mutants combining any pair of the double mutants, e.g., K409D/D399K/E357K/K370E.

In another embodiment, in addition to the identical dimerization selectivity modules, the Fc polypeptides of the Fc domain include complementary dimerization selectivity modules having non-identical mutations that promote specific association (e.g., engineered cavity and protuberance). As a result, the two Fc polypeptides include two dimerization selectivity modules and remain complementary to each other, but have a decreased complementarity to other Fc polypeptides. This embodiment promotes heterodimerization between a cavity-containing Fc polypeptide and a protuberance-containing Fc polypeptide. In one example, the complementary dimerization selectivity modules having non-identical mutations in charged pair residues of both Fc polypeptides are combined with a protuberance on one Fc polypeptide and a cavity on the other Fc polypeptide. In another embodiment, the Fc polypeptides of the Fc domain include complementary dimerization selectivity modules having non-identical mutations that promote specific association (e.g., engineered cavity and protuberance), and do not include the identical dimerization selectivity modules.

In any of the Fc constructs described herein, it is understood that the order of the Fc polypeptides is interchangeable. For example, in a polypeptide having the formula A-L-B, the carboxy terminus of A can be joined to the amino terminus of L, which in turn is joined at its carboxy terminus to the amino terminus of B. Alternatively, the carboxy terminus of B can be joined to the amino terminus of L, which in turn is joined at its carboxy terminus to the amino terminus of C. Both of these configurations are encompassed by the formula A-L-B.

The properties of these constructs allow for the efficient generation of substantially homogenous compositions. The degree of homogeneity of a composition influences the pharmacokinetics and in vivo performance of the composition. Such homogeneity in a composition is desirable in order to ensure the safety, efficacy, uniformity, and reliability of the composition. An Fc construct of the disclosure can be in a population or composition that is substantially homogenous (e.g., at least 85%, 90%, 95%, 98%, or 99% homogeneous).

As described in further detail herein, the disclosure features substantially homogenous compositions containing Fc constructs that all have the same number of Fc domains, as well as methods of preparing such substantially homogenous compositions.

An Fc construct of the disclosure can be in a pharmaceutical composition that includes a substantially homogenous population (e.g., at least 85%, 90%, 95%, 98%, or 99% homogeneous) of the Fc construct having 2-10 Fc domains (e.g., 2-8 Fc domains, 2-6 Fc domains, 2-4 Fc domains, 2-3 Fc domains, 3-5 Fc domains, or 5-10 Fc domains) e.g., a construct having 2, 3, 4, 5, 6, 7, 8, 9, or 10 Fc domains, such as those described herein. Consequently, pharmaceutical compositions can be produced that do not have substantial aggregation or unwanted multimerization of Fc constructs.

Polynucleotides

The disclosure further relates to a polynucleotide encoding an Fc fusion polypeptide or a polypeptide for an Fc multimer. The term “polynucleotide(s)” generally refers to any polyribonucleotide or polydeoxyribonucleotide that may be unmodified RNA or DNA or modified RNA or DNA. The polynucleotide can be single- or double-stranded DNA, single or double-stranded RNA. As used herein, the term “polynucleotide(s)” also includes DNAs or RNAs that comprise one or more modified bases and/or unusual bases, such as inosine. It will be appreciated that a variety of modifications may be made to DNA and RNA that serve many useful purposes known to those of skill in the art. The term “polynucleotide(s)” as it is employed herein embraces such chemically, enzymatically, or metabolically modified forms of polynucleotides, as well as the chemical forms of DNA and RNA characteristic of viruses and cells, including, for example, simple and complex cells.

The skilled person would understand that, due to the degeneracy of the genetic code, a given polypeptide can be encoded by different polynucleotides. These “variants” are encompassed by the Fc multimers disclosed herein.

The polynucleotides of the Fc multimers may be an isolated polynucleotide. The term “isolated” polynucleotide refers to a polynucleotide that is substantially free from other nucleic acid sequences, such as and not limited to other chromosomal and extrachromosomal DNA and RNA. In one embodiment, the isolated polynucleotides are purified from a host cell. Conventional nucleic acid purification methods known to skilled artisans may be used to obtain isolated polynucleotides. The term also includes recombinant polynucleotides and chemically synthesized polynucleotides.

Another aspect of the disclosure is a plasmid or vector comprising a polynucleotide according to the disclosure. In one embodiment, as disclosed in WO2017/129737, the plasmid or vector comprises an expression vector. In one embodiment, the vector is a transfer vector for use in human gene therapy. Another aspect of the disclosure is a host cell comprising a polynucleotide, a plasmid, or vector of the disclosure.

The host cell of the disclosure is employed in a method of producing an Fc multimer. The method comprises:
(a) culturing host cells of the disclosure under conditions such that the desired insertion protein is expressed; and
(b) optionally recovering the desired insertion protein from the host cells or from the culture medium.

In a separate embodiment, the Fc multimers are purified to ≥80% purity, ≥90% purity, ≥95% purity, ≥99% purity, or ≥99.9% purity with respect to contaminating macromolecules, for example other proteins and nucleic acids, and free of infectious and pyrogenic agents. An isolated Fc multimer of the disclosure may be substantially free of other, non-related polypeptides.

In certain embodiments of the present invention, the Fc multimers are those described in WO2014/060712. Examples include polymeric proteins comprising five, six or seven polypeptide monomer units, wherein each polypeptide monomer unit comprises an Fc receptor binding portion comprising two immunoglobulin G heavy chain constant regions, wherein each immunoglobulin G heavy chain constant region comprises a cysteine residue which is linked via a disulfide bond to a cysteine residue of an immunoglobulin G heavy chain constant region of an adjacent polypeptide monomer unit, wherein the polymeric protein does not comprise a further immunomodulatory portion or an antigen portion that causes antigen-specific immunosuppression when administered to a mammalian subject. In certain aspects, the two immunoglobulin G heavy chain constant regions are linked via a polypeptide linker as a single chain Fc. In other aspects, the polypeptide monomer unit consists of an Fc receptor binding portion and a tailpiece region fused to the two immunoglobulin G heavy chain constant regions, which facilitates assembly of the monomer units into a polymer.

In another embodiment, each of the immunoglobulin G heavy chain constant regions comprises an amino acid sequence of a mammalian heavy chain constant region, preferably a human heavy chain constant region; or variant thereof. A suitable human IgG subtype is IgG1.

The Fc receptor binding portion may comprise more than the Fc portion of an immunoglobulin. For example, as described in WO2014/060712, it may include the hinge region of the immunoglobulin which occurs between CH1 and CH2 domains in a native immunoglobulin. For certain immunoglobulins, the hinge region is necessary for binding to Fc receptors. Preferably, the Fc receptor binding portion lacks a CH1 domain and heavy chain variable region domain (VH). The Fc receptor binding portion may be truncated at the C- and/or N-terminus compared to the Fc portion of the corresponding immunoglobulin. The polymeric protein is formed by virtue of each immunoglobulin G heavy chain constant region comprising a cysteine residue which is linked via a disulfide bond to a cysteine residue of an immunoglobulin G heavy chain constant region of an adjacent polypeptide monomer unit. The ability of monomer units based on IgG heavy chain constant regions to form polymers may be improved by modifying the parts of the IgG heavy chain constant regions to be more like the corresponding parts of IgM or IgA. Each of the immunoglobulin heavy chain constant regions or variants thereof is an IgG heavy chain constant region comprising an amino acid sequence which comprises a cysteine residue at position 309 and, preferably, a leucine residue at position 310.

For the aspects of the invention where a tailpiece region is present, each polypeptide monomer unit comprises a tailpiece region fused to each of the two immunoglobulin G heavy chain constant regions, wherein the tailpiece region of each polypeptide monomer unit facilitates the assembly of the monomer units into a polymer such as described in WO2014/060712. For example, the tailpiece region is fused C-terminal to each of the two immunoglobulin heavy chain constant regions. The tailpiece region can be an IgM or IgA tailpiece, or fragment or variant thereof.

In one embodiment, an intervening amino acid sequence may be provided between the heavy chain constant region and the tailpiece, or the tailpiece may be fused directly to the C-terminus of the heavy chain constant region such as disclosed in WO2014/060712. For example, a short linker sequence may be provided between the tailpiece region and immunoglobulin heavy chain constant region. Typical linker sequences are of between 1 and 20 amino acids in length, typically 2, 3, 4, 5, 6 or up to 8, 10, 12, or 16 amino acids in length. A suitable linker to include between the heavy chain region and tailpiece region encodes for Leu-Val-Leu-Gly (SEQ ID NO: 8). A preferred tailpiece region is the tailpiece region of human IgM, which is PTLYNVSLVMSDTAGTCY (SEQ ID NO: 9) (Rabbitts T H et al, 1981. Nucleic Acids Res. 9 (18), 4509-4524; Smith et al (1995) J Immunol 154: 2226-2236). This tailpiece may be modified at the N-terminus by substituting Pro for the initial Thr. This does not affect the ability of the tailpiece to promote polymerization of the monomer. Further suitable variants of the human IgM tailpiece are described in Sorensen et al (1996) J Immunol 156: 2858-2865. A further IgM tailpiece sequence is GKPTLYNVSLIMSDTGGTCY (SEQ ID NO: 10) from rodents. An alternative preferred tailpiece region is the tailpiece region of human IgA, which is PTHVNVSVVMAEVDGTCY (SEQ ID NO:11). Other suitable tailpieces from IgM or IgA of other species, or even synthetic sequences which facilitate assembly of the monomer units into a polymer, may be used. It is not necessary to use an immunoglobulin tailpiece from the same species from which the immunoglobulin heavy chain constant regions are derived, although it is preferred to do so.

In certain aspects, the polymeric protein does not activate the classical pathway of complement, although it may be capable of binding to C1q. The polymeric protein typically has a diameter of about 20 nm, such as from 15 to 25 nm or up to 30 nm. As a consequence of the molecular size and diameter, the polymeric protein typically has a good degree of tissue penetration.

The preferred Fc multimer described in WO2014/060712 is the hexamer of SEQ ID NO:12 (SEQ ID NO:8 in WO2014/060712), from which the signal peptide is cleaved off during secretion so that the mature product comprises residues 21 to 269 of SEQ ID NO:12.

In certain embodiments of the present invention, the Fc multimers used are those described in WO2015/132364, which relates to multimeric fusion proteins which bind to human Fc receptors. Fusion proteins comprise a tailpiece, in the absence of a cysteine residue at position 309.

In one embodiment, the multimeric fusion proteins comprise two or more polypeptide monomer units, wherein each polypeptide monomer unit comprises an antibody Fc-domain comprising two heavy chain Fc-regions. Each heavy chain Fc-region comprises any amino acid residue other than cysteine at position 309, and is fused at its C-terminal to a tailpiece which causes the monomer units to assemble into a multimer. Each polypeptide monomer unit does not comprise an antibody variable region.

In certain aspects, the multimeric fusion proteins further comprise a fusion partner, which can be an antigen, pathogen-associated molecular pattern (PAMP), drug, ligand, receptor, cytokine or chemokine. The fusion partner is fused to the N-terminus of each heavy chain Fc-region either directly or indirectly by means of an intervening amino acid sequence, such as a hinge. A short linker sequence, alternatively, may be provided between the fusion partner and the heavy chain Fc-region.

In other aspects, the multimeric fusion proteins do not comprise one or more antibody variable regions. Typically, the molecules do not comprise either a VH or a VL antibody variable region. In certain further aspects, the multimeric fusion proteins of WO2015/132364 do not comprise a Fab fragment.

In another embodiment, each polypeptide monomer unit of the multimeric fusion protein comprises an antibody Fc-domain, which may be derived from any suitable species, including humans, for instance. In addition, the antibody Fc-domain may be derived from any suitable class of antibody, including IgA (including subclasses IgA1 and IgA2), IgD, IgE, IgG (including subclasses IgG1, IgG2, IgG3 and IgG4), and IgM.

The antibody Fc-domain comprises two Fc polypeptide chains, each referred to as a heavy chain Fc-region. The two heavy chain Fc regions dimerize to create the antibody Fc-domain. The two heavy chain Fc regions within the antibody Fc domain may be different from one another but will typically be the same.

Typically, each heavy chain Fc-region comprises or consists of two or three heavy chain constant domains. IgA, IgD and IgG, for instance, are composed of two heavy chain constant domains (CH2 and CH3) while IgE and IgM are composed of three heavy chain constant domains (CH2, CH3 and CH4). The heavy chain Fc-regions may comprise heavy chain constant domains from one or more different classes of antibody, for example one, two or three different classes.

Thus, the heavy chain Fc region in the Fc multimer used in one embodiment of the present invention comprises a CH3 domain derived from IgG1 such as disclosed in WO2015/132364. In a separate embodiment, the heavy chain Fc region comprises a CH2 domain derived from IgG4 and a CH3 domain derived from IgG1. In certain embodiments, the heavy chain Fc region comprises an arginine residue at position 355. In other embodiments, the heavy chain Fc region comprises a cysteine residue at position 355.

The heavy chain Fc-region in the Fc multimer used in one embodiment of the present invention comprises a CH4 domain from IgM. The IgM CH4 domain is typically located between the CH3 domain and the tailpiece.

In other aspects, the heavy chain Fc-region comprises CH2 and CH3 domains derived from IgG and a CH4 domain derived from IgM.

The tailpiece of the multimeric fusion proteins may comprise any suitable amino acid sequence. It may be a tailpiece found in a naturally occurring antibody, or alternatively, it may be a modified tailpiece which differs in length and/or composition from a natural tailpiece. Other modified tailpieces may be entirely synthetic and may be designed to possess desired properties for multimerization, such as length, flexibility and cysteine composition. The tailpiece may be derived from any suitable species, including humans.

The tailpiece may comprise all or part of an 18 amino acid tailpiece sequence from human IgM or IgA as shown in SEQ ID NO:9 or SEQ ID NO:11.

The tailpiece may be fused directly to the C-terminus of the heavy chain Fc-region, or, alternatively, indirectly by means of an intervening amino acid sequence. A short linker sequence, for instance, may be provided between the tailpiece and the heavy chain Fc-region.

The tailpiece may include variants or fragments of the native sequences described above. A variant of an IgM or IgA tailpiece typically has an amino acid sequence which is identical to the native sequence in 8, 9, 10, 11, 12, 13, 14, 15, 16, or 17 of the 18 amino acid positions. A fragment typically comprises 8, 9, 10, 11, 12, 13, 14, 15, 16, or 17 amino acids. The tailpiece may be a hybrid IgM/IgA tailpiece.

Each heavy chain Fc-region in the Fc multimer used in an embodiment of the present invention may, optionally, possess a native or a modified hinge region at its N-terminus. The types of modified hinge regions that can be incorporated in the Fc multimers used in the present invention are disclosed in WO2015/132364. For example, the heavy chain Fc-region possesses an intact hinge region at its N-terminus. In certain aspects, as disclosed in WO2015/132364, the heavy chain Fc-region and hinge region are derived from IgG4 and the hinge region comprises the mutated sequence CPPC (SEQ ID NO:13).

Examples of suitable hinge sequences are shown in SEQ ID Nos:13 to 35.

For example, the multimeric fusion proteins may comprise two, three, four, five, six, seven, eight, nine, ten, eleven or twelve or more polypeptide monomer units. In addition, the multimeric fusion protein may comprise a mixture of multimeric fusion proteins of different sizes, having a range of numbers of polypeptide monomer units.

Accordingly, in a specific embodiment, a multimeric fusion protein used in the present invention consists of six polypeptide monomer units, wherein each polypeptide monomer unit consists of an antibody Fc-domain and a tailpiece region, wherein each antibody Fc domain consists of two heavy chain Fc-regions in which the amino acid residue at position 309 is any amino acid residue other than cysteine, and, optionally, each heavy chain Fc region possesses a hinge region at the N-terminus, and wherein the tailpiece region is fused to the C-terminus of each heavy chain Fc region and causes the monomer units to assemble into a multimer.

Similarly, the polypeptide monomer units within a particular multimeric fusion protein may be the same as one another or different from one another.

In certain embodiments, a polypeptide chain of a polypeptide monomer unit comprises an amino acid sequence as provided in SEQ ID NOs:36 to 57, optionally with an alternative hinge or tailpiece sequence.

In another example, a multimeric fusion protein used in the present invention comprises or consists of two or more, preferably six, polypeptide monomer units, wherein each polypeptide monomer unit comprises two identical polypeptide chains, each polypeptide chain comprising or consisting of the sequence given in any one of the above SEQ ID NOs:36 to 57 (SEQ ID NOs:26 to 47 of WO2015/132364), and wherein each polypeptide monomer unit does not comprise an antibody variable region.

In certain embodiments, the multimeric fusion proteins comprise one or more mutations which decrease cytokine release and/or decrease platelet activation and/or decrease C1q binding and/or increase the potency of inhibition of macrophage phagocytosis of antibody-coated target cells and/or alter binding to one or more Fc-receptors when compared to unmodified multimeric fusion proteins.

In certain embodiments of the present invention, the Fc multimers used are those described in WO2015/132365, which relates to multimeric fusion proteins which bind to human Fc receptors.

The multimeric fusion proteins used in an embodiment of the present invention comprise two or more polypeptide monomer units, wherein each polypeptide monomer unit comprises an antibody Fc-domain comprising two heavy chain Fc-regions such as those disclosed in WO2015/132365. Each heavy chain Fc-region comprises a cysteine residue at position 309, and at least one further mutation which alters FcR binding, and is fused at its C-terminus to a tailpiece which causes the monomer units to assemble into a multimer. Each polypeptide monomer unit does not comprise an antibody variable region.

In certain aspects, the multimeric fusion proteins further comprise a fusion partner, as described above. In other aspects, the multimeric fusion proteins do not comprise one or more antibody variable regions or a Fab fragment, as described above. In one embodiment, each polypeptide monomer unit of the multimeric fusion protein comprises an antibody Fc-domain with heavy chain Fc regions, as described above. The tailpieces, modified hinge regions, and polypeptide monomer units of the multimeric fusion proteins of the present invention comprise the features described above.

The multimeric fusion protein used in a specific embodiment of the present invention consists of six polypeptide monomer units, wherein each polypeptide monomer unit consists of an antibody Fc-domain and a tailpiece region, wherein each antibody Fc domain consists of two heavy chain Fc-regions in which the amino acid residue at position 309 in each heavy chain Fc region is a cysteine residue and each heavy chain Fc region comprises at least one further mutation which alters FcR binding and, optionally, each heavy chain Fc region possesses a hinge region at the N-terminus, and wherein the tailpiece region is fused to the C-terminus of each heavy chain Fc region and causes the monomer units to assemble into a multimer.

In certain embodiments, the polypeptide chains of polypeptide monomer units comprise amino acid sequences as described above.

In another example, a multimeric fusion protein comprises or consists of two or more, preferably six, polypeptide monomer units, wherein each polypeptide monomer unit comprises two identical polypeptide chains each polypeptide chain comprising or consisting of the sequence given in any one of the SEQ ID NOs:58 to 94 (corresponding to SEQ ID NOs:26 to 32 and 50 to 64 of WO2015/132365), and wherein each polypeptide monomer unit does not comprise an antibody variable region.

In certain embodiments, as taught in WO2015/132365, the multimeric fusion proteins used in the invention comprise one or more mutations which enable such functions as described above.

The various products of the disclosure are useful as medicaments. Accordingly, the disclosure relates to a pharmaceutical composition comprising an Fc multimer, a polynucleotide of the disclosure, or a plasmid or vector of the disclosure.

An aspect of the invention is a method of treating an immune complex-mediated kidney disorder in a subject in need thereof. The method comprises administering to said subject a therapeutically effective amount of the Fc multimer. In another embodiment, the method comprises administering to said subject a therapeutically effective amount of a polynucleotide of the disclosure or a plasmid or vector of the disclosure.

Expression of the Proposed Fc Multimers

The production of recombinant proteins at high levels in suitable host cells requires the assembly of the above-mentioned modified cDNAs into efficient transcriptional units together with suitable regulatory elements in a recombinant expression vector that can be propagated in various expression systems according to methods known to those skilled in the art. Efficient transcriptional regulatory elements could be derived from viruses having animal cells as their natural hosts or from the chromosomal DNA of animal cells. For example, promoter-enhancer combinations derived from the Simian Virus 40, adenovirus, BK polyoma virus, human cytomegalovirus, or the long terminal repeat of Rous sarcoma virus, or promoter-enhancer combinations including strongly constitutively transcribed genes in animal cells like beta-actin or GRP78 can be used. In order to achieve stable high levels of mRNA transcribed from the cDNAs, the transcriptional unit should contain in its 3′-proximal part a DNA region encoding a transcriptional termination-polyadenylation sequence. For example, this sequence can be derived from the Simian Virus 40 early transcriptional region, the rabbit beta globin gene, or the human tissue plasminogen activator gene.

The cDNAs can then be integrated into the genome of a suitable host cell line for expression of the Fc multimer. In some embodiments, this cell line should be an animal cell-line of vertebrate origin in order to ensure correct folding, disulfide bond formation, asparagine-linked glycosylation and other post-translational modifications as well as secretion into the cultivation medium. Examples of other post-translational modifications are tyrosine 0-sulfation and proteolytic processing of the nascent polypeptide chain. Examples of cell lines that can be used are monkey COS-cells, mouse L-cells, mouse C127-cells, hamster BHK-21 cells, human embryonic kidney 293 cells, and hamster CHO-cells.

The recombinant expression vector encoding the corresponding cDNAs can be introduced into an animal cell line in several different ways. For example, recombinant expression vectors can be created from vectors based on different animal viruses. Examples of these are vectors based on baculovirus, vaccinia virus, adenovirus, and bovine papilloma virus.

The transcription units encoding the corresponding DNAs can also be introduced into animal cells together with another recombinant gene which may function as a dominant selectable marker in these cells in order to facilitate the isolation of specific cell clones which have integrated the recombinant DNA into their genome. Examples of this type of dominant selectable marker genes are TN4 amino glycoside phosphotransferase, conferring resistance to geneticin (G418), hygromycin phosphotransferase, conferring resistance to hygromycin, and puromycin acetyl transferase, conferring resistance to puromycin. The recombinant expression vector encoding such a selectable marker can reside either on the same vector as the one encoding the cDNA of the desired protein, or it can be encoded on a separate vector which is simultaneously introduced and integrated to the genome of the host cell, frequently resulting in a tight physical linkage between the different transcription units.

Other types of selectable marker genes which can be used together with the cDNA of the desired protein are based on various transcription units encoding dihydrofolate reductase (dhfr). After introduction of this type of gene into cells lacking endogenous dhfr-activity, for example CHO-cells (DUKX-B11, DG-44), it will enable these to grow in media lacking nucleosides. An example of such a medium is Ham's F12 without hypoxanthine, thymidine, and glycine. These dhfr-genes can be introduced together with the cDNA encoding the IgG Fc fusion monomer into CHO-cells of the above type, either linked on the same vector on different vectors, thus creating dhfr-positive cell lines producing recombinant protein.

If the above cell lines are grown in the presence of the cytotoxic dhfr-inhibitor methotrexate, the new cell lines resistant to methotrexate will emerge. These cell lines may produce recombinant protein at an increased rate due to the amplified number of linked dhfr and the desired protein's transcriptional units. When propagating these cell lines in increasing concentrations of methotrexate (1-10,000 nM), new cell lines can be obtained which produce the desired protein at very high rate.

The above cell lines producing the desired protein can be grown on a large scale, either in suspension culture or on various solid supports. Examples of these supports are micro carriers based on dextran or collagen matrices, or solid supports in the form of hollow fibers or various ceramic materials. When grown in cell suspension culture or on micro carriers the culture of the above cell lines can be performed either as a bath culture or as a perfusion culture with continuous production of conditioned medium over extended periods of time. Thus, according to the present disclosure, the above cell lines are well suited for the development of an industrial process for the production of the desired recombinant proteins.

### Purification and Formulation

The recombinant protein can be concentrated and purified by a variety of biochemical and chromatographic methods, including methods utilizing differences in size, charge, hydrophobicity, solubility, specific affinity, etc., between the desired protein and other substances in the host cell or cell cultivation medium.

An example of such purification is the adsorption of the recombinant protein to a monoclonal antibody directed to e.g. the Fc portion of the Fc multimer or another Fc-binding ligand (e.g. protein A or protein G), which is immobilized on a solid support. After adsorption of the Fc multimer to the support, washing and desorption, the protein can be further purified by a variety of chromatographic techniques based on the above properties. The order of the purification steps is chosen, for example, according to capacity and selectivity of the steps, stability of the support or other aspects. Purification steps, for example, may be, but are not limited to, ion exchange chromatography steps, immune affinity chromatography steps, affinity chromatography steps, dye chromatography steps, and size exclusion chromatography steps.

In order to minimize the theoretical risk of virus contaminations, additional steps may be included in the process that allow effective inactivation or elimination of viruses. For example, such steps may include heat treatment in the liquid or solid state, treatment with solvents and/or detergents, radiation in the visible or UV spectrum, gamma-radiation, partitioning during the purification, or virus filtration (nano filtration).

The Fc multimers described herein can be formulated into pharmaceutical preparations for therapeutic use. The components of the pharmaceutical preparation may be resuspended or dissolved in conventional physiologically compatible aqueous buffer solutions to which there may be added, optionally, pharmaceutical excipients to provide the pharmaceutical preparation. The components of the pharmaceutical preparation may already contain all necessary pharmaceutical, physiologically compatible excipients and may be dissolved in water for injection to provide the pharmaceutical preparation.

Such pharmaceutical carriers and excipients as well as the preparation of suitable pharmaceutical formulations are well known in the art (see for example, “Pharmaceutical Formulation Development of Peptides and Proteins,” Frokjaer et al., Taylor & Francis (2000) or “Handbook of Pharmaceutical Excipients,” 3rd edition, Kibbe et al., Pharmaceutical Press (2000)). In certain embodiments, a pharmaceutical composition can comprise at least one additive such as a bulking agent, buffer, or stabilizer. Standard pharmaceutical formulation techniques are well known to persons skilled in the art (see, e.g., 2005 Physicians' Desk Reference®, Thomson Healthcare: Monvale, N J, 2004; Remington: The Science and Practice of Pharmacy, 20th ed., Gennaro et al., Eds. Lippincott Williams & Wilkins: Philadelphia, Pa., 2000). Suitable pharmaceutical additives include, e.g., sugars like mannitol, sorbitol, lactose, sucrose, trehalose, or others, amino acids like histidine, arginine, lysine, glycine, alanine, leucine, serine, threonine, glutamic acid, aspartic acid, glutamine, asparagine, phenylalanine, proline, or others, additives to achieve isotonic conditions like sodium chloride or other salts, stabilizers like Polysorbate 80, Polysorbate 20, Polyethylene glycol, propylene glycol, calcium chloride, or others, physiological pH buffering agents like Tris(hydroxymethylaminomethan), and the like. In certain embodiments, the pharmaceutical compositions may contain pH buffering reagents and wetting or emulsifying agents. In further embodiments, the compositions may contain preservatives or stabilizers. In particular, the pharmaceutical preparation comprising the Fc multimers described herein may be formulated in lyophilized or stable soluble form. The Fc multimers may be lyophilized by a variety of procedures known in the art. Lyophilized formulations are reconstituted prior to use by the addition of one or more pharmaceutically acceptable diluents such as sterile water for injection or sterile physiological saline solution or a suitable buffer solution.

The composition(s) of the pharmaceutical preparation of Fc multimer may be delivered to the individual by any pharmaceutically suitable means. Various delivery systems are known and can be used to administer the composition by any convenient route. The composition(s) of the pharmaceutical preparation of the Fc multimer can be formulated for intravenous or non-intravenous injection or for enteral (e.g., oral, vaginal, or rectal) delivery according to conventional methods. For non-intravenous administration, the composition(s) of the Fc multimer can be formulated for subcutaneous, intramuscular, intra-articular, intraperitoneal, intracerebral, intrathecal, intrapulmonary (e.g. nebulized), intranasal, intradermal, peroral or transdermal administration. In one embodiment, the composition(s) of the Fc multimer are formulated for intravenous injection. In other embodiments, the composition(s) of the Fc multimer are formulated for subcutaneous, intramuscular, or transdermal administration, preferably for subcutaneous administration. The formulations can be administered continuously by infusion or by bolus injection. Some formulations can encompass slow release systems.

The composition(s) of the pharmaceutical preparation of Fc multimer is/are administered to patients in a therapeutically effective dose. The term “therapeutically effective,” as used herein, describes a dose that is sufficient to produce the desired effects, preventing or lessening the severity or spread of an immune complex-mediated kidney disorder, or to exhibit a detectable therapeutic or preventative effect, without teaching a dose which produces intolerable adverse side effects. The exact dose depends on many factors as, for example, the formulation and mode of administration. The therapeutically effective amount can be initially estimated in cell culture assays or in animal models, for example rodent, rabbit, dog, pig, or primate models. Such information can then be used to determine useful doses and routes for administration in humans.

In one embodiment, the dose of the Fc multimer for one intravenous or one non-intravenous injection is less than 1,000 mg/kg body weight, less than 800 mg/kg body weight, less than 600 mg/kg body weight, less than 400 mg/kg body weight, less than 200 mg/kg body weight, or less than 100 mg/kg body weight. For example, in one embodiment, the dose of Fc multimer is from about 0.1 mg/kg body weight to about 1,000 mg/kg body weight, from about 1 mg/kg body weight to about 800 mg/kg body weight, from about 1 mg/kg body weight to about 700 mg/kg body weight, from about 1 mg/kg body weight to about 600 mg/kg body weight, from about 1 mg/kg body weight to about 500 mg/kg body weight, from about 1 mg/kg body weight to about 400 mg/kg body weight, from about 1 mg/kg body weight to about 300 mg/kg body weight, or from about 3 mg/kg body weight to about 200 mg/kg body weight. In one embodiment, the dose of Fc multimer is from about 3 mg/kg body weight to about 1,000 mg/kg body weight, from about 3 mg/kg body weight to about 800 mg/kg body weight, from about 3 mg/kg body weight to about 600 mg/kg body weight, from about 3 mg/kg body weight to about 500 mg/kg body weight, from about 3 mg/kg body weight to about 400 mg/kg body weight, from about 3 mg/kg body weight to about 300 mg/kg body weight, from about 3 mg/kg body weight to about 200 mg/kg body weight, or from about 3 mg/kg body weight to about 100 mg/kg body weight. In one embodiment the dose of Fc multimer is from about 10 mg/kg body weight to about 500 mg/kg body weight, from about 10 mg/kg body weight to about 400 mg/kg body weight, from about 10 mg/kg body weight to about 300 mg/kg body weight, or from about 10 mg/kg body weight to about 200 mg/kg body weight.

In a separate embodiment, the pharmaceutical composition(s) of Fc multimer is administered alone or in conjunction with other therapeutic agents. In one embodiment, these agents are incorporated as part of the same pharmaceutical. In one embodiment, the Fc multimer is administered in conjunction with an immunosuppressant therapy, such as a steroid. In another embodiment, the Fc multimer is administered with any B cell or T cell modulating agent or immunomodulator.

The administration frequency of the Fc multimer depends on many factors such as the formulation, dosage, and mode of administration. In one embodiment, a dose of Fc multimer is administered multiple times every day, once every day, once every other day, once every third day, twice per week, once per week, once every two weeks, once every three weeks, or once per month.

Therapeutic Effects

The term “therapeutic effects,” as used herein, describes treating the disease or disorder by improving parameters that characterize it, or, alternatively, preventing those disease/disorder parameters altogether. For example, therapeutic effects can be determined (1) in vitro in cell culture models, or (2) in vivo in mouse models of disease by administering a dose of an Fc multimer. A dose of Fc multimer can be 10 to 1000 mg/kg, for example, 200 mg/kg. The Fc multimer can be administered by intravenous or non-intravenous injection or intravenous infusion. Clinical assessments of animals can be made at predetermined times until a final time point after administration of the Fc multimer. Clinical assessments can include scoring based on clinical manifestations of the specific disease or disorder. Biological samples can also be taken from the animals at predetermined times until a final time point after administration of the Fc multimer. The term “biological samples,” as used herein, refers to, for example, tissue, blood, and urine. The biological samples can then be assessed for improvements in markers or indicators of immune complex-mediated kidney disorder.

The term “induce,” as used herein, is defined as to cause, produce, effect, create, give rise to, lead to, or promote.

In a preferred embodiment, a therapeutic effect of the Fc multimer can be indicated by a reduction in urine albumin levels in a mouse model of anti-GBM glomerulonephritis upon treatment with an Fc multimer. A mouse model of anti-GBM glomerulonephritis is described by Otten et al. (Otten et al., J Immunol 2009; 183:3980-3988). In this study, authors concluded that both FcγR and complement processes were obligatory for a full-blown inflammation in a novel attenuated passive model of anti-GBM disease. In this model, subnephritogenic doses of rabbit anti-GBM antibodies are administered to the animal, followed by a fixed dose of mouse mAbs to rabbit IgG, allowing timing and dosing for the induction of glomerulonephritis. This resulted in a reproducible complement activation via the classical pathway of complement and albuminuria in wild type mice. Albuminuria was not detected in mice which were FcR-γ-chain^{-/-} or mice which were reduced in C3^{-/-} suggesting a role for both were FcR-γ and complement in the pathogenesis of this model. Additionally, because C1q^{-/-} and C4^{-/-} mice supposedly lacking a functional classical and lectin pathway were found developed albuminuria, Otten et al. suggested involvement of the alternative complement pathway.

Activation of the Classical Complement Pathway

The classical complement pathway mediates the specific antibody response and is mediated by a cascade of complement components. The cascade is mainly activated by antigen-antibody complexes. The initial component of the pathway is the protein complex C1, which is comprised of one C1q and two subunits of C1r2s2. Binding of an immunoglobulin to C1q effects the first step of activation of the classical complement pathway through activation of C1r2s2 into catalytically active subunits. The activated C1s cleaves C4 into C4a and C4b and C2 into C2a and C2b. C2a then binds C4b to form C4b2a, which is also known as C3 convertase. C3 convertase catalyzes the cleavage of C3 into C3a and C3b. C3b can then bind to activated C4b2a to form C4b2a3b, which is also known as C5 convertase. C5 convertase converts C5 to fragments C5a and C5b. C5b, together with the C6, C7, C8, and C9 components, forms a complex known as the C5b-9 complex. This complex is also known as the membrane attack complex (MAC) or terminal complement complex (TCC) and forms transmembrane channels in target cells, leading to cell lysis.

“Activation of the complete classical complement pathway”, as used herein, is defined as the activation of every step of the entire classical complement pathway as described above. Activation of the complete classical complement pathway can be determined by investigating binding of the Fc multimer to C1q, the first step in activation of the classical complement pathway, and formation of C4a, C5a or soluble or membrane bound C5b-9 complex, the final effector in the classical complement pathway. For example, an Fc multimer does not induce complete activation of the classical complement pathway if the protein binds C1q but soluble C5b-9 is essentially not formed, i.e. only less than 50% of the respective positive control is formed, preferably less than 40%, preferably less than 30%, preferably less than 20%, preferably less than 10%, more preferably less than 5%. Activation of the classical complement pathway can also be determined by assessing the generation of C4a, cleavage of C2, or formation of C3 convertase. For example, an Fc multimer does not induce activation of the complete classical complement pathway if it induces the generation of C4a but either does not induce cleavage of C2 or does not induce formation of C3 convertase. “Not induce” means less than 50%, preferably less than 40%, preferably less than 30%, preferably less than 20%, preferably less than 10%, more preferably less than 5% of the respective positive control is formed.

The ability of an Fc multimer to bind C1q can be determined by an in vitro binding assay, such as an enzyme-linked immunosorbent assay (ELISA). For example, wells of a 96-well plate can be pre-coated with human C1q followed by the addition of Fc multimers. Purified peroxidase-labeled anti-human IgG conjugate can be added and bound conjugate can be visualized by using a color-producing peroxidase substrate, such as 3,3′,5,5′ tetramethylbenzidine (TMB).

Activation of the classical complement pathway by an Fc multimer can be determined by in vitro assays and indicated by generation of C4a and soluble C5b-9. For example, different concentrations of an Fc multimer can be incubated in whole blood or serum for a pre-determined period of time and any resulting generation of C4a or soluble C5b-9 (sC5b-9) can be determined by immunodetection, such as ELISA. Concentrations of Fc multimer used may be 0.01 mg/ml to 2 mg/ml, for example, 0.04 mg/ml, 0.2 mg/ml, or 1.0 mg/ml.

Generation of C4a and sC5b-9 induced by an Fc multimer can be compared relative to the generation of these components induced by heat-aggregated gamma globulin (HAGG), a potent activator of the classical complement pathway. For example, this assay can be performed in whole blood. According to some embodiments, as described in WO2017/129737, the Fc multimer induces less than 50% sC5b-9 generation, less than 40% sC5b-9 generation, less than 30% sC5b-9 generation, less than 20% sC5b-9 generation, or less than 10% sC5b-9 generation as compared to sC5b-9 generation induced by HAGG. In one embodiment, the Fc multimer induces less than 20% sC5b-9 generation in whole blood as compared to sC5b-9 generation induced by HAGG in whole blood. In another embodiment, the Fc multimer induces less than 10% sC5b-9 generation in whole blood as compared to sC5b-9 generation induced by HAGG in whole blood. In yet another embodiment, the Fc multimer induces no sC5b-9 generation.

The term “normal human serum activated with heat aggregated IgG” as used herein refers to a normal human serum sample where cleavage of nearly all C4 has been induced with heat aggregated IgG.

Activation of the classical complement pathway by an Fc multimer can also be determined by detecting C2 protein. If C2 protein is cleaved to C2a and C2b, the level of C2 protein decreases, indicating activation of the classical complement pathway. Different concentrations of an Fc multimer can be incubated in whole blood or serum for a pre-determined period of time, for example 2 h, following which C2 protein levels can be determined by immunodetection, such as immunoblotting. Activation of the classical complement pathway is indicated by cleavage of the C2 protein. The level of C2 protein in normal human serum can be compared to the level of C2 protein resulting after pre-incubation with an Fc multimer to determine the amount of C2 cleavage, and therefore activation of the classical complement pathway. A known activator of the classical complement pathway, such as HAGG, can be used as a positive control for inducing cleavage of the majority of the C2 protein in normal human serum. The term “majority,” as used herein, is defined as comprising greater than 50%, greater than 60%, greater than 70%, greater than 80%, or greater than 90%. In some embodiments, as described in WO2017/129737, the Fc multimer does not induce the cleavage of the majority of C2 protein.

Activation of the classical complement pathway by an Fc multimer can also be determined by assessing formation of C3 convertase. As described above, C3 convertase consists of the C2a and C4b subunits (C4b2a). If C2 protein is not cleaved to C2a and C2b, C3 convertase cannot be formed. As such, C3 convertase formation can be assessed as described above for determining C2 protein cleavage. In some embodiments, as described in WO2017/129737, the Fc multimer does not induce formation of C3 convertase.

Inhibition of the Classical Complement Pathway

Inhibition of the classical complement pathway by an Fc multimer can be determined by determining inhibition of C5a and sC5b-9 generation or by determining inhibition of cleavage of C2 protein. Different concentrations of the Fc multimer can be incubated in whole blood or serum with a known activator of the classical complement pathway. The level of sC5b-9 generated in the presence of an Fc multimer and a known activator of the classical complement pathway can then be compared to the level of sC5b-9 generated with the known activator of the classical complement pathway alone. The level of sC5b-9 generated can be determined as described above. The concentrations of Fc multimer used may be 0.01 mg/ml to 2 mg/ml, for example, 0.04 mg/ml, 0.2 mg/ml, or 1.0 mg/ml. The known activator of the classical complement pathway may be HAGG. The lower the level of sC5b-9 generated in the presence of an Fc multimer and an activator of the classical complement pathway is in comparison to the level of sC5b-9 generated in the presence of an activator of the classical complement pathway alone, the greater is the inhibition of sC5b-9 generation by the Fc multimer. In some embodiments, the Fc multimer inhibits greater than 50% sC5b-9 generation, greater than 60% sC5b-9 generation, greater than 70% sC5b-9 generation, greater than 80% sC5b-9 generation or greater than 90% sC5b-9 generation as compared to sC5b-9 generation induced by HAGG. In one embodiment, as described in WO2017/129737, the Fc multimer inhibits greater than 80% of sC5b-9 generation induced by HAGG.

The term “inhibit,” as used herein, is defined as to suppress, restrict, prevent, interfere with, stop, or block.

Inhibition of cleavage of C2 protein can be similarly determined. Different concentrations of the Fc multimer can be incubated in whole blood or serum with a known activator of the classical complement pathway. The greater the level of C2 protein in the presence of an Fc multimer and a known activator of the classical complement pathway compared to the level of C2 protein in the presence of the known activator of the classical complement pathway alone, the greater is the inhibition of C2 cleavage by the Fc multimer. The level of C2 protein can be determined as described above. The concentrations of Fc multimer used may be 0.01 mg/ml to 2 mg/ml, for example, 0.04 mg/ml, 0.2 mg/ml, or 1.0 mg/ml. The known activator of the classical complement pathway can be HAGG. In some embodiments, as described in WO2017/129737, the Fc multimer inhibits the cleavage of the majority of C2 protein by HAGG.

Inhibition of the classical complement pathway can also be determined using a hemolysis assay for the classical complement pathway using antibody-sensitized, or opsonized, erythrocytes. For example, sheep erythrocytes, or red blood cells, can be opsonized with rabbit anti-sheep antibodies. Normal human serum (NHS) will induce lysis of opsonized erythrocytes. Fc proteins can be pre-incubated with NHS and then added to the erythrocytes and incubated for 1 h at 37° C. The concentration of Fc construct can be from 1-1000 μg/ml, for example 2.5, 25, 50, 125, 250, or 500 μg/ml. Alternatively, Fc monomer can also be pre-incubated with NHS at the same concentrations as indicated for the Fc construct. After incubation, the mixture can be centrifuged and the degree of lysis can be determined by measuring the absorbance of released hemoglobin at 412 nm of the supernatant.

Inhibition of the classical complement pathway by the Fc multimer can be indicated by reduced lysis of erythrocytes in the mixtures that contain Fc multimer compared to the mixtures that have NHS but not Fc multimer. Inhibition of lysis of opsonized red blood cells by an Fc multimer can also be compared to lysis of opsonized red blood cells in the presence of the Fc monomer. In some embodiments, the Fc multimer inhibits lysis of opsonized sheep red blood cells as compared to Fc monomer. In one embodiment, as described in WO2017/129737, the Fc multimer inhibits lysis of opsonized sheep red blood cells by over 70% as compared to Fc monomer.

In an embodiment of the present invention, the Fc multimer prevents the pathogenesis of an immune complex-mediated kidney disorder by inhibiting activation of the classical complement pathway or the alternative complement pathway.

Activation of the Antibody-Dependent Cellular Toxicity

Antibody-dependent cellular cytotoxicity, also referred to as antibody-dependent cell-medicated cytotoxicity (ADCC), is the process by which antibodies coat a target cell and recruit effector cells to induce target cell death via non-phagocytic mechanisms (Zahavi et al. Antibody Therapeutics 2018; 1(1):7-12). Antibodies bind to their specific antigens on the target cell surface via their antigen-binding fragment (Fab) portions and interact with effector cells via their fragment crystallizable region (Fc) portions thereby acting as bridges that link the effector to a target. An effector cell is able to carry out ADCC by the expression of Fc receptors (FcR) that will bind the antibody. ADCC activity has been reported for neutrophils, monocytes and Fc receptor-bearing (FcR⁺) T and non-T lymphocytes (Katz et al., J Clin Invest. 1980; 65(1):55-63). The known classes of FcR include FcγR, which bind IgG; FcαR, which bind IgA; and FcεR, which bind IgE. FcγR are the most important for tumor cell clearance by myeloid cells and are comprised of activating FcγRI (CD64), FcγRIIA (CD32A), FcγRIIIA (CD16A), and inhibitory FcγRIIB (CD32B) receptors (Zahavi et al.). Upon binding of the FcγR to the Fc portion of the antibody, a receptor cross-linking and downstream signal propagation occurs. Once these effector cells have been activated they mediate death of the antibody-coated target cell through cytotoxic granule release, Fas signaling, or elaboration of reactive oxygen species. The best characterized mechanism utilized in ADCC is the release of perforins and granzymes from effector cell granules.

Inhibition of the Antibody-Dependent Cellular Toxicity

High physiological levels of immunoglobulin G has been found to strongly inhibit ADCC in antibody-based cancer treatment aimed at inducing ADCC (Preithner et al. Mol Immunol. 2006; 43(8):1183-93). The explanation for this observation can be found in the competition of serum IgG and therapeutic antibodies for binding to Fc receptors. This competition mechanism can be exploited in the treatment of autoimmune and inflammatory diseases. As previously mentioned, one of the mechanisms of action proposed for the anti-inflammatory effect of high-dose IVIG is the blockage of Fcγ receptors (FcγRs). In some embodiments, the Fc multimers of the present invention are capable of binding to Fc receptors, competing with disease antibodies, and inhibiting antibody-dependent cellular toxicity.

### EXEMPLARY EMBODIMENTS

In some aspects, the invention is directed to an Fc multimer for use in the treatment of an immune complex-mediated kidney disorder, wherein the Fc multimer comprises two to six IgG Fc fusion monomers, wherein each Fc fusion monomer comprises two Fc fusion polypeptide chains, wherein each Fc fusion polypeptide chain comprises an IgG Fc polypeptide and a multimerization domain; and wherein the Fc multimer lacks any mutation to increase binding affinity of the Fc multimer to a complement system protein. In some aspects of the invention, the excluded mutation is at least one of S267E, H268F, S324T, N297A, T299A, P238D, E233P, G236R, L234V, E233P, L234A, L235A, P238D, D265A, D265W, N297A, N297Q, T299A, and L328F in an IgG1 Fc domain of the Fc multimer. In a preferred aspect of the invention the excluded mutations are S267E, H268F and S324T. In another preferred aspect of the invention the excluded mutations are S267E, H268F, S324T, and N297A. In another preferred aspect of the invention the excluded mutations are S267E, H268F, S324T, N297A, L234A, and L235A. In another preferred aspect of the invention the excluded mutations are S267E, H268F, S324T, N297A, E233P, L234V, L235A and a deletion of G236. In another preferred aspect of the invention the excluded mutations are S267E, H268F, S324T, L234A, L235A. In another preferred aspect of the invention the excluded mutations are S267E, H268F, S324T, E233P, L234V, L235A, and a deletion of G236. In another preferred aspect of the invention the excluded mutations are S267E, H268F, S324T, and D265A. In another preferred aspect of the invention the excluded mutations are E233P, G236R, S267E, H268F, and S324T. In another preferred aspect of the invention the excluded mutations are G236R, S267E, H268F, S324T. In another preferred aspect of the invention the excluded mutations are E233P, G236R, S267E, H268F, S324T, and L328F. In another preferred aspect of the invention the excluded mutations are P238D, D265G, S267E, H268F, and S324T. In another preferred aspect of the invention the excluded mutations are P238D, D265W, S267E, H268F, and S324T. In another preferred aspect of the invention the excluded mutations are E233P, L234V, L235A, S267E, H268F, N297A, S324T, S328F, and a deletion of G236. In another preferred aspect of the invention the excluded mutations are S267E, H268F, S324T, and L328F. In another preferred aspect of the invention the excluded mutations are P238D, S267E, H268F, and S324T. In another preferred aspect of the invention the excluded mutations are P238D, S267E, H268F, S324T and N297A.

In some aspects of the invention, the Fc multimer is not a stradomer. In some aspects of the invention the multimerization domain does not include an IgG2 hinge.

In some aspects, the invention is directed to an Fc multimer for use in the treatment of an immune complex-mediated kidney disorder, wherein the Fc multimer comprises two to six IgG Fc fusion monomers, wherein each Fc fusion monomer comprises two Fc fusion polypeptide chains, wherein each Fc fusion polypeptide chain comprises an IgG Fc polypeptide and a multimerization domain, and wherein the multimerization domain does not include an IgG2 hinge. In some aspects of the invention, the Fc multimer is not a stradomer.

In some aspects, the invention is directed to an Fc multimer for use in the treatment of an immune complex-mediated kidney disorder, wherein the Fc multimer comprises two to six IgG Fc fusion monomers, wherein each Fc fusion monomer comprises two Fc fusion polypeptide chains, wherein each Fc fusion polypeptide chain comprises an IgG Fc polypeptide and a multimerization domain; and wherein the Fc multimer is not a stradomer.

In some aspects of the invention, the Fc multimer is a hexamer comprising six IgG Fc fusion monomers.

In some aspects of the invention the Fc multimer further comprises a linker sequence between the IgG Fc polypeptide and the multimerization domain.

In some aspects of the invention the multimerization domain comprises an IgM tailpiece. In some aspects of the invention the IgM tailpiece comprises 18 or less amino acids from an IgM tailpiece domain. In some aspects of the invention the IgM tailpiece comprises residues 233 to 250 of SEQ ID NO:1. In some aspects of the invention the IgM tailpiece is fused to a 232 amino acid sequence segment of a C-terminus of the IgG Fc polypeptide.

In some aspects of the invention the IgG Fc polypeptide comprises an IgG1 Fc polypeptide. In some aspects of the invention the IgG1 Fc polypeptide comprises a CH2 domain that is truncated at the N-terminus end and/or a CH3 domain that is truncated at the C-terminus end.

In some aspects of the invention at least one Fc fusion polypeptide chain further comprises an immunoglobulin hinge region, a fragment thereof, a variation thereof, or a modified form thereof. In some aspects of the invention at least one Fc fusion polypeptide chain further comprises an IgG1 hinge region. In some aspects of the invention at least one Fc fusion polypeptide chain further comprises an immunoglobulin hinge region comprising residues 1 to 15 of SEQ ID NO:1. In some aspects of the invention at least one Fc fusion polypeptide chain comprises SEQ ID NO:1.

In some aspects of the invention at least one Fc fusion polypeptide chain comprises SEQ ID NO:2, or residues 20 to 269 of SEQ ID NO:2. In some aspects of the invention at least one Fc fusion polypeptide chain comprises SEQ ID NO:3 wherein a leucine is mutated to a cysteine at position 309 of the IgG Fc polypeptide of at least one Fc fusion polypeptide chain. In some aspects of the invention at least one Fc fusion polypeptide chain comprises SEQ ID NO:4, or residues 20 to 269 of SEQ ID NO:4, and a leucine is mutated to a cysteine at position 309 of the IgG Fc polypeptide of at least one Fc fusion polypeptide chain.

In some aspects of the invention at least one Fc fusion polypeptide chain has up to 5 conservative amino acid changes.

In some aspects of the invention at least one Fc fusion polypeptide chain does not comprise a Fab polypeptide.

In some aspects, the invention is directed to a recombinant human Fc hexamer for use in the treatment of an immune complex-mediated kidney disorder, wherein the recombinant human Fc hexamer comprises six human IgG1 Fc fusion monomers, wherein each Fc fusion monomer comprises two human Fc fusion polypeptide chains and each Fc fusion polypeptide chain comprises a human IgG1 Fc polypeptide and a human IgM tailpiece, and further wherein the IgM tailpiece in each Fc fusion polypeptide chain comprises 18 amino acids fused with 232 amino acids at a C-terminus of a constant region of the IgG1 Fc polypeptide.

In some aspects of the invention the Fc hexamer lacks any mutation to increase binding affinity of the Fc hexamer to a complement system protein. In some aspects of the invention the excluded mutation is a least one of S267E, H268F, S324T, N297A, T299A, P238D, E233P, G236R, L234V, E233P, L234A, L235A, P238D, D265A, D265W, N297A, N297Q, T299A, and L328F in an IgG1 Fc domain of the Fc hexamer. In a preferred aspect of the invention the excluded mutations are S267E, H268F and S324T. In another preferred aspect of the invention the excluded mutations are S267E, H268F, S324T, and N297A. In another preferred aspect of the invention the excluded mutations are S267E, H268F, S324T, N297A, L234A, and L235A. In another preferred aspect of the invention the excluded mutations are S267E, H268F, S324T, N297A, E233P, L234V, L235A and a deletion of G236. In another preferred aspect of the invention the excluded mutations are S267E, H268F, S324T, L234A, L235A. In another preferred aspect of the invention the excluded mutations are S267E, H268F, S324T, E233P, L234V, L235A, and a deletion of G236. In another preferred aspect of the invention the excluded mutations are S267E, H268F, S324T, and D265A. In another preferred aspect of the invention the excluded mutations are E233P, G236R, S267E, H268F, and S324T. In another preferred aspect of the invention the excluded mutations are G236R, S267E, H268F, S324T. In another preferred aspect of the invention the excluded mutations are E233P, G236R, S267E, H268F, S324T, and L328F. In another preferred aspect of the invention the excluded mutations are P238D, D265G, S267E, H268F, and S324T. In another preferred aspect of the invention the excluded mutations are P238D, D265W, S267E, H268F, and S324T. In another preferred aspect of the invention the excluded mutations are E233P, L234V, L235A, S267E, H268F, N297A, S324T, S328F, and a deletion of G236. In another preferred aspect of the invention the excluded mutations are S267E, H268F, S324T, and L328F. In another preferred aspect of the invention the excluded mutations are P238D, S267E, H268F, and S324T. In another preferred aspect of the invention the excluded mutations are P238D, S267E, H268F, S324T and N297A.

In some aspects of the invention the Fc hexamer is not a stradomer.

In some aspects of the invention each Fc fusion polypeptide chain of the Fc hexamer further comprises an IgG1 hinge region and does not comprise a Fab polypeptide.

In some aspects of the invention, each IgG1 Fc polypeptide of the Fc hexamer comprises a leucine to cysteine mutation at position 309.

In some aspects, the invention is directed to an Fc multimer for use in the treatment of an immune complex-mediated kidney disorder, wherein the Fc multimer comprises four polypeptides that form three Fc monomers, wherein the first polypeptide comprises a first Fc polypeptide, a first linker, and a second Fc polypeptide, wherein the second polypeptide comprises a third Fc polypeptide, a second linker, and a fourth Fc polypeptide, wherein the third polypeptide comprises a fifth Fc polypeptide, wherein the fourth polypeptide comprises a sixth Fc polypeptide, wherein the first Fc polypeptide and the third Fc polypeptide form the first Fc monomer, wherein the fifth Fc polypeptide and the second Fc polypeptide form the second Fc monomer, and wherein the sixth Fc polypeptide and fourth Fc polypeptide form the third Fc monomer.

In some aspects of the invention the Fc multimer lacks any mutation to increase binding affinity of the Fc multimer to a complement system protein. In some aspects of the invention the excluded mutation is at least one of S267E, H268F, S324T, N297A, T299A, P238D, E233P, G236R, L234V, E233P, L234A, L235A, P238D, D265A, D265W, N297A, N297Q, T299A, and L328F in an IgG1 Fc domain of the Fc multimer. In a preferred aspect of the invention the excluded mutations are S267E, H268F and S324T. In another preferred aspect of the invention the excluded mutations are S267E, H268F, S324T, and N297A. In another preferred aspect of the invention the excluded mutations are S267E, H268F, S324T, N297A, L234A, and L235A. In another preferred aspect of the invention the excluded mutations are S267E, H268F, S324T, N297A, E233P, L234V, L235A and a deletion of G236. In another preferred aspect of the invention the excluded mutations are S267E, H268F, S324T, L234A, L235A. In another preferred aspect of the invention the excluded mutations are S267E, H268F, S324T, E233P, L234V, L235A, and a deletion of G236. In another preferred aspect of the invention the excluded mutations are S267E, H268F, S324T, and D265A. In another preferred aspect of the invention the excluded mutations are E233P, G236R, S267E, H268F, and S324T. In another preferred aspect of the invention the excluded mutations are G236R, S267E, H268F, S324T. In another preferred aspect of the invention the excluded mutations are E233P, G236R, S267E, H268F, S324T, and L328F. In another preferred aspect of the invention the excluded mutations are P238D, D265G, S267E, H268F, and S324T. In another preferred aspect of the invention the excluded mutations are P238D, D265W, S267E, H268F, and S324T. In another preferred aspect of the invention the excluded mutations are E233P, L234V, L235A, S267E, H268F, N297A, S324T, S328F, and a deletion of G236. In another preferred aspect of the invention the excluded mutations are S267E, H268F, S324T, and L328F. In another preferred aspect of the invention the excluded mutations are P238D, S267E, H268F, and S324T. In another preferred aspect of the invention the excluded mutations are P238D, S267E, H268F, S324T and N297A.

In some aspects, the invention is directed to a method of treating an immune complex-mediated kidney disorder comprising administering an Fc multimer to a subject, wherein the Fc multimer comprises two to six IgG Fc fusion monomers, wherein each Fc fusion monomer comprises two Fc fusion polypeptide chains, wherein each Fc fusion polypeptide chain comprises an IgG Fc polypeptide and a multimerization domain, and wherein the Fc multimer lacks any mutation to increase binding affinity of the Fc multimer to a complement system. In some aspects of the invention the excluded mutation is at least one of S267E, H268F, S324T, N297A, T299A, P238D, E233P, G236R, L234V, E233P, L234A, L235A, P238D, D265A, D265W, N297A, N297Q, T299A, and L328F in an IgG1 Fc domain of the Fc multimer. In a preferred aspect of the invention the excluded mutations are S267E, H268F and S324T. In another preferred aspect of the invention the excluded mutations are S267E, H268F, S324T, and N297A. In another preferred aspect of the invention the excluded mutations are S267E, H268F, S324T, N297A, L234A, and L235A. In another preferred aspect of the invention the excluded mutations are S267E, H268F, S324T, N297A, E233P, L234V, L235A and a deletion of G236. In another preferred aspect of the invention the excluded mutations are S267E, H268F, S324T, L234A, L235A. In another preferred aspect of the invention the excluded mutations are S267E, H268F, S324T, E233P, L234V, L235A, and a deletion of G236. In another preferred aspect of the invention the excluded mutations are S267E, H268F, S324T, and D265A. In another preferred aspect of the invention the excluded mutations are E233P, G236R, S267E, H268F, and S324T. In another preferred aspect of the invention the excluded mutations are G236R, S267E, H268F, S324T. In another preferred aspect of the invention the excluded mutations are E233P, G236R, S267E, H268F, S324T, and L328F. In another preferred aspect of the invention the excluded mutations are P238D, D265G, S267E, H268F, and S324T. In another preferred aspect of the invention the excluded mutations are P238D, D265W, S267E, H268F, and S324T. In another preferred aspect of the invention the excluded mutations are E233P, L234V, L235A, S267E, H268F, N297A, S324T, S328F, and a deletion of G236. In another preferred aspect of the invention the excluded mutations are S267E, H268F, S324T, and L328F. In another preferred aspect of the invention the excluded mutations are P238D, S267E, H268F, and S324T. In another preferred aspect of the invention the excluded mutations are P238D, S267E, H268F, S324T and N297A.

In some aspects of the invention the Fc multimer is not a stradomer. In some aspects of the invention the multimerization domain does not include an IgG2 hinge.

In some aspects, the invention is directed to a method of treating an immune complex-mediated kidney disorder comprising administering an Fc multimer to a subject, wherein the Fc multimer comprises two to six IgG Fc fusion monomers, wherein each Fc fusion monomer comprises two Fc fusion polypeptide chains, wherein each Fc fusion polypeptide chain comprises an IgG Fc polypeptide and a multimerization domain, and wherein the multimerization domain does not include an IgG2 hinge. In some aspects of the invention the Fc multimer is not a stradomer.

In some aspects, the invention is directed to a method of treating an immune complex-mediated kidney disorder comprising administering an Fc multimer to a subject, wherein the Fc multimer comprises two to six IgG Fc fusion monomers, wherein each Fc fusion monomer comprises two Fc fusion polypeptide chains, wherein each Fc fusion polypeptide chain comprises an IgG Fc polypeptide and a multimerization domain; and wherein the Fc multimer is not a stradomer.

In some aspects of the invention the multimerization domain of the Fc multimer comprises an IgM tailpiece. In some aspects of the invention the IgM tailpiece comprises residues 233 to 250 of SEQ ID NO:1.

In some aspects of the invention the at least one Fc fusion polypeptide chain of the Fc multimer further comprises an IgG hinge region and does not comprise a Fab polypeptide. In some aspects of the invention the at least one Fc fusion polypeptide chain of the Fc multimer comprises an IgG1 hinge region and an IgG1 Fc polypeptide.

In some aspects of the invention the at least one Fc fusion polypeptide chain of the Fc multimer comprises SEQ ID NO:1 or SEQ ID NO:3. In some aspects of the invention the least one Fc fusion polypeptide chain of the Fc multimer comprises SEQ ID NO:3 and a leucine is mutated to a cysteine at position 309 of the IgG Fc polypeptide of at least one Fc fusion polypeptide chain. In some aspects of the invention at least one Fc fusion polypeptide chain comprises SEQ ID NO:4 and a leucine is mutated to a cysteine at position 309 of the IgG Fc polypeptide of at least one Fc fusion polypeptide chain.

In some aspects of the invention at least one Fc fusion polypeptide chain of the Fc multimer has up to 5 conservative amino acid changes.

In some aspects of the invention the Fc multimer is a hexamer comprising six IgG Fc fusion monomers.

In some aspects, the invention is directed to a method of treating an immune complex-mediated kidney disorder, comprising administering a recombinant human Fc hexamer to a subject, wherein the recombinant human Fc hexamer comprises six human IgG1 Fc fusion monomers, wherein each Fc fusion monomer comprises two human Fc fusion polypeptide chains and each Fc fusion polypeptide chain comprises a human IgG1 Fc polypeptide and a human IgM tailpiece, and further wherein the IgM tailpiece in each Fc fusion polypeptide chain comprises 18 amino acids fused with 232 amino acids at a C-terminus of a constant region of the IgG1 Fc polypeptide.

In some aspects of the invention, the recombinant human Fc hexamer lacks any mutation to increase binding affinity of the recombinant human Fc hexamer to a complement system protein. In some aspects of the invention the excluded mutation is at least one of S267E, H268F, S324T, N297A, T299A, P238D, E233P, G236R, L234V, E233P, L234A, L235A, P238D, D265A, D265W, N297A, N297Q, T299A, and L328F in an IgG1 Fc domain of the Fc hexamer. In a preferred aspect of the invention the excluded mutations are S267E, H268F and S324T. In another preferred aspect of the invention the excluded mutations are S267E, H268F, S324T, and N297A. In another preferred aspect of the invention the excluded mutations are S267E, H268F, S324T, N297A, L234A, and L235A. In another preferred aspect of the invention the excluded mutations are S267E, H268F, S324T, N297A, E233P, L234V, L235A and a deletion of G236. In another preferred aspect of the invention the excluded mutations are S267E, H268F, S324T, L234A, L235A. In another preferred aspect of the invention the excluded mutations are S267E, H268F, S324T, E233P, L234V, L235A, and a deletion of G236. In another preferred aspect of the invention the excluded mutations are S267E, H268F, S324T, and D265A. In another preferred aspect of the invention the excluded mutations are E233P, G236R, S267E, H268F, and S324T. In another preferred aspect of the invention the excluded mutations are G236R, S267E, H268F, S324T. In another preferred aspect of the invention the excluded mutations are E233P, G236R, S267E, H268F, S324T, and L328F. In another preferred aspect of the invention the excluded mutations are P238D, D265G, S267E, H268F, and S324T. In another preferred aspect of the invention the excluded mutations are P238D, D265W, S267E, H268F, and S324T. In another preferred aspect of the invention the excluded mutations are E233P, L234V, L235A, S267E, H268F, N297A, S324T, S328F, and a deletion of G236. In another preferred aspect of the invention the excluded mutations are S267E, H268F, S324T, and L328F. In another preferred aspect of the invention the excluded mutations are P238D, S267E, H268F, and S324T. In another preferred aspect of the invention the excluded mutations are P238D, S267E, H268F, S324T and N297A.

In some aspects of the invention the Fc hexamer is not a stradomer.

In some aspects of the invention each Fc fusion polypeptide chain of the Fc hexamer further comprises an IgG1 hinge region and does not comprise a Fab polypeptide.

In some aspects of the invention each IgG1 Fc polypeptide of the fc hexamer comprises a leucine to cysteine mutation at position 309.

In some aspects, the invention is directed to a method of treating an immune complex-mediated kidney disorder, comprising administering an Fc multimer to a subject, wherein the Fc multimer comprises four polypeptides that form three Fc monomers, wherein the first polypeptide comprises a first Fc polypeptide, a first linker, and a second Fc polypeptide, wherein the second polypeptide comprises a third Fc polypeptide, a second linker, and a fourth Fc polypeptide, wherein the third polypeptide comprises a fifth Fc polypeptide, wherein the fourth polypeptide comprises a sixth Fc polypeptide, wherein the first Fc polypeptide and the third Fc polypeptide form together the first Fc monomer, wherein the fifth Fc polypeptide and the second Fc polypeptide form the second Fc monomer, and wherein the sixth Fc polypeptide and fourth Fc polypeptide form the third Fc monomer.

In some aspects the Fc multimer lacks any mutation to increase binding affinity of the Fc multimer to a complement system protein. In some aspects of the invention the excluded mutation is at least one of S267E, H268F, S324T, N297A, T299A, P238D, E233P, G236R, L234V, E233P, L234A, L235A, P238D, D265A, D265W, N297A, N297Q, T299A, and L328F in an IgG1 Fc domain of the Fc multimer. In a preferred aspect of the invention the excluded mutations are S267E, H268F and S324T. In another preferred aspect of the invention the excluded mutations are S267E, H268F, S324T, and N297A. In another preferred aspect of the invention the excluded mutations are S267E, H268F, S324T, N297A, L234A, and L235A. In another preferred aspect of the invention the excluded mutations are S267E, H268F, S324T, N297A, E233P, L234V, L235A and a deletion of G236. In another preferred aspect of the invention the excluded mutations are S267E, H268F, S324T, L234A, L235A. In another preferred aspect of the invention the excluded mutations are S267E, H268F, S324T, E233P, L234V, L235A, and a deletion of G236. In another preferred aspect of the invention the excluded mutations are S267E, H268F, S324T, and D265A. In another preferred aspect of the invention the excluded mutations are E233P, G236R, S267E, H268F, and S324T. In another preferred aspect of the invention the excluded mutations are G236R, S267E, H268F, S324T. In another preferred aspect of the invention the excluded mutations are E233P, G236R, S267E, H268F, S324T, and L328F. In another preferred aspect of the invention the excluded mutations are P238D, D265G, S267E, H268F, and S324T. In another preferred aspect of the invention the excluded mutations are P238D, D265W, S267E, H268F, and S324T. In another preferred aspect of the invention the excluded mutations are E233P, L234V, L235A, S267E, H268F, N297A, S324T, S328F, and a deletion of G236. In another preferred aspect of the invention the excluded mutations are S267E, H268F, S324T, and L328F. In another preferred aspect of the invention the excluded mutations are P238D, S267E, H268F, and S324T. In another preferred aspect of the invention the excluded mutations are P238D, S267E, H268F, S324T and N297A.

In some aspects of the invention the Fc multimer or the recombinant human Fc hexamer inhibits complement-dependent cytotoxicity and antibody-dependent cellular cytotoxicity in vitro.

In some aspects of the invention the Fc multimer or the recombinant human Fc hexamer inhibits activation of the classical complement pathway or the alternative complement pathway.

In some aspects of the invention the Fc multimer or the recombinant human Fc hexamer inhibits pathology in vivo in a mouse model of an immune complex-mediated kidney disorder.

In some aspects of the invention at least one Fc monomer or Fc fusion monomer of the Fc multimer or recombinant human Fc hexamer is capable of binding to an Fcγ receptor. In some aspects of the invention a first Fc monomer or Fc fusion monomer is capable of binding to a first Fcγ receptor and a second Fc monomer or Fc fusion monomer is capable of binding to a second Fcγ receptor.

In some aspects of the invention the Fc multimer or the recombinant human Fc hexamer is used in the treatment of an immune complex-mediated kidney disorder, wherein the immune complex-mediated kidney disorder is one of nephritis, glomerulonephritis, interstitial nephritis, anti-glomerular basement membrane (anti-GBM) disease, Goodpasture syndrome, autoimmune kidney disease, lupus nephritis, membranous nephropathy, membranoproliferative glomerulonephritis (MPGN), or Bright's disease. In some aspects of the invention the immune complex-mediated kidney disorder is lupus nephritis.

In some aspects of the invention the Fc multimer or recombinant human Fc hexamer is administered intravenously, subcutaneously, orally, intrathecally, or intrapulmonarily by nebulization.

In some aspects of the invention the Fc multimer or recombinant human Fc hexamer is administered to the subject in an amount ranging from about 3 mg/kg to about 200 mg/kg. In some aspects of the invention the Fc multimer or recombinant human Fc hexamer is administered in an amount ranging from about 25 mg/kg to about 500 mg/kg.

### EXAMPLES

### Example 1: Preparation of IgG1 Fc Multimers

Fc-μTP was generated by fusing the 18 amino acid residues (PTLYNVSLVMSDTAGTCY SEQ ID NO: 9) of human IgM tail piece to the C-terminus of the constant region of human IgG1 Fc fragment (amino acid residues 216-447, EU numbering; UniProtKB—P01857). Fc-μTP-L309C was generated by mutating the Leu residue at 309 (EU numbering) of Fc-μTP to Cys. The DNA fragments encoding Fc-μTP and Fc-μTP-L309C were synthesized and codon-optimized for human cell expression by ThermoFisher Scientific (MA, USA). The DNA fragments were cloned into ApaLI and XbaI sites of pRhG4 mammalian cell expression vector using InTag positive selection method (Chen, C G et al, (2014). Nucleic Acids Res 42(4):e26; Jostock T, et al (2004). J. Immunol. Methods. 289:65-80). Briefly, Fc-μTP and Fc-μTP-L309C fragments were isolated by ApaLI and AscI digestion. A CmR InTag adaptor comprising of BGH polyA addition sites (BGHpA) and chloramphenicol resistance gene (CmR) was also isolated by AscI and SpeI digestion (Chen, C G et al, (2014). Nucleic Acids Res 42(4):e26). The Fc molecules and the CmR InTag adaptor were co-cloned into ApaLI and XbaI sites of pRhG4 vector using T4 DNA ligase. Positive clones were selected on agar plates containing 34 μg/ml chloramphenicol. Miniprep plasmid DNA was purified using the QIAprep Spin Miniprep kit (QIAGEN, Hilden, Germany) and sequence confirmed by DNA sequencing analysis. The restriction enzymes and T4 DNA ligases were purchased from New England BioLabs (MA, USA).

The transient transfection using Expi293™ Expression System (Life Technologies, NY, USA) was performed according to the manufacturer's instruction. Briefly, plasmid DNA (0.8 μg) was diluted in 0.4 ml Opti-MEM and mixed gently. Expifectamine 293 Reagent (21.6 μL) was diluted in 0.4 ml Opti-MEM, mixed gently and incubated for 5 min at room temperature. The diluted Expifectamine was then added to the diluted DNA, mixed gently and incubated at room temperature for 20-30 min to allow the DNA-Expifectamine complexes to form. The DNA-Expifectamine complex was then added to the 50 ml Bioreactor tube containing 6.8 ml of Expi293 cells (2×10⁷ cells). The cells were incubated in a 37° C. incubator with 8% CO₂ shaking at 250 rpm for approximately 16-18 h. A master mix consisting of 40 μl Enhancer 1 (Life Technologies, NY, USA), 400 μl Enhancer 2 (Life Technologies, NY, USA) and 200 μl of LucraTone™ Lupin was prepared and added to each Bioreactor tube. The cells were incubated for further 4 days in a 37° C. incubator with 8% CO₂ shaking at 250 rpm. Protein was harvested from supernatant centrifugation at 4000 rpm for 20 min and filtered into a clean tube using a 0.22 μm filter before HPLC quantitation and purification.

In order to produce IgG1 Fc multimers, the C-terminus of recombinant human IgG1 Fc was fused to the 18 amino acid tailpiece of IgM. The IgM tailpiece (μTP) promotes formation of pentamers and hexamers. The Fc fusion proteins were produced with either wild-type (WT) human IgG1 Fc peptide (Fc-μTP) or a variant thereof with a point mutation of leucine to cysteine at residue 309 (Fc-μTP-L309C). The leucine 309 to cysteine point mutation (Fc-μTP-L309C) was expected to provide a more stable structure than the WT (Fc-μTP) due to the formation of covalent bonds between Fc molecules. This stabilization is accomplished without a J-chain.

The Fc-μTP and Fc-μTP-L309C fusion monomeric subunits result from two peptides comprising the following regions (residue numbers refer to those in SEQ ID NOs:2 and 4, respectively):

| | |
|---|---|
| Signal peptide | residues 1-19 |
| Hinge region of human IgG1 | residues 20-34 |
| Fc region of human IgG1 | residues 35-251 |
| Tailpiece of human IgM | residues 252-269 |

The amino acid sequences for the mature forms of the Fc-μTP and Fc-μTP-L309C peptides are provided as SEQ ID NO:1 and SEQ ID NO:3, respectively. The nucleic acid coding sequences are provided as SEQ ID NO:95 (corresponding to SEQ ID NO:9 of WO2017/129737) and SEQ ID NO:96 (corresponding to SEQ ID NO:10 of WO2017/129737), respectively.

During expression, the signal peptide is cleaved off to form the mature Fc-μTP and Fc-μTP-L309C fusion peptides. The sequences of the immature fusion peptides are provided in SEQ ID NOs:2 and 4, respectively.

SDS-PAGE of the multimeric Fc proteins showed a laddering pattern for each preparation, corresponding to monomer, dimer, trimer, tetramer, pentamer and hexamers of the Fc construct. Fc-μTP-L309C, but not Fc-μTP, had a predominant band at the expected hexamer position, which was consistent with a more stable structure under the disruptive electrophoresis buffer conditions. Higher order structures, most likely dimers of hexamer, were also evident for Fc-μTP-L309C. For the following examples, the hexamer fraction of this material was purified.

Recombinant human IgG1 Fc monomer (residues 1 to 232 of SEQ ID NO:1) was also produced and used as a control.

For construction of the trivalent Fc constructs used in Example 4, Fc DNA sequences were derived from human IgG1 Fc. Protuberance, cavity and charges mutations were substituted in the parental Fc sequence. DNA encoding a leader peptide derived from the human immunoglobulin Kappa Light chain was attached to the 5′ region. It will be understood that any one of a variety of leader peptides may be used in connection with the present invention. The leader peptide is usually clipped off in the ER lumen. An 11 nucleotide sequence containing a 5′ terminal EcoR1 site was added upstream of the ATG start codon. A 30 nucleotide sequence containing a 3′ terminal Xho1 site was added downstream of the 3′ terminal TGA translation termination codon. The DNA sequences were optimized for expression in mammalian cells and cloned into the pcDNA3.4 mammalian expression vector.

The amino acid sequences of secreted polypeptides used in Example 4 are provided below.

SIF1
SEQ ID NO: 107 or 108
DKTHTCPPCPAPELLGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKAKGQPREP
QVCTLPPSRDELTKNQVSLSCAVKGFYPSDIAVEWESNGQPENNYKTTPPVLDSDGSFFLVSK
LTVDKSRWQQGNVFSCSVMHEALHNHYTQKSLSLSPG(K)
SEQ ID NO: 113 or 114
DKTHTCPPCPAPELLGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKAKGQPREP
QVYTLPPCRDELTKNQVSLWCLVKGFYPSDIAVEWESNGQPENNYKTTPPVLDSDGSFFLYSK
LTVDKSRWQQGNVFSCSVMHEALHNHYTQKSLSLSPGKSGGGSGGGSGGGSGGGSGGGDK
THTCPPCPAPELLGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVLKSDGSFFLYSDLTV
DKSRWQQGNVFSCSVMHEALHNHYTQKSLSLSPG(K)
CC
SEQ ID NO: 124
DKTHTCPPCPAPELLGGPSVFLFPPKPKDTLMISRTPEVTCVVVDVSHEDPEVKFNWYVDGVE
VHNAKTKPPEEQYNSTYRVVSVLTVLHQDWLNGKEYKCKVSNKALPAPIEKTISKAKGQPREP
QVCTLPPSRDELTKNQVSLSCAVDGFYPSDIAVEWESNGQPENNYKTTPPVLDSDGSFFLVSK
LTVDKSRWQQGNVFSCSVMHEALHNHYTQKSLSLSPG
SEQ ID NO: 125
DKTHTCPPCPAPELLGGPSVFLFPPKPKDTLMISRTPEVTCVVVDVSHEDPEVKFNWYVDGVE
VHNAKTKPPEEQYNSTYRVVSVLTVLHQDWLNGKEYKCKVSNKALPAPIEKTISKAKGQPREP
QVYTLPPCRDKLTKNQVSLWCLVKGFYPSDIAVEWESNGQPENNYKTTPPVLDSDGSFFLYSK
LTVDKSRWQQGNVFSCSVMHEALHNHYTQKSLSLSPGKGGGGGGGGGGGGGGGGGGGGD
KTHTCPPCPAPELLGGPSVFLFPPKPKDTLMISRTPEVTCVVVDVSHEDPEVKFNWYVDGVEV
HNAKTKPPEEQYNSTYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPVLKSDGSFFLYSDLT
VDKSRWQQGNVFSCSVMHEALHNHYTQKSLSLSPG
Q1
SEQ ID NO: 122
DKTHTCPPCPAPELLGGPSVFLFPPKPKDTLMASRTPEVTCVVVDVSHEDPEVKFNWYVDGVE
VHNAKTKPPEEQYNSTYRVVSVLTVLHQDWLNGKEYKCKVSNKALPAPIEKTISKAKGQPREP
QVCTLPPSRDELTKNQVSLSCAVDGFYPSDIAVEWESNGQPENNYKTTPPVLDSDGSFFLVSK
LTVDKSRWQQGNVFSCSVMHEALHNHYTQKSLSLSPG
SEQ ID NO: 126
DKTHTCPPCPAPELLGGPSVFLFPPKPKDTLMASRTPEVTCVVVDVSHEDPEVKFNWYVDGVE
VHNAKTKPPEEQYNSTYRVVSVLTVLHQDWLNGKEYKCKVSNKALPAPIEKTISKAKGQPREP
QVYTLPPCRDKLTKNQVSLWCLVKGFYPSDIAVEWESNGQPENNYKTTPPVLDSDGSFFLYSK
LTVDKSRWQQGNVFSCSVMHEALHNHYTQKSLSLSPGKGGGGGGGGGGGGGGGGGGGGD
KTHTCPPCPAPELLGGPSVFLFPPKPKDTLMISRTPEVTCVVVDVSHEDPEVKFNWYVDGVEV
HNAKTKPPEEQYNSTYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPVLKSDGSFFLYSDLT
VDKSRWQQGNVFSCSVMHEALHNHYTQKSLSLSPG

For protein expression of the Fc constructs, two of the DNA plasmid constructs expressing the polypeptides pairs shown above were transfected into EXPI293 cells (LifeTechnologies). Liposome transfection was used to introduce plasmid DNA into EXPI293 cells. The total amount of transfected plasmid constructs was fixed whereas the ratio of different plasmid constructs was varied to maximize the yield of desired constructs.

After protein expression, the expressed constructs were purified from the cell culture supernatant by Protein A-based affinity column chromatography. Media supernatants were loaded onto a Poros MabCapture A (LifeTechnologies) column using an AKTA Avant preparative chromatography system (GE Healthcare Life Sciences). Captured Fc constructs were then washed with phosphate buffered saline (low-salt wash) followed by phosphate buffered saline supplemented with 500 mM NaCl (high-salt wash). Fc constructs were eluted with 100 mM glycine, 150 mM NaCl, pH 3 buffer. The protein solution emerging from the column was neutralized by addition of 1M TRIS pH 7.4 to a final concentration of 100 mM. The Fc constructs were further fractionated by ion exchange chromatography using Poros® XS resin (Applied Biosciences Cat. #4404336). The column was pre-equilibrated with 10 mM MES, pH 6 (buffer A), and the sample was eluted with a gradient against 10 mM MES, 500 mM sodium chloride, pH 6 (buffer B).

Purified Fc constructs were analyzed by SDS-PAGE (sodium dodecyl sulfate polyacrylamide gel electrophoresis) under both reducing and non-reducing conditions followed by Coomassie Blue staining to confirm the presence of protein bands of expected size.

### Example 2: Fc-μTP-L309C (CHO) Inhibits Anti-GBM Glomerulonephritis

The effect of Fc-μTP-L309C generated from CHO cells [Fc-μTP-L309C (CHO)] was determined in an in vivo model of anti-glomerular basement membrane (GBM) glomerulonephritis.

Briefly, anti-GBM glomerulonephritis was induced in C57BL/6 mice by intravenously (i.v., tail vein) injecting 1 mg of polyclonal rabbit anti-GBM antibody at day 0, followed by intraperitoneal (i.p.) injection on day 6 with 2 mg of the mouse monoclonal anti-rabbit IgG (MsaRb IgG produced from hybridoma CRL-1753 (ATCC)). Mice were i.p. injected with PBS or Fc-μTP-L309C at 50, 100 or 200 mg/kg on day 6, which was approximately one hour before the injection with the MsaRb IgG mAb. After the injection of MsaRb, mice were placed individually in metabolic cages (Tecniplast) to collect urine over a period of 24 hours. Urine albumin levels were measured with an ELISA kit (Bethyl Laboratories) and albuminuria per mouse is plotted as μg/24 hr.

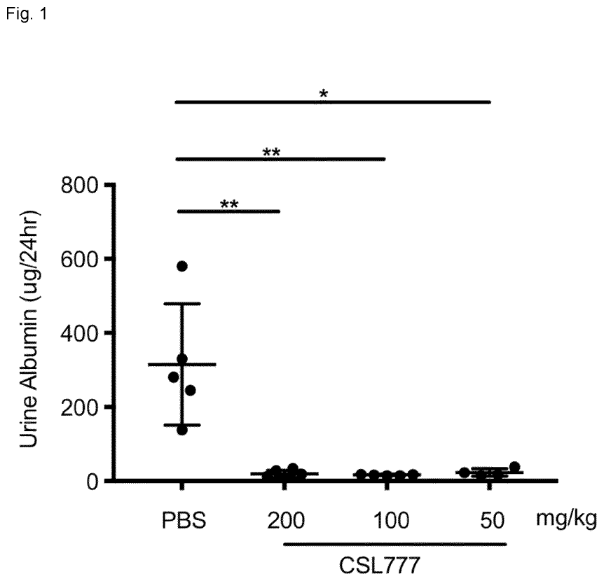

As shown in FIG. 1, urine albumin levels were significantly reduced in mice treated with Fc-μTP-L309C at all of the doses tested (PBS vs Fc-μTP-L309C at 200 mg/kg: p=0.0016; PBS vs Fc-μTP-L309C at 100 mg/kg: p=0.0036; PBS vs Fc-μTP-L309C at 50 mg/kg: p=0.0101). Table 1 shows the measured values.

TABLE 1

| Compound | Urine Albumin (ug/24 hr)* |
|---|---|
| PBS | 314.7 ± 73.5 |
| Fc-μTP-L309C at 200 mg/kg | 18.82 ± 4.03 |
| Fc-μTP-L309C at 100 mg/kg | 16.05 ± 0.73 |
| Fc-μTP-L309C at 50 mg/kg | 23.02 ± 5.17 |

*Mean ± SEM; student test

### Example 3: Fc-μTP-L309C and its Variant with Reduced C1q Binding Capacity Inhibit Anti-GBM Glomerulonephritis

The effect of Fc-μTP-L309C generated from CHO [Fc-μTP-L309C (CHO)] and HEK293 cells [Fc-μTP-L309C (HEK)] and the mutant with reduced C1q binding capacity (K322A which was generated from HEK293 cells) was determined in an in vivo model of anti-GBM glomerulonephritis.

Briefly, anti-GBM glomerulonephritis was induced in C57BL/6 mice by intravenously (i.v., tail vein) injecting 1 mg of polyclonal rabbit anti-GBM antibody at day 0, followed by intraperitoneal (i.p.) injection on day 6 with 2 mg of the mouse monoclonal anti-rabbit IgG (MsaRb IgG produced from hybridoma CRL-1753 (ATCC)). Approximately one hour before the injection with the MsaRb IgG mAb, mice were i.p. injected with PBS, Fc-μTP-L309C (CHO), Fc-μTP-L309C (HEK) or K322A at 50 mg/kg. After the injection of MsaRb, mice were placed individually in metabolic cages (Tecniplast) to collect urine over a period of 24 hours. Urine albumin levels were measured with an ELISA kit (Bethyl Laboratories) and albuminuria per mouse is plotted as μg/24 hr.

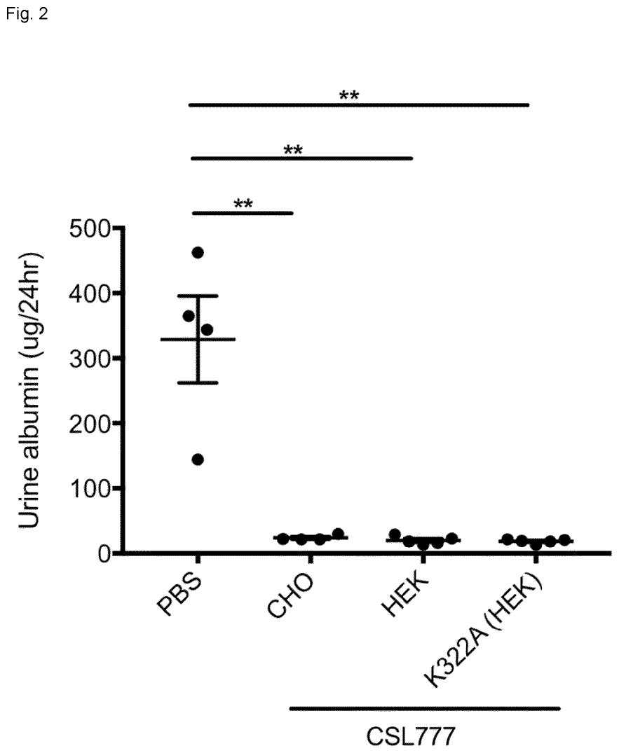

As shown in FIG. 2, urine albumin levels were significantly reduced in mice treated with all forms of Fc-μTP-L309C (PBS vs Fc-μTP-L309C generated from CHO cells: p=0.0038; PBS vs Fc-μTP-L309C from HEK293 cells: p=0.0012; PBS vs Fc-μTP-L309C K322A variant: p=0.0011). Table 2 shows the measured values.

TABLE 2

| Compound | Urine Albumin (ug/24 hr)* |
|---|---|
| PBS | 328.8 ± 66.68 |
| Fc-μTP-L309C from CHO cells | 23.88 ± 2.01 |
| Fc-μTP-L309C from HEK cells | 19.97 ± 2.79 |
| Fc-μTP-L309C K322A from HEK cells | 18.63 ± 1.49 |

*Mean ± SEM; student test

### Example 4: Fc-μTP-L309C, CC SIF1, and Q1 Inhibit Anti-GBM Glomerulonephritis

The effect of Fc-μTP-L309C generated from HEK293 cells i.e. Fc-μTP-L309C (HEK), and trivalent Fc multimers (as described in Example 1) CC, SIF1 and Q1 was determined in an in vivo model of anti-GBM glomerulonephritis.

Briefly, anti-GBM glomerulonephritis was induced in C57BL/6 mice by intravenously (i.v., tail vein) injecting 1 mg of polyclonal rabbit anti-GBM antibody at day 0, followed by intraperitoneal (i.p.) injection on day 6 with 2 mg of the mouse monoclonal anti-rabbit IgG (MsaRb IgG produced from hybridoma CRL-1753 (ATCC)). Approximately one hour before the injection with the MsaRb IgG mAb, mice were i.p. injected with PBS, Fc-μTP-L309C (HEK) or one of three trivalent Fc multimers(CC, SIF1 and Q1) at 50 mg/kg. After the injection of MsaRb, mice were placed individually in metabolic cages (Tecniplast) to collect urine over a period of 24 hours. Urine albumin levels were measured with an ELISA kit (Bethyl Laboratories) and albuminuria per mouse is plotted as μg/24 hr.

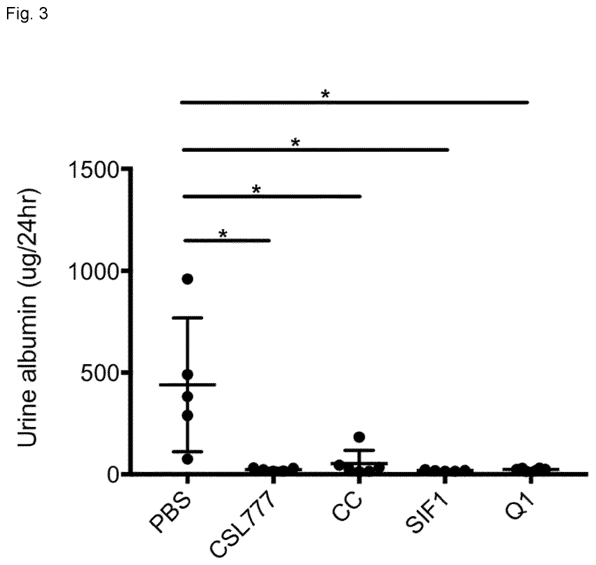

As shown in FIG. 3, urine albumin levels were significantly reduced in mice treated with Fc-μTP-L309C or the trivalent Fc multimers. (PBS vs Fc-μTP-L309C: p=0.0220; PBS vs CC: p=0.0190; PBS vs SIF1: p=0.0208; PBS vs Q1: p=0.0119). Table 3 shows the measured values.

TABLE 3

| Compound | Urine Albumin (ug/24 hr)* |
|---|---|
| PBS | 440 ± 146.9 |
| Fc-μTP-L309C | 23.38 ± 3.31 |
| CC | 52.69 ± 26.69 |
| SIF1 | 18.32 ± 1.37 |
| Q1 | 23.7 ± 2.58 |

*Mean ± SEM; student test

### Example 5: Fc-μTP-L309C Inhibits Dose-Dependently Anti-GBM Glomerulonephritis

The dose-response effect of Fc-μTP-L309C generated from HEK293 cells [Fc-μTP-L309C (HEK)] was determined in an in vivo model of anti-glomerular basement membrane (GBM) glomerulonephritis.

Briefly, anti-GBM glomerulonephritis was induced in C57BL/6 mice by intravenously (i.v., tail vein) injecting 1 mg of polyclonal rabbit anti-GBM antibody at day 0, followed by intraperitoneal (i.p.) injection on day 6 with 2 mg of the mouse monoclonal anti-rabbit IgG (MsaRb IgG produced from hybridoma CRL-1753 (ATCC)). Mice were i.p. injected with PBS or Fc-μTP-L309C at 1, 5, 10, 20 or 50 mg/kg on day 6, which was approximately one hour before the injection with the MsaRb IgG mAb. After the injection of MsaRb, mice were placed individually in metabolic cages (Tecniplast) to collect urine over a period of 24 hours. Urine albumin levels were measured with an ELISA kit (Bethyl Laboratories) and albuminuria per mouse is plotted as μg/24 hr.

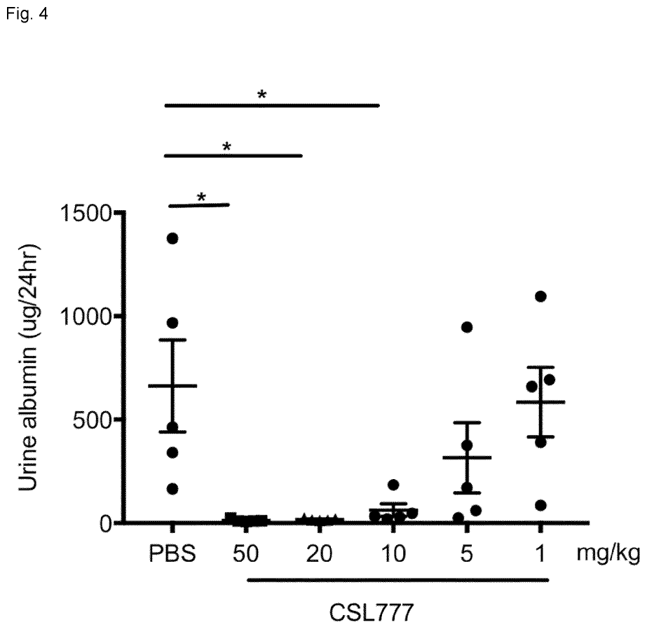

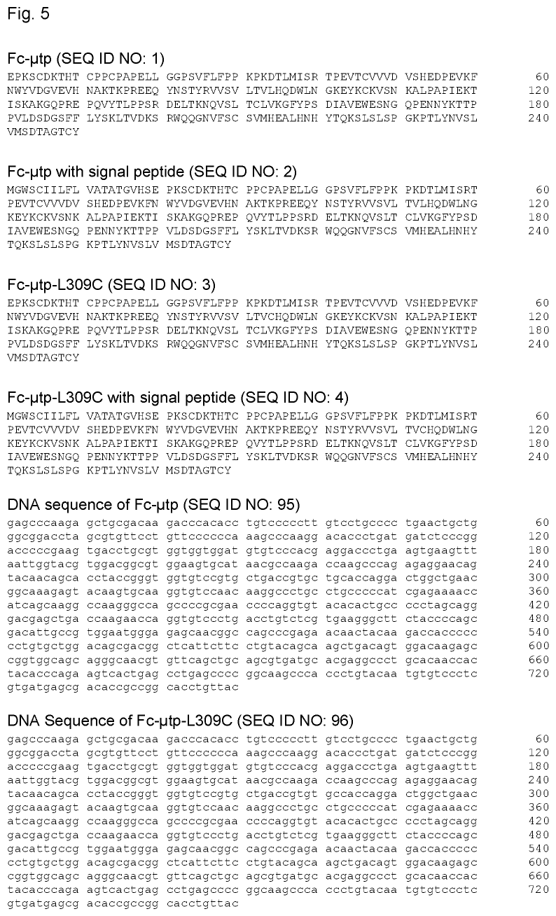

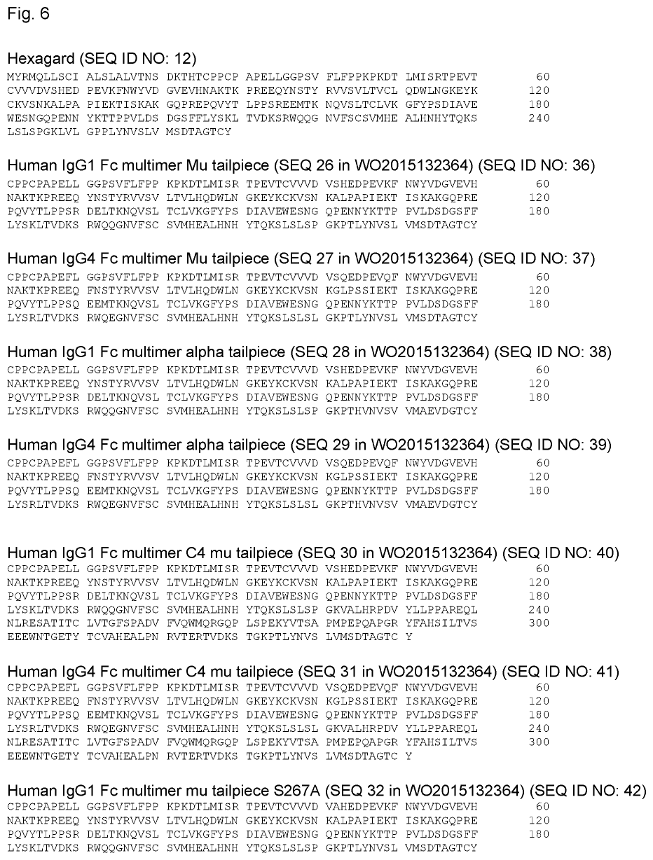

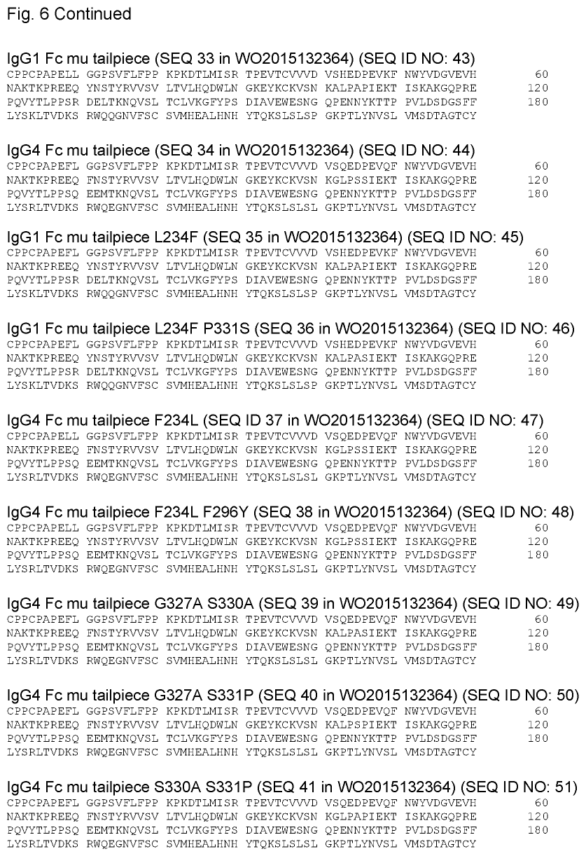

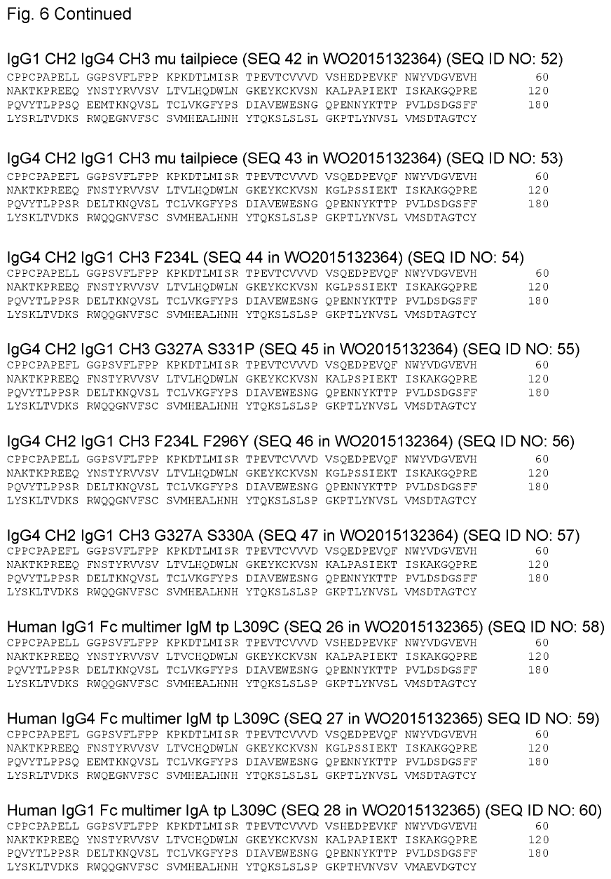

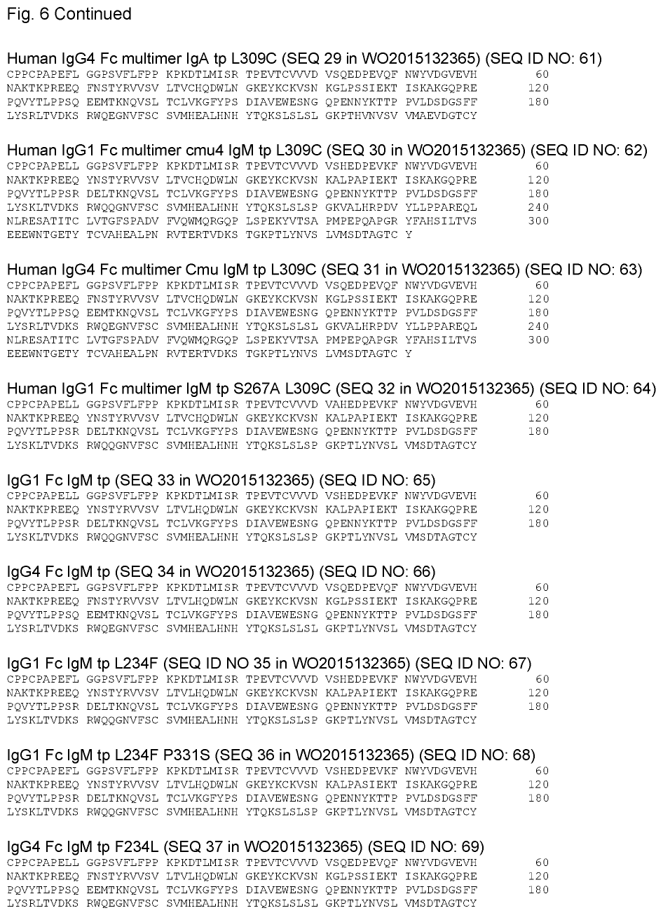

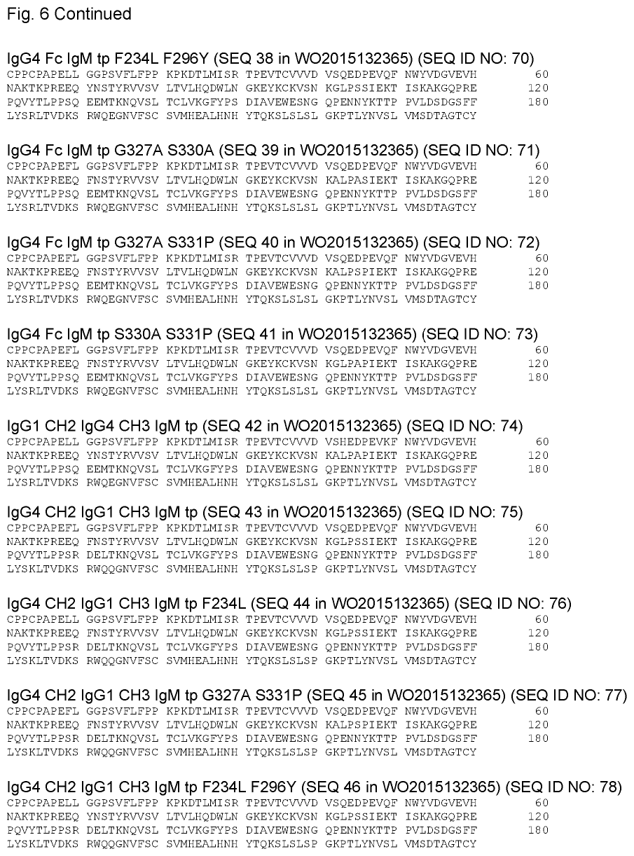

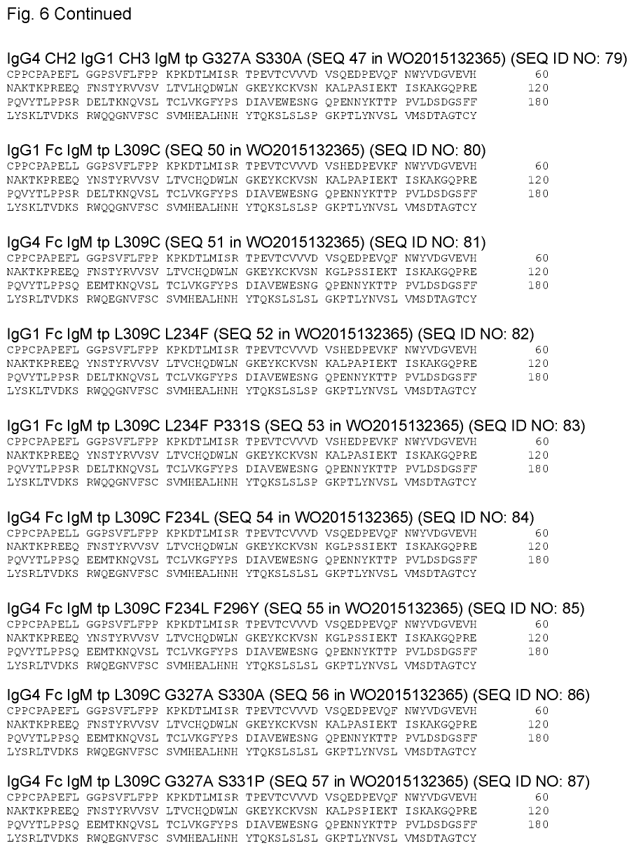

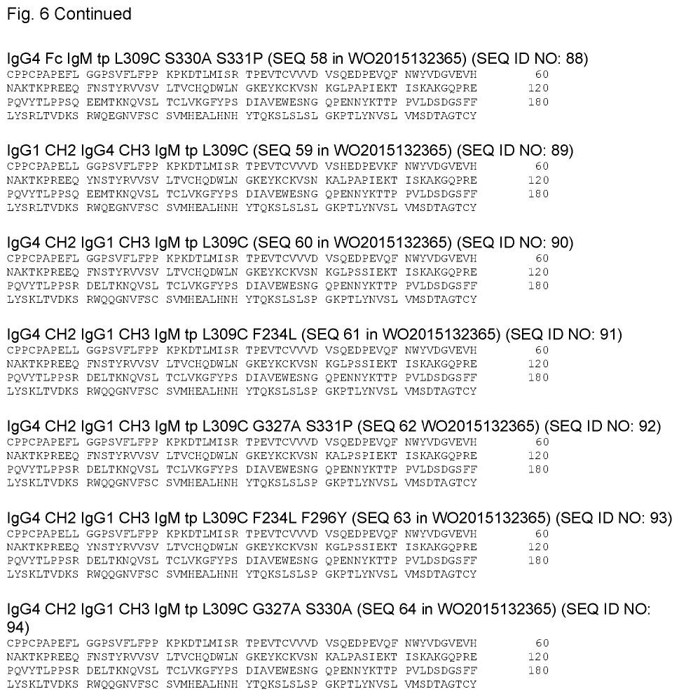

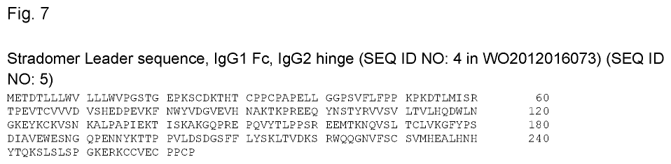

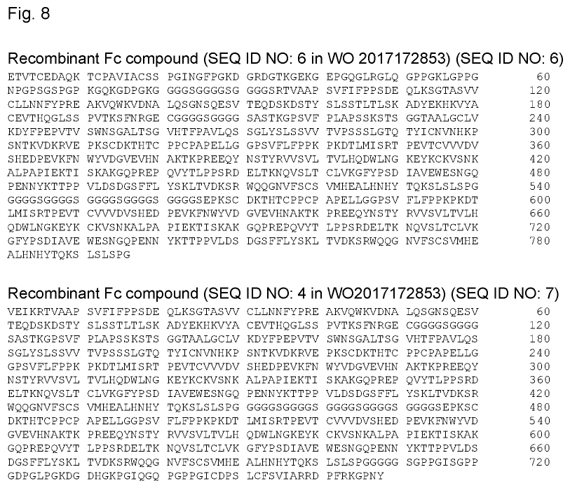

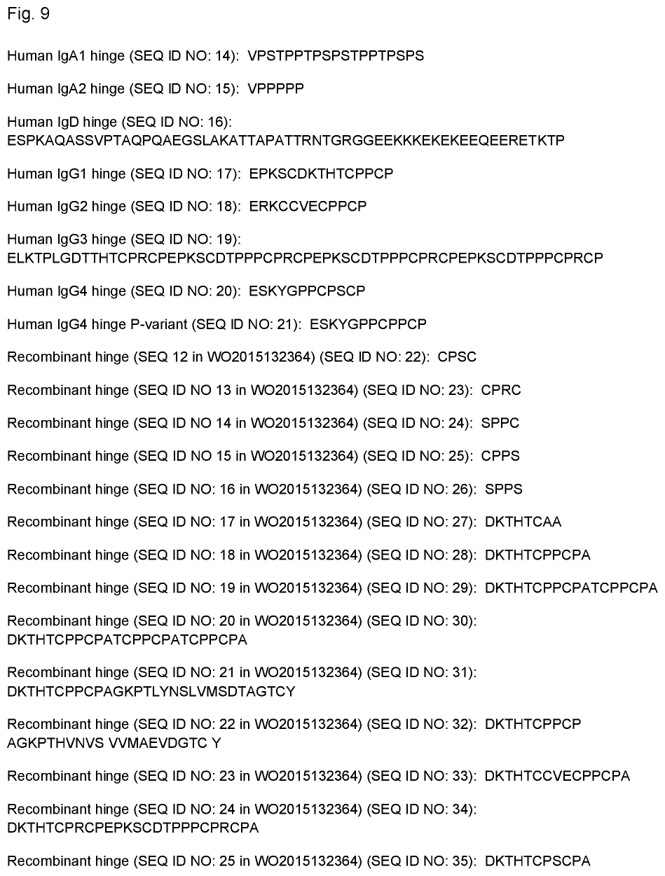

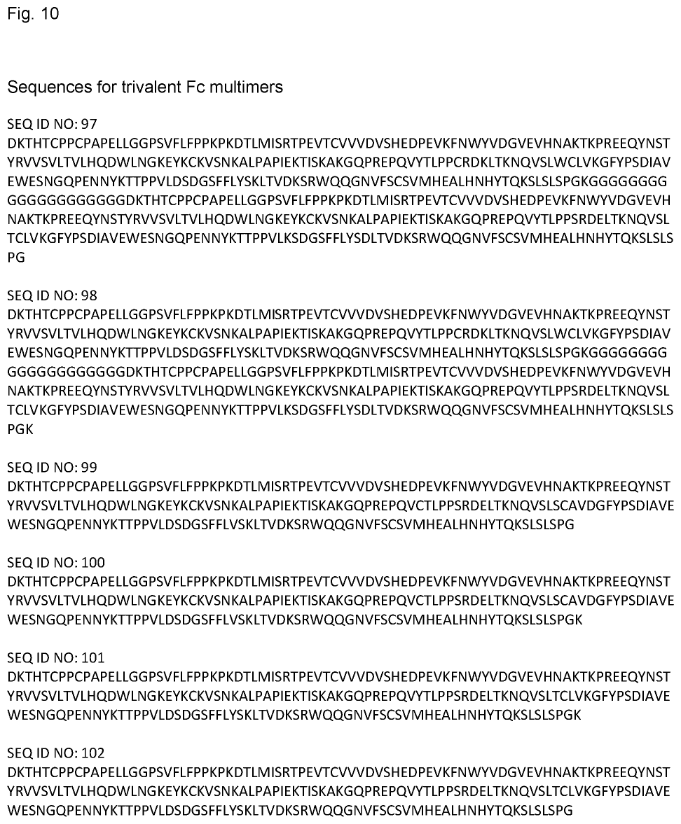

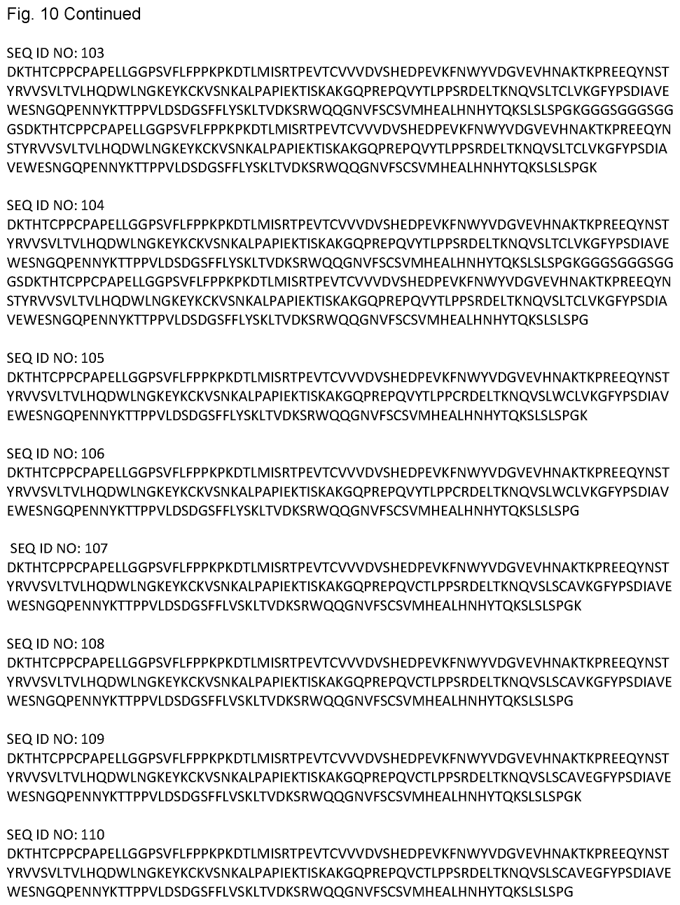

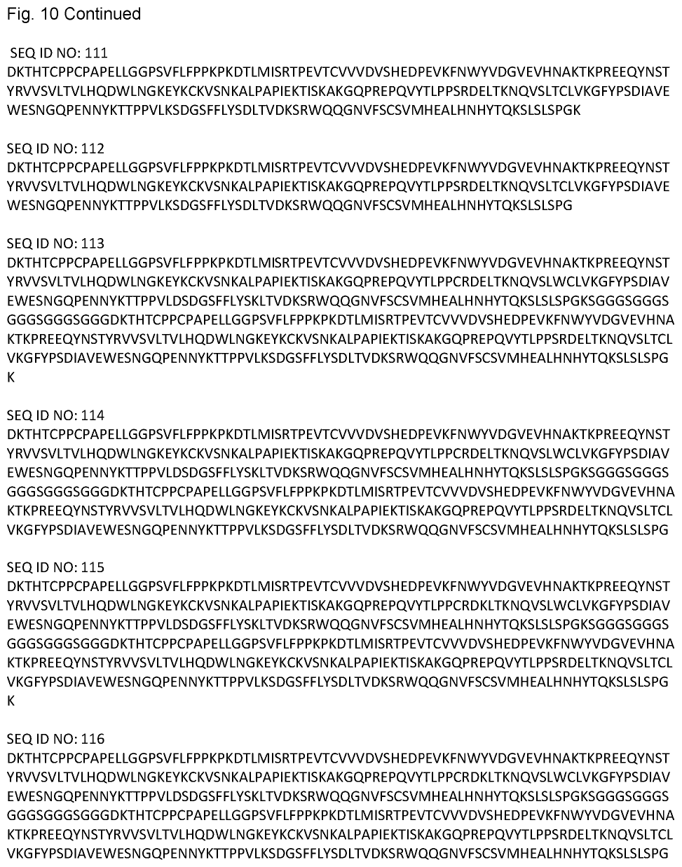

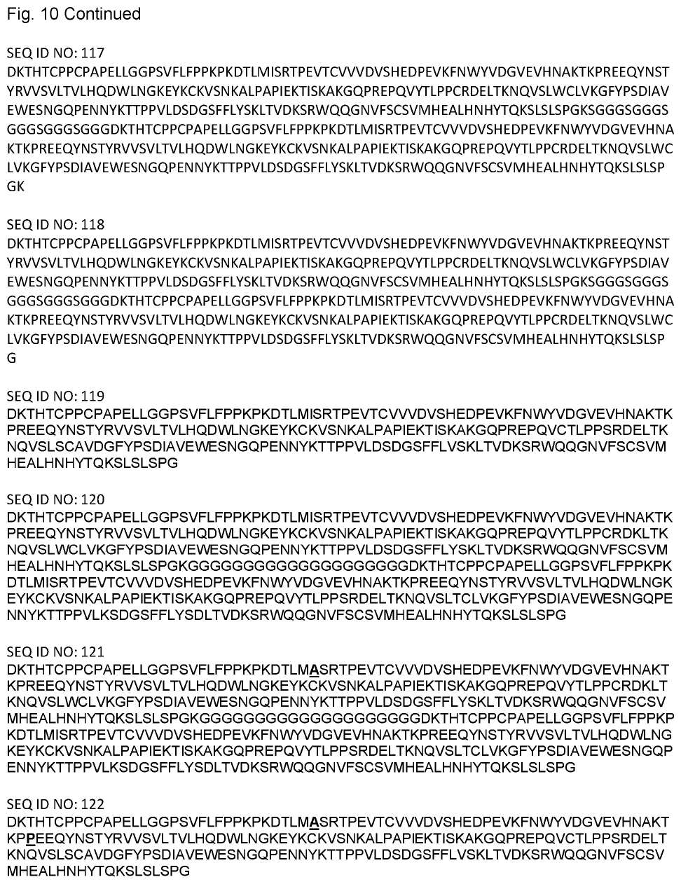

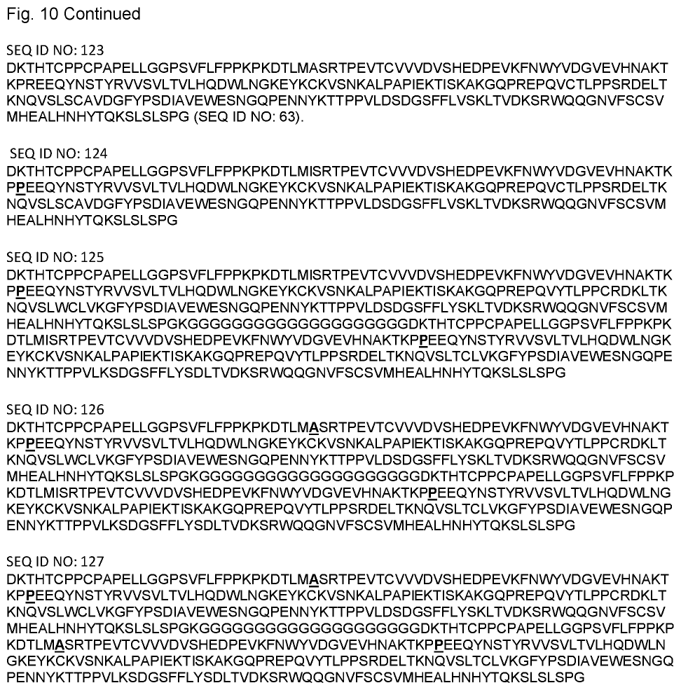

As shown in FIG. 4, Fc-μTP-L309C inhibited anti-GBM antibody-induced glomerulonephritis in a dose-dependent manner (PBS vs Fc-μTP-L309C at 50 mg/kg: p=0.0194; PBS vs Fc-μTP-L309C at 20 mg/kg: p=0.0201; PBS vs Fc-μTP-L309C at 10 mg/kg: p=0.0286; PBS vs Fc-μTP-L309C at 5 mg/kg: p=0.2505; PBS vs Fc-μTP-L309C at 1 mg/kg: p=0.7875). Table 4 shows the measured values.

TABLE 4

| Compound | Urine Albumin (ug/24 hr)* |
|---|---|
| PBS | 662 ± 222.8 |
| Fc-μTP-L309C at 50 mg/kg | 12.54 ± 2.91 |
| Fc-μTP-L309C at 20 mg/kg | 17.56 ± 2.21 |
| Fc-μTP-L309C at 10 mg/kg | 62.8 ± 30.8 |
| Fc-μTP-L309C at 5 mg/kg | 315.5 ± 169.1 |
| Fc-μTP-L309C at 1 mg/kg | 584.2 ± 168.1 |

*Mean ± SEM; student test

1.-14. (canceled)

15. A method of treating an immune complex-mediated kidney disorder in a patient in need thereof, comprising administering to the patient a therapeutically effective amount of an IgG Fc multimer, wherein the Fc multimer lacks any mutation to increase binding affinity of the Fc multimer to C1q.

16. The method of claim 15, wherein the mutation to increase binding affinity of the Fc multimer to C1q is at least one of S267E, H268F, S324T, N297A, T299A, P238D, E233P, G236R, L234V, E233P, L234A, L235A, P238D, D265A, D265W, N297A, N297Q, T299A, and L328F in an IgG1 Fc domain of the Fc multimer.

17. The method of claim 15, wherein the Fc multimer comprises two to six IgG Fc fusion monomers, wherein each Fc fusion monomer comprises two Fc fusion polypeptide chains, and wherein each Fc fusion polypeptide chain comprises an IgG Fc polypeptide and a multimerization domain.

18. The method of claim 15, wherein the Fc multimerization domain does not include an IgG2 hinge.

19. The method of claim 15, wherein the Fc multimer is not a stradomer.

20. The method of claim 15, wherein the Fc multimer comprises six human IgG1 Fc fusion monomers, wherein each Fc fusion monomer comprises two human Fc fusion polypeptide chains and each Fc fusion polypeptide chain comprises a human IgG1 Fc polypeptide and a human IgM tailpiece.

21. The method of claim 20, wherein the IgM tailpiece in each Fc fusion polypeptide chain comprises 18 amino acids fused with 232 amino acids at a C-terminus of a constant region of the IgG1 Fc polypeptide.

22. The method of claim 20, wherein each IgG1 Fc polypeptide comprises a leucine to cysteine mutation at position 309.

23. The method of claim 22, wherein the Fc fusion polypeptide chain comprises SEQ ID NO:3.

24. The method of claim 15, wherein the Fc multimer comprises four polypeptides that form three Fc monomers, wherein the first polypeptide comprises a first Fc polypeptide, a first linker, and a second Fc polypeptide, wherein the second polypeptide comprises a third Fc polypeptide, a second linker, and a fourth Fc polypeptide, wherein the third polypeptide comprises a fifth Fc polypeptide, wherein the fourth polypeptide comprises a sixth Fc polypeptide, wherein the first Fc polypeptide and the third Fc polypeptide form the first Fc monomer, wherein the fifth Fc polypeptide and the second Fc polypeptide form the second Fc monomer, and wherein the sixth Fc polypeptide and fourth Fc polypeptide form the third Fc monomer.

25. The method of claim 24, wherein the first polypeptide and the second polypeptide are identical, and further wherein the third polypeptide and the fourth polypeptide are identical.

26. The method of claim 25, wherein the first polypeptide and the second polypeptide comprise SEQ ID NO:120 and the third polypeptide and the fourth polypeptide comprise SEQ ID NO:119; or wherein the first polypeptide and the second polypeptide comprise SEQ ID NO:125 and the third polypeptide and the fourth polypeptide comprise SEQ ID NO:124; or wherein the first polypeptide and the second polypeptide comprise SEQ ID NO:126 and the third polypeptide and the fourth polypeptide comprise SEQ ID NO:122; or wherein the first polypeptide and the second polypeptide comprise SEQ ID NO:107 or SEQ ID NO:108, and the third polypeptide and the fourth polypeptide comprise SEQ ID NO:113 or SEQ ID NO:114.

27. The method of claim 15, wherein the immune complex-mediated kidney disorder is nephritis, glomerulonephritis, interstitial nephritis, anti-glomerular basement membrane (anti-GBM) disease, Goodpasture syndrome, autoimmune kidney disease, lupus nephritis, membranous nephropathy, membranoproliferative glomerulonephritis (MPGN), or Bright's disease.

28. The method of claim 15, wherein the Fc multimer inhibits complement-dependent cytotoxicity and/or antibody-dependent cellular cytotoxicity.
